(19) United States
(12) Patent Application Publication
(10) Pub. No.: US 2024/0342207 A1
(43) Pub. Date: Oct. 17, 2024

(54) MODIFIED NUCLEIC ACID COMPOSITIONS AND ASSOCIATED METHODS FOR TREATMENT OF PHENYLKETONURIA

(71) Applicant: BOARD OF REGENTS, THE UNIVERSITY OF TEXAS SYSTEM, Austin, TX (US)

(72) Inventors: Liuqing YANG, Houston, TX (US); Chunru LIN, Houston, TX (US); Sergey D. EGRANOV, Houston, TX (US); Yajuan LI, Claymont, DE (US)

(21) Appl. No.: 18/294,680

(22) PCT Filed: Aug. 2, 2022
(86) PCT No.: PCT/US2022/074414

§ 371 Date: Feb. 2, 2024

Related U.S. Application Data
(60) Provisional application No. 63/228,322, filed on Aug. 2, 2021.

(51) Int. Cl. A61K 31/7105; A61K 48/00; A61P 3/00; C12N 15/11

(52) U.S. Cl. CPC A61K 31/7105; A61K 48/0075; A61P 3/00; C12N 15/11; C12N 2310/315; C12N 2310/322; C12N 2310/3517

(57) ABSTRACT

This disclosure provides modified nucleic acids and compositions thereof that are able to mimic the human HULC lncRNA and/or the mouse Pair lncRNA and enhance phenylalanine hydroxylase enzyme (PAH)-substrate binding affinity, PAH-cofactor binding affinity, and/or PAH enzymatic conversion of phenylalanine to tyrosine. Also provided are methods of using the modified nucleic acids and compositions thereof to treat subjects having or suspected of having phenylketonuria and hyperphenylalaninemia.

### CROSS-REFERENCE TO RELATED APPLICATIONS

This application claims priority to and the benefit of U.S. Provisional Patent Application Ser. No. 63/228,322, filed Aug. 2, 2021, the contents of which is incorporated herein by this reference as if fully set forth herein.

### REFERENCE TO A SEQUENCE LISTING

The official copy of the sequence listing is submitted electronically via Patent Center as an XML formatted sequence listing with a file named 1336646_seglist.xml, created on Aug. 1, 2022, and having a size of 93 KB, and is filed concurrently with the specification. The sequence listing contained in this XML formatted document is part of the specification and is herein incorporated by reference in its entirety.

### BACKGROUND

Phenylketonuria (PKU, OMIM 261600) and its milder variant hyperphenylalaninemia (HPA) are genetic disorders caused by a deficiency in the hydrolysis of L-phenylalanine (Phe) to L-Tyrosine (Tyr) by the phenylalanine hydroxylase enzyme (PAH). Roughly 1 in every 10,000 infants is affected by this disease. Over 1,000 PAH variants (PAHvdb database; biopku.org/home/pah.asp) have been identified and associated with PAH deficiency. Most of the variants are missense, usually resulting in protein misfolding and/or impairment of catalytic functions. Based on blood Phe concentration during diagnosis or screening in the neonatal period, PKU can be categorized as PKU (Phe>900 μmol/L), mild PKU (Phe<900 but >360 μmol/L), or mild HPA (blood Phe is higher than the normal limit, but <360 μmol/L). Patients with untreated PKU exhibit brain damage, intellectual disability, behavioral issues, seizures, and psychiatric disorders. Newborns diagnosed with PKU are typically treated with a Phe-restricted diet and/or BH₄ (tetrahydrobiopterin, a PAH cofactor) supplementation. However, these compensatory lifestyle considerations can have marked, potentially negative impacts on overall quality of life. Also, patients with a severe form of PKU (Phe concentrations above 900 μmol/L and usually above 1200 μmol/L) require additional treatment considerations. These patients also frequently respond poorly to BH₄ treatment. Additional therapeutic options include enzyme supplementation therapy (e.g., using a phenylalanine ammonia lyase fusion protein such as Palynziq or a PAH fusion protein) and gene therapy (e.g., using an adenoviral vector or a related vector to correct a causative mutation, such as a PAH mutation). However, given the potential immunogenic nature of PAL/PAH enzyme supplementation and the uncertainty of gene therapy, additional therapeutic strategies are urgently needed.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, provided herein are modified nucleic acids. In some embodiments, the modified nucleic acids comprise a nucleotide sequence having at least 80% identity to any one of SEQ ID NOs: 3-7 and at least one nucleotide comprising a 2′-fluoro base modification. In some embodiments, the modified nucleic acids comprise a nucleotide sequence having at least 80% identity to any one of SEQ ID NOs: 5-7. In some embodiments, the modified nucleic acids comprise a nucleotide sequence having at least 80% identity to one of SEQ ID NOs: 3 or 4. In some embodiments, the modified nucleic acids have a length of 15 to 40 nucleotides. In some embodiments, at least one of the nucleotides of a modified nucleic acid provided herein is a ribonucleotide. In some embodiments, a majority of the nucleotides of a modified nucleic acid provided herein are ribonucleotides.

In some embodiments of the modified nucleic acids provided herein, at least 25% of the nucleotides comprise a 2′-fluoro base modification. In some embodiments, all of the internal nucleotides comprise a 2′-fluoro base modification. In some embodiments, the modified nucleic acids comprise at least one phosphorothioate bond. In some embodiments of the modified nucleic acids, the 5′ terminal nucleotide and/or the 3′ terminal nucleotide is attached via phosphorothioate bond.

In some embodiments, a modified nucleic acid provided herein comprises a tag attached to the 3′ terminal nucleotide and/or the 5′ terminal nucleotide. In some embodiments, the tag comprises an apolipoprotein E peptide. In some embodiments, the apolipoprotein E peptide comprises an amino acid sequence having at least 90% identity to SEQ ID NO:9. In some embodiments, the tag comprises N-acetylgalactosamine.

In some embodiments, the modified nucleic acids provided herein increase the affinity of a phenylalanine hydroxylase (PAH) for a PAH substrate and/or a PAH cofactor.

Also provided herein are nanoparticles comprising any of the modified nucleic acids described herein.

Also provided herein are pharmaceutical preparations. In some embodiments, the pharmaceutical preparations comprise any of the modified nucleic acids described herein and a pharmaceutically acceptable carrier. In some embodiments, the pharmaceutical preparations comprise any of the nanoparticles described herein and a pharmaceutically acceptable carrier.

Also provided herein are methods for treating a subject comprising administering to the subject a therapeutically effective amount of any of the pharmaceutical preparations described herein. In some embodiments, the subject has been diagnosed with phenylketonuria or hyperphenylalaninaemia. In some embodiments, the subject is suspected to have phenylketonuria or hyperphenylalaninaemia. In some embodiments, the subject has symptoms suggestive of phenylketonuria or hyperphenylalaninaemia. In some embodiments, the subject has a mutation in a phenylalanine hydroxylase gene. In some embodiments, the mutation is R408W.

In some embodiments of the methods provided herein, the pharmaceutical preparation is administered via intravenous injection, subcutaneous injection, and/or intraperitoneal injection. In some embodiments, administration of the pharmaceutical preparation results in increased enzymatic conversion of phenylalanine to tyrosine by a phenylalanine hydroxylase (PAH) in one or more cells of the subject. In some embodiments, administration of the pharmaceutical preparation results in increased affinity of phenylalanine hydroxylase (PAH) for a PAH substrate and/or a PAH cofactor. In some embodiments, the PAH substrate is phenylalanine. In some embodiments, the PAH cofactor is tetrahydrobiopterin (BH4).

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows Northern blot detection of the expression of Pair in Pair^{-/-} mouse models of human PKU disease, according to certain aspects of this disclosure. #-actin was used as a loading control.

FIG. 2 shows a comparison of body weight of indicated female (top panel; n=6, 12, 8) and male (bottom panel, n=6, 10, 5) Pair^{-/-} mice and controls at 3-12 weeks, according to certain aspects of this disclosure. Data shown as mean±SD, one-way ANOVA.

FIG. 3 shows blood phenylalanine concentrations in Pair^{-/-} mice and controls, according to certain aspects of this disclosure. Phenylalanine concentration for indicated mice was tested every 2 weeks starting from 4 weeks of age (n=5, 5, 7 animals). Mean±SD, one-way ANOVA.

FIG. 4 shows overall survival and seizure-free survival of Pair^{-/-} mice and controls, according to certain aspects of this disclosure. The top panel shows a cumulative survival curve of cohorts of indicated littermates (n=11, 13, 28, log-rank test). The bottom panel shows a cumulative seizure-free survival curve of cohorts of indicated littermates (n=11, 13, 28, log-rank test).

FIG. 5 shows quantification of brain weight and tyrosine hydroxylase (TH) positive neurons in Pair^{-/-} mice and controls, according to certain aspects of this disclosure. The left panel shows scatter plots representing brain weight quantification for both female and male indicated littermates at the age of 12 months (n=5, 5). For female and male, Pair^{+/+} mice are shown in the left column, Pair^{+/-} mice are shown in the middle column, and Pair^{-/-} mice are shown in the right column. Mean±SD, one-way ANOVA. The right panel shows quantification of coronal sections of adult mouse brains subjected to immunohistochemical staining and quantitative data for tyrosine hydroxylase (TH) positive neurons in the substantia nigra compact (SNc)/ventral tegmental area (VTA) of indicated mice at the age of 12 months (n=5, 5). Mean±SD, one-way ANOVA. (n.s., p>0.05; *, p<0.05; **, p<0.01; ***, p<0.001; ****, p<0.0001).

FIG. 6 shows protein candidates that interact with Pair, according to certain aspects of this disclosure. Protein candidates were revealed by LC-MS. X-axis indicates different experimental groups.

FIG. 7 shows that Pair and HULC associate with PAH, according to certain aspects of this disclosure. Shown are the results of electrophoretic mobility shift assays using recombinant His-tagged PAH and [γ-³²P]-labeled Pair nt 467-488 (top panel) or HULC nt 180-202 (bottom panel) wild-type or mutant oligonucleotides. Unlabeled Pair or HULC wild-type or mutant RNA oligonucleotides were included as competitors.

FIG. 8 shows that Pair and HULC associate with PAH, according to certain aspects of this disclosure. The left panel shows the result of an MS2-TRAP assay using Pair^{-/-} hepatocytes expressing indicated plasmids was performed by immunoblotting using indicated antibodies. The right panel shows immunoblotting (bottom) or autoradiography (top) of CLIP assay using indicated hepatocytes expressing indicated plasmids.

FIG. 9 shows that HULC and Pair modulate the enzymatic activities of PAH, according to certain aspects of this disclosure. The top panel shows streptavidin pull-down using Bio-Phe/Bio-BH₄ followed by immunoblotting detection using anti-PAH antibody in indicated hepatocytes. The bottom panels show ELISA measurement of the percentage of PAH-associated BH₄ (bottom left panel) or PAH-associated Phe (bottom right panel) in indicated hepatocytes expressing indicated mimics. For each pair of columns in the bottom left and bottom right panels, the left column shows αPAH data and the right column shows αIgG data. Mean±SEM, n=5 independent experiments, one-way ANOVA. (n.s., p>0.05; *, p<0.05; **, p<0.01; ***, p<0.001).

FIG. 10 shows that HULC mimics facilitate PAH-Phe and PAH-BH₄ interactions, according to certain aspects of this disclosure. The log 2 of relative fold change of His-PAH WT/mutants and Biotin-Phe (top panel) or His-PAH WT/mutants and Biotin-BH₄ (bottom panel) binding affinity in the presence of indicated lncRNA mimics is shown. In both panels, for each set of 3 columns, the left column shows LINK-A mimic data, the center column shows HULC mimic data, and the right column shows MULC mut mimic data. The fold change was normalized using His-PAH WT in the presence of LINK-A mimic.

FIG. 11 shows that HULC mimics facilitate PAH-Phe and PAH-BH₄ function, according to certain aspects of this disclosure. The top panel shows phenylalanine and tyrosine concentrations in neonatal blood spots in the presence of His-tagged wild-type PAH/mutants and indicated lncRNA mimics. In the HULC mimic data, PAH mutants that showed improved enzymatic activities in converting Phe to Tyr are indicated with #symbols, and PAH mutants that did not show improved enzymatic activities are indicated with * symbols. Mean±SD, n=3 independent experiments, student's t-test. LncRNA mimic representing LINK-A nt 1100-1117 was included as negative control. The bottom panel shows determination of k_{cat} of recombinant PAH WT or indicated mutant proteins in the presence of indicated mimics. For each set of 3 columns in the bottom panel, the left column shows Scr control data, the middle column shows HULC data, and the right column shows HULC mut data. Mean±SD, n=3 independent experiments, one-way ANOVA. (n.s., p>0.05; *, p<0.05; **, p<0.01; ***, p<0.001).

FIG. 12 shows that GalNAc-HULC lncRNA mimics can reduce blood Phe concentrations in Pair^{-/-} mice, according to certain aspects of this disclosure. Blood Phe concentrations were monitored every day for short-term treatment in female (n=9, 11, 10; top panel) and male (n=9, 11, 10; bottom panel) Pair^{-/-} mice with indicated mimics, student's t-test.

FIG. 13 shows that GalNAc-HULC lncRNA mimics can reduce blood Phe concentrations in Pair^{-/-} mice, according to certain aspects of this disclosure. Blood Phe concentrations were monitored every other day for medium-term treatment in female (n=5, 5; top panel) and male (n=5, 5; bottom panel) Pair^{-/-} mice treated with indicated mimics, student's t-test.

FIG. 14 shows that GalNAc-HULC lncRNA mimics can reduce blood Phe concentrations in Pah^{R408W/R408W} mice, according to certain aspects of this disclosure. Blood Phe concentrations were monitored every day for short-term treatment in female (top panel) or male (bottom panel) Pah^{R408W/R408W} mice treated with indicated mimics (n=9, 9, 8), student's t-test.

FIG. 15 shows that GalNAc-HULC lncRNA mimics can reduce blood Phe concentrations in Pah^{R408W/R408W} mice, according to certain aspects of this disclosure. Blood Phe concentrations were monitored every other day for medium-term treatment in female (top panel) or male (bottom panel) Pah^{R408W/R408W} mice treated with indicated mimics (n=5, 5), student's t-test.

FIG. 16 shows that GalNAc-HULC lncRNA mimics can reduce blood tyrosine concentrations in Pah^{R408W/R408W} mice, according to certain aspects of this disclosure. Measurement of blood tyrosine concentrations for medium-term treatment in female (n=5, 5) and male (n=5, 5) Pah^{R408W/R408W} mice treated with indicated mimics, student's t-test. For female and male, the left column shows GalNAc-Scr data, and the right column shows GalNAc-HULC data.

FIG. 17 shows that GalNAc-HULC lncRNA mimics can improve phenylalanine clearance in Pah^{R408W/R408W} mice, according to certain aspects of this disclosure. Shown are the results of phenylalanine clearance tests of female (top panel) or male (bottom panel) Pah^{R408W/R408W} mice subjected to a Phe-free diet and pretreatment with indicated mimics (n=5, 5). Mean±SD, student's t-test.

FIG. 18 shows that GalNAc-HULC lncRNA mimics can improve phenylalanine clearance in Pah^{R408W/R408W} mice, according to certain aspects of this disclosure. Shown are the area under the curve (AUC) results from phenylalanine clearance tests in female or male Pah^{R408W/R408W} mice subjected to a Phe-free diet and pretreatment with indicated mimics (n=5, 5). Mean±SD, student's t-test. For female and male, the left column shows GalNAc-Scr data, and the right column shows GalNAc-HULC data.

FIG. 19 shows that GalNAc-HULC lncRNA mimics can improve phenylalanine tolerance in Pah^{R408W/R408W} mice, according to certain aspects of this disclosure. Shown are the results of phenylalanine tolerance tests of female (top panel) or male (bottom panel) Pah^{R408W/R408W} mice subjected to a Phe-free diet and pretreatment with indicated mimics for three days, followed by water containing 0, 0.75, 1.5, 3.0, or 6.0 mg/ml of Phe (with the dose increasing every two days), n=5, 5 animals per group, student's t-test.

FIG. 20 shows that GalNAc-HULC lncRNA mimics can have an additive effect with sapropterin in reducing blood phenylalanine concentrations, according to certain aspects of this disclosure. Blood Phe concentrations were monitored every day for short-term treatment in Pah^{R408W/R408W} mice subjected to sapropterin alone or in combination with indicated mimics (n=5 animals per group). Mean±SD, one-way ANOVA. (n.s., p>0.05; *, p<0.05; **, p<0.01; ***, p<0.001; ****, p<0.0001).

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Terminology

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e. at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

As used herein, a “nucleotide analog” has one or more modifications, such as chemical moieties, which replace, remove and/or modify any of the components (e.g., nitrogenous base, five-carbon sugar, or phosphate group(s)) of a native nucleotide. Nucleotide analogs may be either incorporable or non-incorporable by a polymerase in a nucleic acid polymerization reaction. The base of a nucleotide may be any of adenine, cytosine, guanine, thymine, or uracil, or analogs thereof. Optionally, a nucleotide has an inosine, xanthine, hypoxanthine, isocytosine, isoguanine, nitropyrrole (including 3-nitropyrrole) or nitroindole (including 5-nitroindole) base. Nucleotides may include, but are not limited to, ATP, UTP, CTP, GTP, ADP, UDP, CDP, GDP, AMP, UMP, CMP, GMP, dATP, dTTP, dUTP, dCTP, dGTP, dADP, dTDP, dCDP, dGDP, dAMP, dTMP, dCMP, and dGMP.

Unless otherwise indicated, a particular nucleic acid sequence also implicitly encompasses conservatively modified variants thereof, alleles, orthologs, SNPs, and complementary sequences as well as the sequence explicitly indicated.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et la. “Adding amino acids to the genetic repertoire,” 9(6): 548-54 (2005)); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

### II. Introduction

Provided herein are modified nucleic acids and associated compositions and methods for treatment of patients with Phenylketonuria (PKU) and hyperphenylalaninemia (HPA). In some embodiments, the modified nucleic acids are mimics of the Homo sapiens hepatocellular carcinoma up-regulated long non-coding RNA (HULC). In some embodiments, the modified nucleic acids are mimics of the Mus musculus PAH-activating lncRNA (Pair). HULC and Pair associate with PAH protein at the N-terminal regulatory domain. In some embodiments, the presence of HULC rescues PAH enzymatic activity in Pair-deficient cells and vice versa. In some embodiments, these lncRNA mimics are modified (e.g., using peptide or sugar tags) to promote targeted enrichment in the liver. In some embodiments, the lncRNA mimics enhance PAH-substrate and/or PAH-cofactor binding affinities and enhance enzymatic activity in vitro and in vivo. In some embodiments, the lncRNA mimics enhance binding affinities of mutant PAH (e.g., R408W mutation) for PAH-substrate and/or PAH-cofactor and enhance enzymatic activity of mutant PAH in vitro and in vivo. The lncRNA mimics provided herein target lncRNAs as a therapeutic strategy against a human inherited metabolic disorder. In some embodiments, combining HULC mimics and/or Pair mimics with current dietary restrictions, sapropterin supplements, and enzyme replacement or substitution therapies can further improve patient outcomes, particularly for patients with severe PKU.

PKU has long been considered a single-gene disease with an autosomal recessive inheritance pattern, in which mutations at the phenylalanine hydroxylase enzyme (PAH) locus lead to impaired enzymatic function and contribute to a hyperphenylalaninemia metabolic phenotype, subsequently resulting in cognitive phenotypes such as mental retardation. However, recent genotype-phenotype correlation analyses of PAH mutations suggested a substantial discrepancy between genotypes and their predicted metabolic or cognitive phenotypes (1, 2, 27, 28). In some patients diagnosed with PKU, no mutations could be identified in the PAH gene, suggesting that unknown factors may contribute to PKU (3). Consistent with this notion, the recent discovery of biallelic mutations in the DNAJC12 gene in patients exhibiting high blood Phe concentrations without mutations in PAH or genes involved in BH₄ metabolism (4, 30) suggested the possibility that non-PAH genes may affect PAH function and subsequently increase blood Phe concentrations.

Variations in genomic, epigenomic, transcriptomic, proteomic, and metabolic systems could all contribute to the PAH deficiency of patients with PKU (29). It has been shown that patients, even siblings sharing identical mutant PAH genotypes, can have greatly differing cognitive and metabolic phenotypes (3). This highly suggests that a PAH genotype cannot consistently and reliably predict the monogenic phenotype (3). Hence, factors modulating PAH expression, PAH protein stability, BH₄ biogenesis, and PAH enzymatic activity may play important roles in maintaining PAH enzymatic proficiency. More than 95% of genetic mutations occur at the non-coding regions of the human genome, yet the functional importance of long intergenic noncoding RNAs (lincRNAs) and long noncoding RNAs (lncRNAs), RNA transcripts longer than 200 nucleotides with low coding potential, in human genetic diseases remains elusive.

Provided in the Examples and disclosures herein is a demonstration that depletion of the mouse lincRNA 2210408F21Rik (NCBI accession number NR_040259) (referred to herein as Pair, or PAH-activating lincRNA) leads to phenotypes that model human PKU, including hypopigmentation, growth retardation, seizures, and neuronal loss. Pair is a 734 nucleotide lincRNA expressed from a gene on mouse chromosome 6. As described in Example 1 herein, Pair is highly upregulated in the livers of adult mice compared to those of embryos.

Further provided herein are data demonstrating that the human lncRNA HULC (NCBI accession number NR_004855.2) appears to behave similarly to Pair. HULC, or Homo sapiens hepatocellular carcinoma up-regulated long non-coding RNA, is a 500-nucleotide noncoding RNA expressed from a gene on human chromosome 6. HULC has previously been shown to be upregulated in hepatocellular carcinoma and other cancer types, and has been suggested to promote tumorigenesis through modulating cancer signaling pathways and the status of microRNAs. As described in Example 2 herein, HULC associates with human PAH enzyme and is specifically expressed in normal liver tissue.

Long noncoding RNA mimics as a therapeutic option has at least the following advantages over other therapeutic strategies. 1) Synthesis of lncRNA mimics provides flexibility to the target sequence. For example, the target sequence could be designed to improve the enzymatic activity of key enzyme(s) in other metabolic disorders. 2) lncRNA mimics can display profound stability in vivo based on nucleic acid modifications. For example, modified nucleotides and/or modified covalent bonds between nucleotides can provide nuclease resistance, as described herein. 3) lncRNA mimics can be designed with organ-targeting tags (e.g., peptides or other chemical moieties) for tissue-specific distribution. For example, as described herein, lncRNA mimics can be targeted for liver enrichment using, for example, a GalNAc tag or an apolipoprotein E tag. 4) lncRNA mimics show low potential for organ toxicity, as supported by the observations herein that administration of lncRNA mimics resulted in no detectable effects on liver or kidney function.

### III. Modified Nucleic Acids

In one aspect, provided herein are modified nucleic acids. In some embodiments, the modified nucleic acids interact with the enzyme phenylalanine hydroxylase in vitro and/or in vivo to promote the enzymatic conversion of phenylalanine to tyrosine. In some embodiments, the modified nucleic acids are useful for treatment of patients with PKU or HPA. In some embodiments, a modified nucleic acid comprises a HULC mimic (e.g., as described above), a Pair mimic (e.g., as described above), or a HULC mimic and a Pair mimic.

In some embodiments, the modified nucleic acids provided herein comprise a nucleotide sequence having at least 60% identity (e.g., at least about 65%, at least 70%, at least 75%, at least 80%, at least 85%, at least 90%, at least 92%, at least 94%, at least 95%, at least 96%, at least 97%, at least 98%, at least 99%, or 100% identity) to a portion of wild-type HULC RNA (SEQ ID NO: 1) and/or wild-type Pair RNA (SEQ ID NO:2). In some embodiments, the modified nucleic acids comprise a nucleotide sequence having at least 60% identity to nucleotides 460-496 of wild-type Pair RNA (SEQ ID NO:3). In some embodiments, the modified nucleic acids comprise a nucleotide sequence having at least 60% identity to nucleotides 470-488 of wild-type Pair RNA (SEQ ID NO:4). In some embodiments, the modified nucleic acids comprise a nucleotide sequence having at least 60% identity to nucleotides 183-216 of wild-type HULC RNA (SEQ ID NO:5). In some embodiments, the modified nucleic acids comprise a nucleotide sequence having at least 60% identity to nucleotides 183-200 of wild-type HULC RNA (SEQ ID NO:6). In some embodiments, the modified nucleic acids comprise a nucleotide sequence having at least 60% identity to nucleotides 181-201 of wild-type HULC RNA (SEQ ID NO:7). In some embodiments, the modified nucleic acids comprise a nucleotide sequence having at least 80% identity to SEQ ID NO:7.

In some embodiments, the modified nucleic acids have a length of 10 to 50 nucleotides (e.g., 10 to 50 nucleotides, 10 to 45 nucleotides, 15 to 40 nucleotides, 15 to 35 nucleotides, 15 to 30 nucleotides, 10 to 25 nucleotides, 15 to 25 nucleotides, 10 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 40 nucleotides, 45 nucleotides, or 50 nucleotides). In some embodiments, the modified nucleic acids have a length of 15 to 40 nucleotides. In some embodiments, the modified nucleic acids have a length of 15 to 30 nucleotides.

The modified nucleic acids provided herein may comprise ribonucleotides, deoxyribonucleotides, or any combination thereof. In some embodiments, at least one of the nucleotides of the modified nucleic acids provided herein is a ribonucleotide. In some embodiments, at least a quarter of the nucleotides of the modified nucleic acids provided herein is a ribonucleotide. In some embodiments, at least half of the nucleotides of the modified nucleic acids provided herein is a ribonucleotide. In some embodiments, at least three-quarters of the nucleotides of the modified nucleic acids provided herein is a ribonucleotide. In some embodiments, a majority of the nucleotides of the modified nucleic acid are ribonucleotides (e.g., at least 51%, at least 52%, at least 53%, at least 54%, at least 55%, at least 60%, at least 65%, at least 70%, at least 75%, at least 80%, at least 85%, at least 90%, at least 95%, at least 99%, or 100%). In some embodiments, all of the nucleotides of the modified nucleic acid are ribonucleotides. In some embodiments, at least 5% (e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%) of the nucleotides of the modified nucleic acid are ribonucleotides.

The modified nucleic acids provided herein comprise at least one nucleic acid modification. In some embodiments, the modification is selected to increase stability of the modified nucleic acid (e.g., by providing resistance to nuclease activity). In some embodiments, the modified nucleic acids comprise a modification to the sugar moiety of one or more nucleotides. For example, the modified nucleic acids can comprise a fluorine molecule at the 2′ ribose sugar position (2′-fluoro sugar modification) of one or more nucleotides. In some embodiments, the modified nucleic acids comprise a modification to one or more phosphate linkages between nucleotides. For example, the modified nucleic acids can comprise one or more phosphorothioate bonds between nucleotides. In a phosphorothioate bond, a sulfur atom is substituted for a non-bridging oxygen in the phosphate bond. In some embodiments, the modified nucleic acids comprise a modification to the nitrogenous base moiety of one or more nucleotides.

In some embodiments, the modified nucleic acids provided herein comprise any of the nucleotide modifications listed in Table 1 below. In some embodiments, the modified nucleic acids comprise any of the modifications (e.g., chemical modifications, bioconjugation, backbone modifications, nanocarriers, etc.) described in Roberts et al., 2020, Nat. Rev. Drug Discov. 19:673-694.

TABLE 1
Additional exemplary nucleic acid modifications.
Internal Modifications

| | |
|---|---|
| 2,6-Diaminopurine | DAP |
| 2′-Amino-uridine | 2′-N-U |
| 2′-Deoxy-uridine | dU |
| 2′-Fluoro-adenosine | 2′-F-A |
| 2′-Fluoro-cytidine | 2′-F-C |
| 2′-Fluoro-acetyl-cytidine | 2′-F-Ac-C |
| 2′-Fluoro-guanosine | 2′-F-G |
| 2′-Fluoro-uridine | 2′-F-U |
| 2-Aminopurine | 2AP |
| 4-Thio-uridine | 4-S-U |
| 5-Bromo-Uridine | U[5Br] |
| 5-Fluoro-uridine | U[5F] |
| 5-Iodo-uridine | U[5I] |
| 5-Methyl-cytidine | 5-M-C |
| 2′-Amino-cytidine | 2′-N-C |
| Inosine | I |
| N3-Methyl-uridine | 3-M-U |
| Purine ribonucleoside | Pu |
| Ribo-thymidine | rT |
| 5-Amino-allyl-uridine | N/A |
| Deoxy-abasic | N/A |
| N6,N6-Dimethyl-adenosine | N/A |
| C3 Spacer | SpC3 |
| C9 Spacer | SpC9 |
| C18 Spacer | SpC18 |

In some embodiments, the modified nucleic acids provided herein comprise at least one nucleotide comprising a 2′-fluoro sugar modification. In some embodiments, at least 25% of the nucleotides comprise a 2′-fluoro sugar modification. In some embodiments, at least 50% of the nucleotides comprise a 2′-fluoro sugar modification. In some embodiments, at least 75% of the nucleotides comprise a 2′-fluoro sugar modification. In some embodiments, all of the internal nucleotides comprise a 2′-fluoro sugar modification. As used herein, an “internal nucleotide” is any nucleotide in a nucleic acid except for the 3′ terminal nucleotide and the 5′ terminal nucleotide.

In some embodiments, the modified nucleic acids provided herein comprise at least one phosphorothioate bond. In some embodiments, the 5′ terminal nucleotide and/or the 3′ terminal nucleotide are attached to the nucleic acid via phosphorothioate bond.

In some embodiments, the modified nucleic acids provided herein comprise a tag (e.g., a peptide tag or a sugar tag) attached to one or more nucleotides. In some embodiments, the tag is attached to the 3′ terminal nucleotide. In some embodiments, the tag is attached to the 5′ terminal nucleotide. In some embodiments, a first tag is attached to the 3′ terminal nucleotide and a second tag is attached to the 5′ terminal nucleotide.

A variety of tags may be used in the modified nucleic acids described herein. In some embodiments, the tag comprises any of the moieties used in the bioconjugates described in Roberts et al., 2020, Nat. Rev. Drug Discov. 19:673-694. For example, in some embodiments, the tag is a lipid (e.g., cholesterol), a peptide, an aptamers, an antibody, or a sugar. In some embodiments, the tag is a localization moiety. In some embodiments, the tag is chosen to promote efficient delivery of the modified nucleic acids to a particular tissue and/or organ system in a subject. For example, modified nucleic acid tags may promote interactions between the modified nucleic acid and a cell surface receptor protein that corresponds to the tag moiety. Such interactions can result in internalization of the modified nucleic acid via receptor-mediated endocytosis. Tags with this property can be assessed by measuring the concentration of a tagged modified nucleic acid (e.g., using immunohistochemistry, dot blot, western blot, northern blot, isotope labeling, fluorescent labeling, etc.) in the targeted tissue type or organ relative to one or more untargeted tissue types or organs, e.g., as described in Examples 6 and 7 herein and shown in FIGS. S19E-S19H in Li et al. 2021). In some instances, the modified nucleic acids comprise a tag that results in localization (enrichment) of the modified nucleic acids in the liver of a subject after administration of the modified nucleic acids to the subject. In some instances, liver enrichment is advantageous because PAH-mediated phenylalanine metabolism occurs primarily in the liver. In some instances, liver enrichment is advantageous because PAH is primarily expressed in the liver. In some instances, liver enrichment is advantageous because it limits undesirable side effects caused by accumulation of the modified nucleic acids in non-liver tissues.

In some embodiments, the tag comprises an apolipoprotein E (ApoE) peptide. ApoE is a mammalian fat-binding protein that plays a major role in the clearance and hepatocellular uptake of physicological lipoproteins. ApoE has been shown to promote targeting of small nucleic acids to the liver. See, e.g., Akinc, et al., 2010, Mol. Ther. 18(7):1357-1364. Without being bound by any particular theory, an ApoE peptide tag may promote localization of the modified nucleic acid to the liver via interaction with the low-density lipoprotein receptor, which is particularly abundant in liver tissue. In some embodiments, the ApoE peptide comprises an amino acid sequence having at least 80% identity (e.g., at least 85% identity, at least 90% identity, at least 92% identity, at least 94% identity, at least 95% identity, at least 96% identity, at least 97% identity, at least 98% identity, or at least 99% identity) to the amino acid sequence LRKLRKRLLLRKLRKRLL (SEQ ID NO:9).

In some embodiments, the tag comprises N-acetylgalactosamine (GalNAc). GalNAc is an amino sugar derivative of galactose and has been shown to promote targeting of small nucleic acids to the liver. See, e.g., Akinc, et al., 2010, Mol. Ther. 18(7):1357-1364 and Roberts et al., 2020, Nat. Rev. Drug Discov. 19:673-694. Without being bound by any particular theory, a GalNAc tag may promote localization of the modified nucleic acid to the liver via interaction with the highly liver-expressed asialoglycoprotein receptor 1 (ASGR1).

In some embodiments, the tag comprises more than one GalNAc moiety. In some embodiments, the tag comprises three GalNAc moieties in a triantennary arrangement.

In some embodiments, the tags described herein are attached to a nucleotide or nucleoside of the modified nucleic acid via a linker. The linker may be selected and/or optimized to produce desired effects in the modified nucleic acid. Linkers may be flexible or rigid. Flexible linkers provide a certain degree of movement or interaction between the modified nucleic acid and the tag. A rigid linker can be used to keep a fixed distance between the modified nucleic acid and the tag. Linkers may be attached to the nucleobase of a nucleotide or nucleoside of the modified nucleic acid. For example, a linker can be attached to the 5-position in a pyrimidine nucleobase or to the 7-position in a purine or deazapurine nucleobase. Linkers may also be attached to a phosphate group located at the 5′-position of of a nucleotide or nucleoside of the modified nucleic acid.

In some embodiments, the linker comprises a succinimidyl 4-(N-maleimidomethyl) cyclohexane-1-carboxylate (SMCC) derivative. In some embodiments, the linker comprises the structure shown between the peptide and the nucleic acid (“oligo”) in the schematic below:

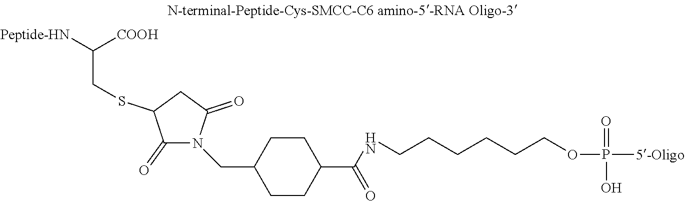

In some embodiments, the tag comprises GalNAc, and the linker comprises the structure shown between the nucleic acid (“oligonucleotide”) and the GalNAc moieties in the schematic below. The depicted structure shows attachment of three GalNAc moieties (i.e., in a triantennary arrangement). The modified nucleic acids may comprise one, two, three, or more GalNAc moieties.

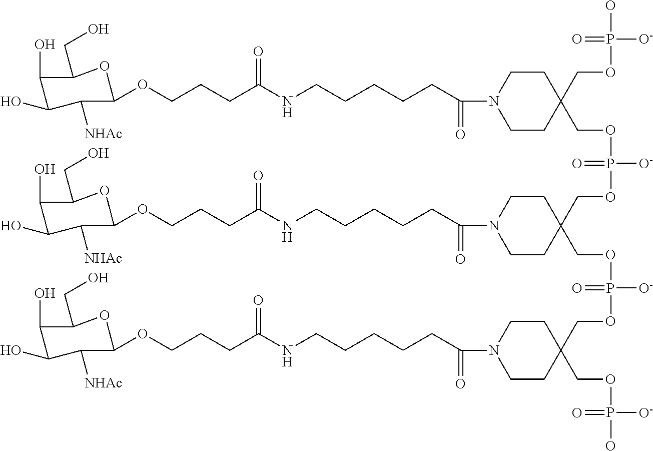 [SMILES: CC(=O)NC1C(OCCCC(=O)NCCCCCC(=O)N2CCC(COP(=O)([O-])O)(COP(=O)([O-])OCC3(COP(=O)([O-])OCC4(COP(=O)([O-])O)CCN(C(=O)CCCCCNC(=O)CCCOC5OC(CO)C(O)C(O)C5NC(C)=O)CC4)CCN(C(=O)CCCCCNC(=O)CCCOC4OC(CO)C(O)C(O)C4NC(C)=O)CC3)CC2)OC(CO)C(O)C1O]

In some embodiments, the tag comprises GalNAc, and the linker comprises the structure shown between the nucleic acid (“oligo”) and the GalNAc moieties in the schematic below:

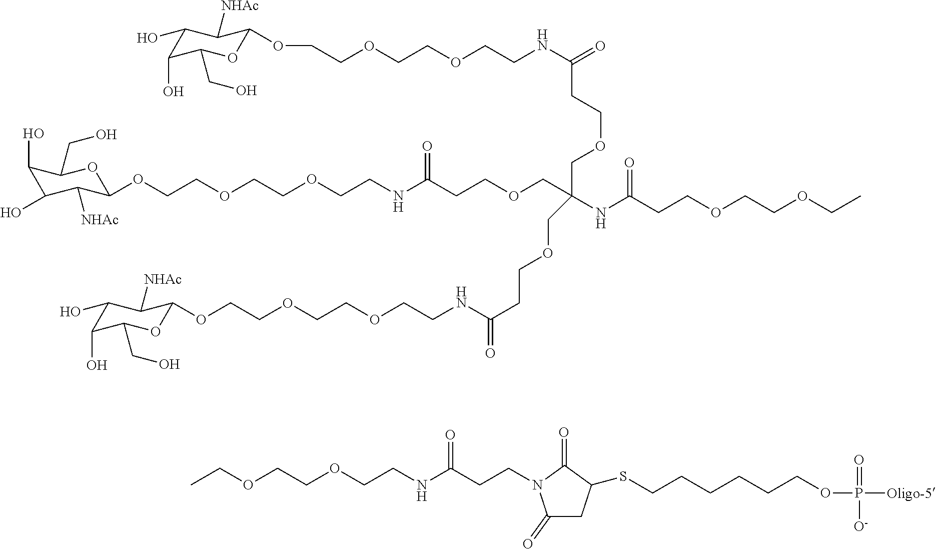

In some embodiments, the tags are attached (e.g., to the modified nucleic acid with or without a linker) via disulfide bond, amide bond, and/or click chemistry. Exemplary reactive chemical moieties include those useable in “click” chemistry, which is a class of biocompatible small molecule reactions commonly used in bioconjugation, allowing the joining of substrates of choice with specific biomolecules. Click chemistry is not a single specific reaction, but refers to a way of generating products that follow examples in nature, which also generates substances by joining small modular units. In one example, the antigenic protein may have a first reactive chemical moiety such as a clickable handle like an azide, and the binding partner(s) could have a complementary reactive handle such as, for example a strained cyclooctyne, or vice versa. When these reactive chemical moieties come into proximity when the antigenic protein and the one or binding partners interact to form a protein complex, they can react with each other to form a covalently bond between the proteins.

In some embodiments, the modified nucleic acids provided herein increase the affinity of a phenylalanine hydroxylase (PAH) for a PAH substrate and/or a PAH cofactor. In some embodiments, the PAH substrate is phenylalanine. In some embodiments, the PAH cofactor is BH₄. In some embodiments, e.g., as demonstrated in Examples 3-5 herein, the modified nucleic acid is applied to cells in vitro, and the affinity is increased relative to application of a control nucleic acid. In some embodiments, the control nucleic acid comprises a) a nucleotide sequence having less than 50% identity to SEQ ID NO:7; and b) at least one nucleotide comprising a 2′-fluoro sugar modification. In some embodiments, the PAH comprises a mutation. In some embodiments, the mutation is R408W.

In some embodiments, the modified nucleic acids provided herein increase or stimulate the enzymatic conversion of phenylalanine to tyrosine by a phenylalanine hydroxylase (PAH). In some embodiments, e.g., as demonstrated in Example 6 herein, when the modified nucleic acid is contacted to a cell (e.g., administered to a subject), the enzymatic conversion is increased relative to administration of a vehicle control. In some embodiments, the PAH comprises a mutation. In some embodiments, the mutation is R408W.

Also provided herein are host cells comprising any of the modified nucleic acids described herein. For example, a host cell can have the modified nucleic acids introduced (e.g., through electroporation, nanoparticle uptake, etc.). In some embodiments, the host cell is a mammalian cell. Also provided are nanoparticles comprising a modified nucleic acid described herein. Examples of nanoparticles that can be used in the methods and compositions described herein include, but are not limited to, gold nanoparticles, silica nanoparticles, polyethyleneglycol/polyethyleneimine particles, or lipid nanoparticles. See, for example, Lee et al. “MicroRNA delivery through nanoparticles,” Journal of Controlled Release, 313(10): 80-95 (2019).

### IV. Pharmaceutical Compositions and Formulations

The modified nucleic acids described herein are suitable for administration in vitro or in vivo. Pharmaceutical preparations comprising a modified nucleic acid of the present disclosure and a pharmaceutically acceptable carrier (excipient) are provided. In some embodiments, the pharmaceutical preparation comprises a HULC mimic (e.g., as described above), a Pair mimic (e.g., as described above), or a HULC mimic and a Pair mimic. In some embodiments, the pharmaceutical preparation comprises a nanoparticle comprising a modified nucleic acid described herein and a pharmaceutically acceptable carrier. In some embodiments, the pharmaceutical preparations comprise a modified nucleic acid having at least 60% identity (e.g., at least about 65%, at least 70%, at least 75%, at least 80%, at least 85%, at least 90%, at least 92%, at least 94%, at least 95%, at least 96%, at least 97%, at least 98%, at least 99%, or 100% identity) to a portion of wild-type HULC RNA (SEQ ID NO:1) (referred to as a “HULC mimic”). In some embodiments, the pharmaceutical preparations comprise a modified nucleic acid having at least 60% identity (e.g., at least about 65%, at least 70%, at least 75%, at least 80%, at least 85%, at least 90%, at least 92%, at least 94%, at least 95%, at least 96%, at least 97%, at least 98%, at least 99%, or 100% identity) to a portion of wild-type Pair RNA (SEQ ID NO:2) (referred to as a “Pair mimic”).

A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient (i.e., the modified nucleic acid) and to minimize any adverse side effects in the subject. The pharmaceutical compositions may further comprise a diluent, solubilizer, emulsifier, preservative, and/or adjuvant to be used with the methods disclosed herein. Such pharmaceutical compositions can be used in a subject that would benefit from administration of any of the modified nucleic acids described herein, for example, a subject with PKU or HPA.

Suitable carriers and their formulations are described in Remington: The Science and Practice of Pharmacy, 21^{st} Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous, intravenous, and/or intraperitoneal administration. In certain embodiments, the formulation comprises an appropriate amount of a pharmaceutically-acceptable salt to render the formulation isotonic. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22^{nd} Edition, Lloyd V. Allen, Jr., ed., The Pharmaceutical Press (2014). In certain embodiments, such compositions may influence the physical state, stability, rate of in vivo release and/or rate of in vivo clearance of the modified nucleic acids described herein.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such as phosphate-buffered saline or acetate-buffered saline. In certain embodiments, a composition comprising a modified nucleic acid disclosed herein can be prepared for storage by mixing the selected composition having the desired degree of purity with optional formulation agents (see Remington: The Science and Practice of Pharmacy, 22^{nd} Edition, Lloyd V. Allen, Jr., ed., The Pharmaceutical Press (2014)) in the form of a lyophilized cake or an aqueous solution. Further, in certain embodiments, a composition comprising a modified nucleic acid disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions or suspensions, including saline and buffered media) comprising a modified nucleic acid in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product that can then be delivered via a depot injection. In certain embodiments, hyaluronic acid can also be used, and can have the effect of promoting sustained duration in the circulation.

In certain embodiments, the compositions can be selected for inhalation or for delivery through the digestive tract, such as orally. Compositions for oral administration include powders or granules, suspension or solutions in water or non-aqueous media, capsules, sachets, or tables. Thickeners, flavorings, diluents, emulsifiers, dispersing aids or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8. 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5.

In certain embodiments, a pharmaceutical composition can comprise a therapeutically effective amount of a modified nucleic acid in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving a modified nucleic acid in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998043615, Calo, E. et al. (2015) Eur. Polymer J 65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15: 167-277; and Langer (1982) Chem Tech 12:98-105), ethylene vinyl acetate (Hsu and Langer (1985) J Biomed Materials Res 19(4):445-460) or poly-D(−)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In still another aspect, unit dose forms comprising a modified nucleic acid as described in this disclosure are provided. A unit dose form can be formulated for administration according to any of the routes described in this disclosure. In one example, the unit dose form is formulated for intravenous or intraperitoneal administration. In still another aspect, pharmaceutical packages comprising unit dose forms of a modified nucleic acid are provided.

The modified nucleic acids disclosed herein are suited for the preparation of a kit. In some embodiments, kits are provided for carrying out any of the methods described herein. The kits of this disclosure may comprise a carrier container being compartmentalized to receive in close confinement one or more containers such as vials, tubes, syringes, and the like, each of the containers comprising one of the separate elements to be used in the method.

A modified nucleic acid as described in this disclosure for use in treating a subject may be delivered in a pharmaceutical package or kit to doctors and subjects. Such packaging is intended to improve patient convenience and compliance with the treatment plan. Typically the packaging comprises paper (cardboard) or plastic. In some embodiments, the kit or pharmaceutical package further comprises instructions for use (e.g., for administering according to a method as described herein).

In one embodiment, the kit or pharmaceutical package comprises a modified nucleic acid in a defined, therapeutically effective dose in a single unit dosage form or as separate unit doses. The dose and form of the unit dose (e.g., tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### V. Methods of Treatment

As described herein, the present disclosure provides a method of treating a subject, comprising administering to the subject a therapeutically effective amount of a pharmaceutical preparation comprising a modified nucleic acid described herein. In some embodiments, the pharmaceutical preparation comprises a HULC mimic (e.g., as described above), a Pair mimic (e.g., as described above), or a HULC mimic and a Pair mimic. As demonstrated in the Examples below, the modified nucleic acids promote the enzymatic conversion of phenylalanine to tyrosine by a phenylalanine hydroxylase. In some embodiments, the subject has or is suspected to have PKU or HPA. In some embodiments, the subject has symptoms suggestive of PKU or HPA. In some embodiments, the subject is diagnosed as having PKU or HPA. As described above, PKU and/or HPA can be diagnosed by measuring the concentration of phenylalanine in blood. In some embodiments, the subject is human. The subject can be a neonate, an infant, a child under 12 years of age, or a child over 12 years old or an adult.

In some embodiments, administration of the pharmaceutical preparation comprising a modified nucleic acid described herein results in increased or stimulated enzymatic conversion of phenylalanine to tyrosine by a phenylalanine hydroxylase (PAH) in one or more cells of the subject. In some embodiments, the one or more cells comprise liver cells. In some embodiments, administration of the pharmaceutical preparation results in increased affinity of PAH for a PAH substrate and/or a PAH cofactor. In some embodiments, the PAH substrate is phenylalanine. In some embodiments, the PAH cofactor is tetrahydrobiopterin (BH₄).

In some embodiments, the subject comprises a genetic mutation known to contribute to PKU or HPA. In some embodiments, the mutation is in the gene encoding PAH. In some embodiments, the mutation is homozygous or heterozygous. In some embodiments, the PAH mutation is a missense mutation. In some embodiments, the mutation is R408W (i.e., the arginine at position 408 of the wild-type PAH sequence is substituted with a tryptophan). In some embodiments, the mutation results in protein misfolding and/or impairment of catalytic function. In some embodiments, the subject comprises a heterozygous or homozygous mutation in the DNAJC12 gene.

In some embodiments, the subject to be treated may display one or more symptoms indicative of PKU and/or HPA. Such symptoms include, but are not limited to, any of brain damage, intellectual disability, behavioral issues, seizures, and psychiatric disorders. In some embodiments, the subject may have been receiving standard care and may continue to receive such care during treatment with the modified nucleic acid compositions provided herein. In some instances, the subject is also being treated with standard care such as a Phe-restricted diet, BH₄ supplementation, phenylalanine ammonia lyase (PAL) enzyme supplementation or substitution therapy, and/or gene therapy.

As used herein, “treating” or “treatment” of any disease or disorder refers to preventing or ameliorating a disease or disorder in a subject or a symptom thereof. The term ameliorating refers to any therapeutically beneficial result in the treatment of a disease state, e.g., PKU or HPA, lessening in the severity, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Thus, in the disclosed methods, treatment can refer to a 10%, 20%, 30%, 40%, 50%, 60%, 70%, 80%, 90%, or 100% reduction in the severity of an established disorder or condition or symptom of the disorder or condition. For example, a method for treating a subject with PKU by administering a modified nucleic acid as described in this disclosure is considered to be a treatment if there is a 10% reduction in one or more symptoms of PKU in a subject as compared to a control. Thus, the reduction can be a 10%, 20%, 30%, 40%, 50%, 60%, 70%, 80%, 90%, 100%, or any percent reduction in between 10% and 100% as compared to native or control levels. In some embodiments, modified nucleic acid compositions are administered to the subject until the subject exhibits amelioration of at least one symptom of PKU and/or HPA. It is understood that treatment does not necessarily refer to a cure or complete ablation of the disease, condition, or symptoms of the disease or condition.

“Administering” or “administration of” a composition to a subject (and grammatical equivalents of this phrase), as used herein, refer to the act of injecting or otherwise physically delivering a substance as it exists outside the body (e.g., a modified nucleic acid provided herein or a pharmaceutical composition comprising a modified nucleic acid) into a subject. Administration can be via enteral or parenteral routes. In some embodiments, administration is by mucosal, intradermal, intravenous, intramuscular, subcutaneous delivery and/or any other method of physical delivery described herein or known in the art. In some embodiments, the pharmaceutical preparation described herein is administered via intravenous injection, subcutaneous injection, and/or intraperitoneal injection. When a disease, or a symptom thereof, is being treated, administration of the substance typically occurs after the onset of the disease or symptoms thereof. When a disease, or symptoms thereof, are being prevented, delayed, or reduced in severity, administration of the substance typically occurs before the onset of the disease or symptoms thereof. Administration refers to direct administration, which may be administration to a subject by a medical professional or may be self-administration, and/or indirect administration, which may be the act of prescribing a composition. For example, a physician who instructs a subject to self-administer a composition and/or provides a subject with a prescription for a composition is administering the composition to the subject.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing polypeptides are well known to those of skill in the art. Generally, such systems should utilize components that will not significantly impair the biological properties of the modified nucleic acids, such as the capacity to interact with a PAH enzyme (see, for example, Remington's Pharmaceutical Sciences, 18th edition, 1990, Mack Publishing). Those of skill in the art can readily determine the various parameters and conditions for producing modified nucleic acid injectates or infusates without resorting to undue experimentation.

As used herein, the term “therapeutically effective amount” refers to an amount of modified nucleic acid composition as described herein that, when administered to a subject, is effective to achieve an intended purpose, e.g., to reduce or ameliorate at least one symptom of PKU and/or HPA. In some embodiments, administration of a therapeutically effective amount results in a reduction in the level of phenylalanine in the blood. In some embodiments, administration of a therapeutically effective amount results in a blood phenylalanine level of less than 900 μmol/L (e.g., less than 800 μmol/L, less than 700 μmol/L, less than 600 μmol/L, less than 500 μmol/L, less than 400 μmol/L, less than 360 μmol/L, less than 300 μmol/L, less than 250 μmol/L, less than 200 μmol/L, less than 175 μmol/L, less than 150 μmol/L, or less than 125 μmol/L). In some embodiments, administration of a therapeutically effective amount results in a blood phenylalanine level within the range of around 120-360 μmol/L. A therapeutically effective amount is not, however, a dosage so large as to cause adverse side effects, such as unwanted build-up of modified nucleic acid in a tissue or organ of the subject. A therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics or treatments that are administered to the subject. Although individual needs may vary, determination of optimal ranges for effective amounts of formulations is within the skill of the art. Human doses can be extrapolated from animal studies. Generally, the dosage required to provide an effective amount of a formulation, which can be adjusted by one skilled in the art, will vary depending on the age, health, physical condition, weight, type and extent of the disease or disorder of the recipient, frequency of treatment, the nature of concurrent therapy (if any), the method of administration, and the nature and scope of the desired effect(s) (Nies et ah, Chapter 3 In: Goodman & Gilman's The Pharmacological Basis of Therapeutics, 9th Ed., Hardman et ah, eds., McGraw-Hill, New York, NY, 1996). It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication. In some instances, a therapeutically effective amount may vary from about 0.01 mg/kg to about 50 mg/kg, preferably from about 0.1 mg/kg to about 20 mg/kg, most preferably from about 0.2 mg/kg to about 10 mg/kg, in one or more dose administrations daily, for one or several days.

In some embodiments, the modified nucleic acid composition is administered to the subject at least once a day, at least twice a day, or at least three times a day. In some embodiments, the modified nucleic acid composition is administered to the subject at least 1 day, 2, days, 3, days, 4 days, 5 days, 6 days, or 7 days per week. In some embodiments, the modified nucleic acid composition is administered on consecutive days or on non-consecutive days. In some instances, the modified nucleic acid composition is administered to the subject for at least 1 day, at least 2 days, at least 4 days, at least 5 days, at least 6 days, at least 1 week, at least 2 weeks, at least 3 weeks, at least 1 month, at least 2 months, or at least 3 months. In some instances, the nucleic acid composition is administered to the subject on a long term basis for as long as the treatment continues to provide therapeutic benefit.

A pharmaceutical preparation as described herein can comprise a therapeutically effective amount of a modified nucleic acid composition described herein. Such effective amounts can be readily determined by one of ordinary skill in the art as described above. Considerations include the effect of the modified nucleic acid, or the combinatorial effect of the modified nucleic acid with one or more additional active agents, if more than one agent is used in or with the pharmaceutical composition.

Suitable human doses of any of the modified nucleic acids described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of the modified nucleic acid compositions described herein can be determined by known pharmaceutical procedures in cell cultures or experimental animals (e.g., animal models of any of the disease states described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A modified nucleic acid composition that exhibits a high therapeutic index is preferred. While compositions that exhibit toxic side effects may be used, care should be taken to design a delivery system that targets such compositions to the desired tissue (e.g., liver tissue) and to minimize potential damage to normal cells and, thereby, reduce side effects.

The data obtained from the cell culture assays and animal studies can be used in formulating a range of dosage for use in humans. The dosage of a modified nucleic acid composition lies generally within a range of circulating concentrations of the modified nucleic acid compositions that includes the ED₅₀ with little or no toxicity. The dosage may vary within this range depending upon the dosage form employed and the route of administration utilized. For modified nucleic acid compositions described herein, the therapeutically effective dose can be estimated initially from cell culture assays. A dose can be formulated in animal models to achieve, e.g., a circulating plasma concentration range that includes the EC₅₀ (i.e., the concentration of the modified nucleic acid that achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

In some embodiments, a modified nucleic acid composition described herein can be administered to a subject as a monotherapy. Alternatively, the modified nucleic acid composition can be administered in conjunction with other therapies for PKU and/or HPA (combination therapy). For example, the composition can be administered to a subject at the same time, prior to, or after, a second therapy. In some embodiments, the modified nucleic acid composition and the one or more additional active agents are administered at the same time. In some embodiments, the modified nucleic acid composition and the one or more additional active agents are administered at different times. In some embodiments, the modified nucleic acid composition and the one or more additional agents can be administered simultaneously by the same route or by different routes. In some embodiments, a composition comprising the modified nucleic acid composition and one or more additional agents is administered to the subject. In certain embodiments, the other therapies may include, for example, dietary restrictions, sapropterin supplementation, gene therapy, enzyme replacement therapy (e.g., using a phenylalanine ammonia lyase fusion protein such as Palynziq® (pegvaliase-pqpz), a modified phenylalanine ammonia lyase enzyme such as CDX-6114 (Codexia), or an mRNA therapy such as mRNA-3283 (Modema)), enzyme substitution therapy, and/or other suitable therapies. e.g., SYNB-1618 (Synlogic), Oxitriptan (also known as 5-hydroxytryptophan (5-HTP)) supplementation, or sepiapterin supplementation (e.g., CNSA-001 (Censa Pharma)).

A modified nucleic acid described herein can replace or augment a previously or currently administered therapy. For example, upon treating with a modified nucleic acid, administration of the one or more additional active agents can cease or be reduced, e.g., be administered at lower levels or dosages. In some embodiments, administration of the previous therapy can be maintained. In some embodiments, a previous therapy is maintained until the level of the modified nucleic acid reaches a level sufficient to provide a therapeutic effect.

Monitoring a subject (e.g., a human patient) for an improvement of a PKU and/or HPA symptom, as defined herein, means evaluating the subject for a change in a disorder parameter, e.g., a reduction in one or more symptoms of PKU and/or HPA exhibited by the subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or eliminate a selected therapeutic modality, e.g., adding or dropping any of the treatments for PKU and/or HPA described herein.

Disclosed herein are materials, compositions, and methods that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are various additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXEMPLARY EMBODIMENTS

Exemplary embodiments provided in accordance with the presently disclosed subject matter include, but are not limited to, the claims and the following embodiments:

Embodiment 1 is a modified nucleic acid comprising: a) a nucleotide sequence having at least 80% identity to any one of SEQ ID NOs: 3-7; and b) at least one nucleotide comprising a 2′-fluoro base modification.

Embodiment 2 is the modified nucleic acid of embodiment 1, wherein the nucleotide sequence has at least 80% identity to any one of SEQ ID NOs: 5-7.

Embodiment 3 is the modified nucleic acid of embodiment 1 or 2, wherein the nucleotide sequence has at least 80% identity to SEQ ID NO:6.

Embodiment 4 is the modified nucleic acid of any one of embodiments 1 to 3, wherein the modified nucleic acid has a length of 15 to 40 nucleotides.

Embodiment 5 is the modified nucleic acid of any one of embodiments 1 to 4, wherein at least one of the nucleotides of the modified nucleic acid is a ribonucleotide.

Embodiment 6 is the modified nucleic acid of any one of embodiments 1 to 5, wherein a majority of the nucleotides of the modified nucleic acid are ribonucleotides.

Embodiment 7 is the modified nucleic acid of any one of embodiments 1 to 6, wherein at least 25% of the nucleotides comprise a 2′-fluoro base modification.

Embodiment 8 is the modified nucleic acid of any one of embodiments 1 to 7, wherein all of the internal nucleotides comprise a 2′-fluoro base modification.

Embodiment 9 is the modified nucleic acid of any one of embodiments 1 to 8, wherein the modified nucleic acid comprises at least one phosphorothioate bond.

Embodiment 10 is the modified nucleic acid of any one of embodiments 1 to 9, wherein the 5′ terminal nucleotide and/or the 3′ terminal nucleotide is attached via phosphorothioate bond.

Embodiment 11 is the modified nucleic acid of any one of embodiments 1 to 10, wherein the modified nucleic acid comprises a tag attached to the 3′ terminal nucleotide and/or the 5′ terminal nucleotide.

Embodiment 12 is the modified nucleic acid of embodiment 11, wherein the tag comprises an apolipoprotein E peptide.

Embodiment 13 is the modified nucleic acid of embodiment 12, wherein the apolipoprotein E peptide comprises an amino acid sequence having at least 90% identity to SEQ ID NO:9.

Embodiment 14 is the modified nucleic acid of embodiment 11, wherein the tag comprises N-acetylgalactosamine.

Embodiment 15 is the modified nucleic acid of any one of embodiments 1 to 14, wherein the modified nucleic acid increases the affinity of a phenylalanine hydroxylase (PAH) for a PAH substrate and/or a PAH cofactor.

Embodiment 16 is a nanoparticle comprising the modified nucleic acid of any one of embodiments 1 to 15.

Embodiment 17 is a pharmaceutical preparation comprising: (a) the modified nucleic acid of any one of embodiments 1 to 15 or the nanoparticle of embodiment 16; and (b) a pharmaceutically acceptable carrier.

Embodiment 18 is a method for treating a subject diagnosed with or suspected to have phenylketonuria or hyperphenylalaninaemia, the method comprising administering to the subject a therapeutically effective amount of the pharmaceutical preparation of embodiment 17.

Embodiment 19 is the method of embodiment 18, wherein the subject has been diagnosed with phenylketonuria.

Embodiment 20 is the method of embodiment 18 or 19, wherein the subject has been diagnosed with hyperphenylalaninaemia.

Embodiment 21 is the method of any one of embodiments 18 to 20, wherein the subject has symptoms suggestive of phenylketonuria or hyperphenylalaninaemia.

Embodiment 22 is the method of any one of embodiments 18 to 21, wherein the subject has a mutation in a phenylalanine hydroxylase gene.

Embodiment 23 is the method of embodiment 22, wherein the mutation is R408W.

Embodiment 24 is the method of any one of embodiments 18 to 23, wherein the pharmaceutical preparation is administered via intravenous injection, subcutaneous injection, and/or intraperitoneal injection.

Embodiment 25 is the method of any one of embodiments 18 to 24, wherein administration of the pharmaceutical preparation results in increased enzymatic conversion of phenylalanine to tyrosine by a phenylalanine hydroxylase (PAH) in one or more cells of the subject.

Embodiment 26 is the method of any one of embodiments 18 to 25, wherein administration of the pharmaceutical preparation results in increased affinity of phenylalanine hydroxylase (PAH) for a PAH substrate and/or a PAH cofactor.

Embodiment 27 is the method of embodiment 26, wherein the PAH substrate is phenylalanine.

Embodiment 28 is the method of embodiment 26 or 27, wherein the PAH cofactor is tetrahydrobiopterin (BH4).

### EXAMPLES

The following examples are offered to illustrate, but not to limit, the claimed invention. Many of the following examples are further described in Li et al., 2021, “A noncoding RNA modulator potentiates phenylalanine metabolism in mice,” Science 373(6555):662-673, which is hereby incorporated by reference in its entirety. Reference is made to this Li et al. 2021 publication for illustration of certain experimental data as described in the instant disclosure.

### Example 1. Depletion of lncRNAs Drives PKU

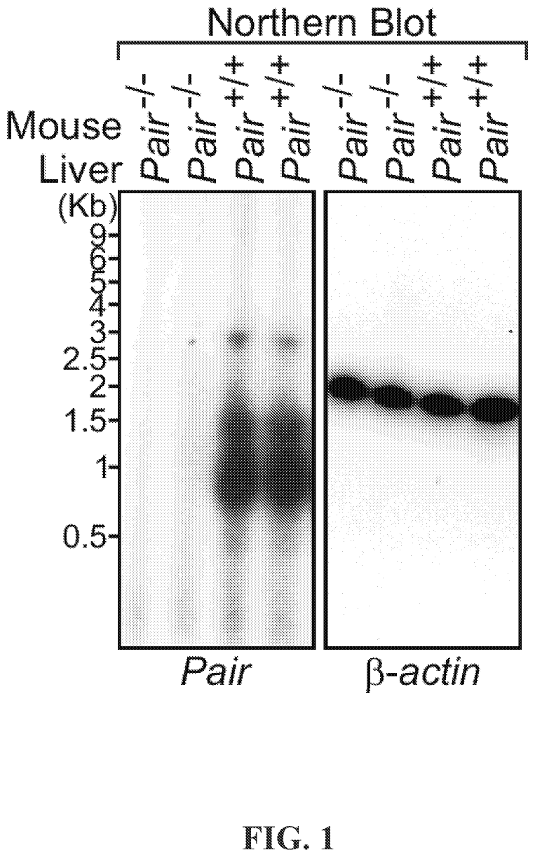

Long intergenic noncoding RNAs (lincRNAs) and long non-coding RNAs (lncRNAs) are transcripts with low coding potential. The lncRNA profile of mouse E18.5 embryos and the livers of 2-month-old adult mice were determined to investigate the biological importance of lncRNAs (data not shown; see FIGS. 1A and S1A-S1C in Li et al. 2021), and it was found that 2210408F21Rik (NR 040259) (renamed Pair: PAH-activating lincRNA) is one of the most upregulated lncRNAs in the liver of adults compared to the E18.5 embryos and exhibits low coding potential (CNIT score (˜0.3544) (6) (data not shown; see FIGS. S1D-S1H in Li et al. 2021). To deplete this mouse lncRNA, a two-nucleotide mutation was introduced using CRISPR/Cas9 to interrupt the splicing site after the first exon (data not shown; see FIG. 1B in Li et al. 2021). Northern blotting indicated that Pair exhibits two major isoforms with molecular weights 730 bp and 1.5 kb, which were both depleted upon the introduction of the splicing-site mutation (FIG. 1). Compared to wild-type and heterozygous littermates, Pair^{-/-} mice exhibited similar expression of neighboring genes with no detectable alterations in major organ development (data not shown; see FIGS. S2A-S2D in Li et al. 2021).

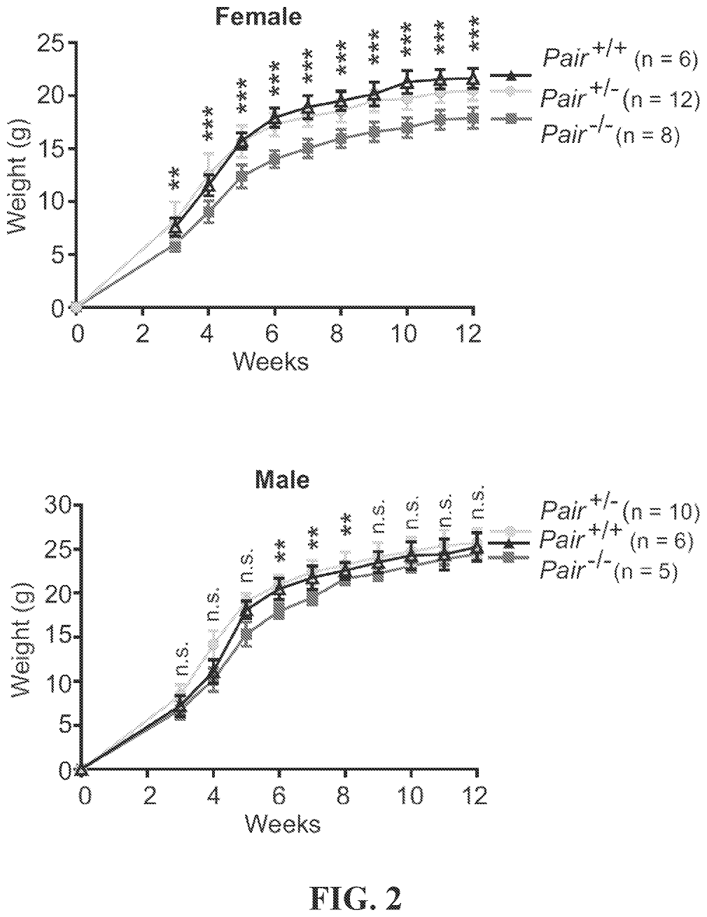

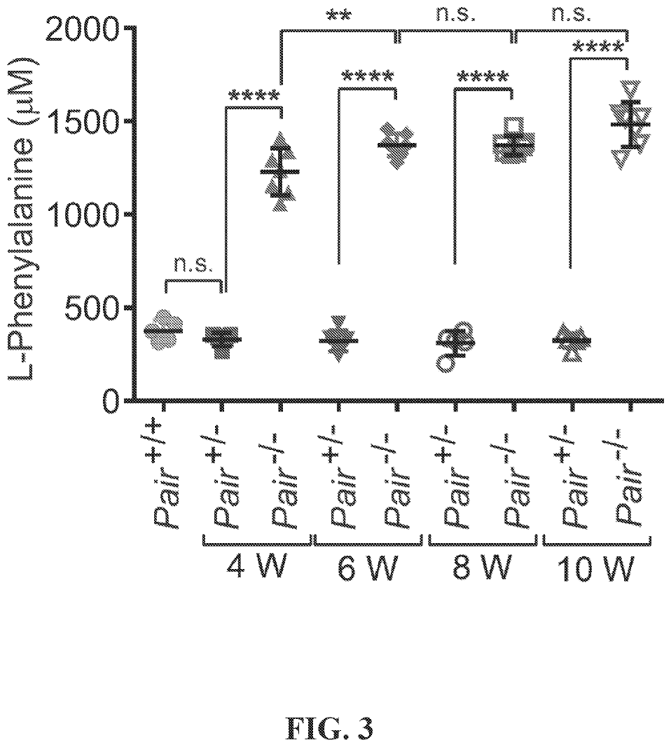

Both male and female Pair^{-/-} mice exhibited hypopigmentation (data not shown; see FIG. 1D in Li et al. 2021), growth retardation and elevated serum Phe concentrations (FIG. 2 and FIG. 3) reminiscent of human PKU. Pair^{-/-} livers showed no detectable changes in the expression of BH₄ biogenesis genes or PAH protein abundance (data not shown; see FIGS. S2E-S2G in Li et al. 2021). Pair^{-/-} livers exhibited enzymatic deficiency in converting Phe to Tyr, and Pair^{+/-} livers showed impaired PAH enzymatic activity (data not shown; see FIGS. S2H-S21 in Li et al. 2021). Pair^{+/-} mice exhibited blood Phe concentrations within the normal range; however, these animals showed elevated blood Phe concentrations upon Phe challenge compared to Pair^{+/+} mice (data not shown; see FIG. S2J in Li et al. 2021). These results suggested that Pair^{+/-} livers exhibit partial PAH deficiency. Sanger sequencing of the Pah gene suggested that Pah^{enu2} mice harbor a T788C mutation, while Pair^{-/-} mice harbor a wild-type Pah gene (data not shown; see FIGS. S2K-S2L in Li et al. 2021).

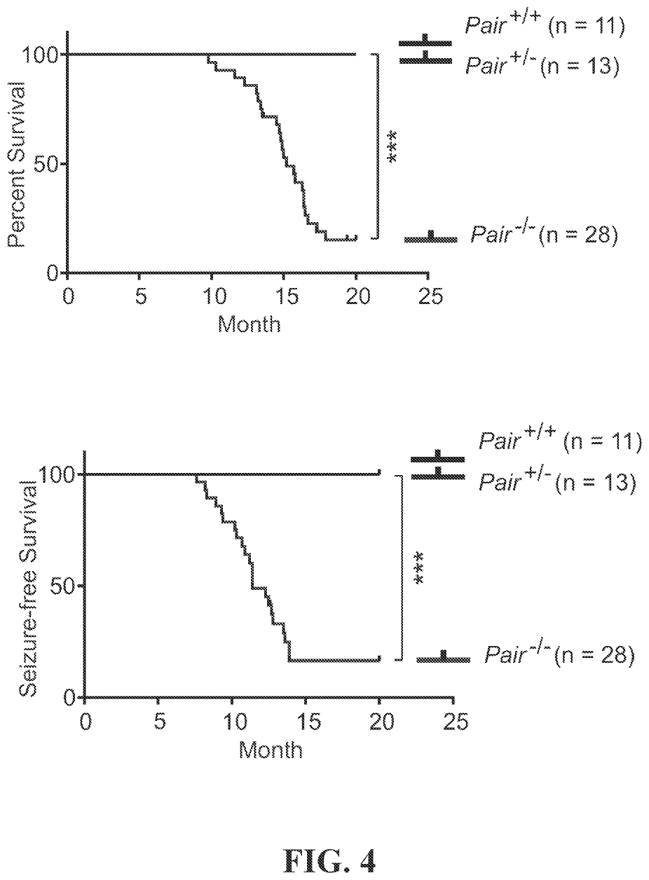

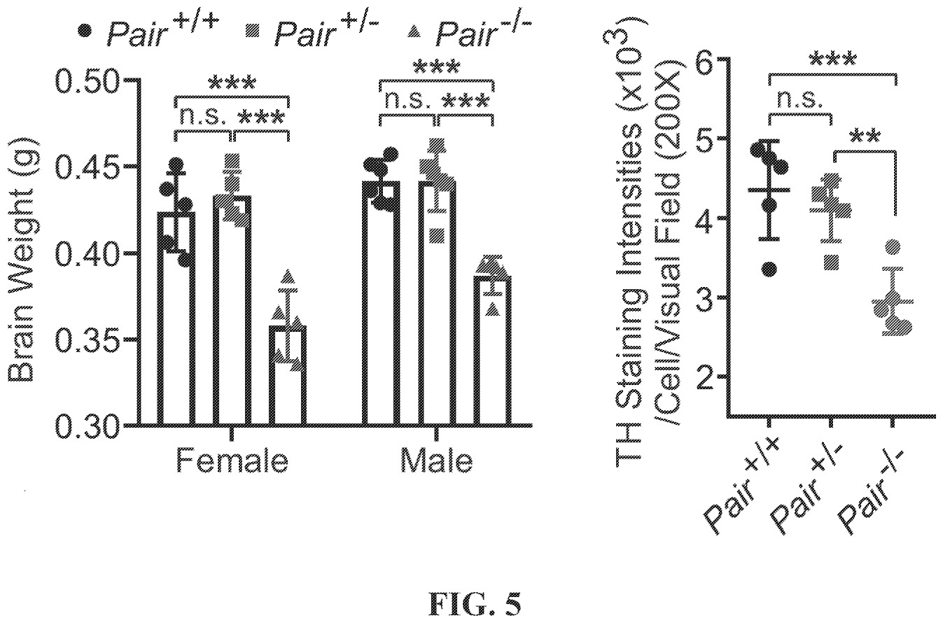

The median life-span of Pair^{-/-} mice is 15.2 months (FIG. 4), and more than 70% of the mice exhibited seizures starting at the ages of 8-10 months (FIG. 4 and see FIG. 1J in Li et al. 2021). Following cardiopulmonary resuscitation (CPR), Pair KO mice experiencing a seizure can be rescued (data not shown). Pair^{-/-} brains were smaller and exhibited reduced tyrosine hydroxylase (TH)-positive neurons compared to wild-type and heterozygous littermates (FIG. 5 and see FIGS. 1K and 1L in Li et al. 2021). Pair^{-/-} livers and serum exhibited diminished tyrosine concentrations. Tyrosine is catalytically produced by PAH in both serum and liver tissue (data not shown; see FIGS. S3A-S3E in Li et al. 2021), confirming the presence of PAH deficiency. The role of Pair in seizures, in addition to regulating PAH enzymatic activities, could not be ruled out. The data suggested that Pair^{-/-} mice model human PKU.

### Example 2. Pair and HULC Associate with PAH

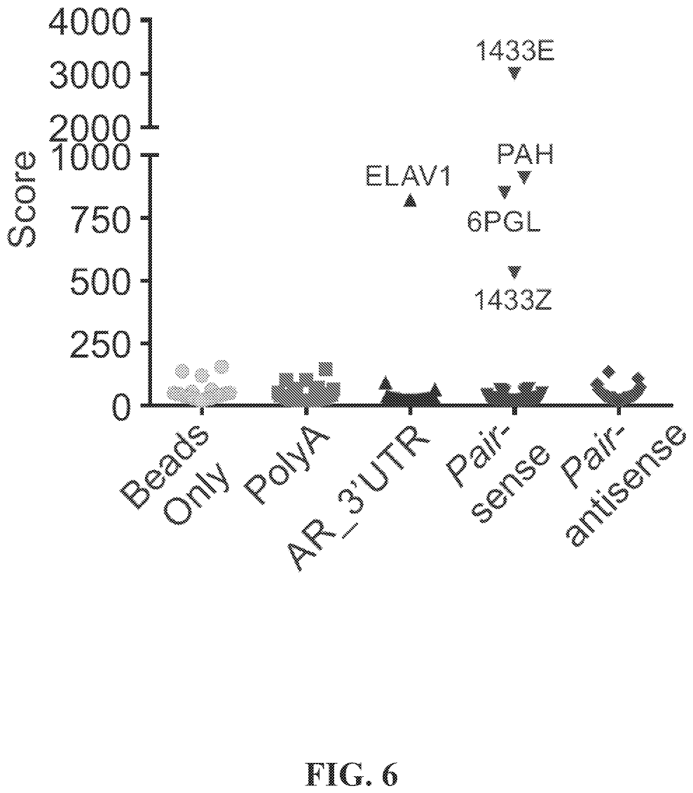

To understand the molecular mechanism of Pair, Pair pulldown was performed using biotinylated sense or anti-sense Pair in mouse livers (FIG. 6). Sense, but not antisense Pair, associated with mouse PAH (FIG. 6). Streptavidin beads-only and polyadenosine RNA were used as negative controls, while the association of ELAV1 with AR_3′ UTR served as a positive control (7) (FIG. 6). CLIP (cross-linking immuno-precipitation) assays were performed using mouse Pair^{+/+} and Pair^{-/-} livers or human liver tissues from two healthy donors (Table 2 and see FIGS. S4A and S4B in Li et al. 2021). The presence of PAH-RNA complexes was diminished upon Pair knockout (data not shown; see FIG. S4A in Li et al. 2021). PAH-lncRNA(s) complexes were detected in the two human liver donors (data not shown; see FIG. S4B in Li et al. 2021). The PAH-RNA complexes were subjected to reverse transcription and Sanger sequencing (data not shown). As expected, mouse PAH associated with mouse Pair (nt. 460-496) in Pair^{+/+} livers (data not shown; see FIG. 2B in Li et al. 2021, bottom row). Human PAH associated with one human lncRNA gene, HULC (nt. 183-216) (data not shown; see FIG. 2B in Li et al. 2021, top row). Although HULC has been suggested to be upregulated in liver cancer (8), northern blotting indicated that HULC, similarly to Pair, is specifically expressed in normal liver tissues (data not shown; see FIGS. S4C-S4E in Li et al. 2021), suggesting the biological relevance of HULC in liver homeostasis and function.

TABLE 2
Clinical characteristics of tissue samples.

| Sample | | | | Sample | | | Date | Cause | PMI, | Tissue |
|---|---|---|---|---|---|---|---|---|---|---|
| ID | Sex | Age | Ethnicity | type | Matrix | Diagnosis | of autopsy | of death | hours | weight, g |
| 09899A(4) | M | 56 | Caucasian | FF | liver | normal | Nov. 16, 2015 | acute cardiac | 1.5 | 0.8 |
| | | | | | | tissue | | insufficiency | | |
| 09738A(14) | M | 33 | Caucasian | FF | liver | normal | Aug. 20, 2014 | aortic aneurism | 1.5 | 1 |
| | | | | | | tissue | | rupture, cardiac | | |
| | | | | | | | | tamponade | | |

RNA immunoprecipitation (RIP) assay confirmed that Pair and HULC associate with PAH protein in mouse and human livers, respectively (data not shown; see FIG. S5A-S5C in Li et al. 2021). The regulatory (aa. 1-142) and catalytic (aa. 142-411) domains of PAH are required for PAH-Pair interactions (data not shown; see FIGS. S5D-S5G in Li et al. 2021). Next, using primary cultured mouse or human hepatocytes, it was demonstrated that PAH protein faithfully co-localizes with Pair or HULC in the cytosol, but not with another cytosolic lncRNA, Tug1 (data not shown; see FIGS. 2C and S5H-S51 in Li et al. 2021).

Each nucleotide between Pair (nt. 460-496) and HULC (nt. 183-216) was substituted using the most common transition and transversion types (9) and wild-type or mutant Pair or HULC were expressed in Pair^{-/-} or HULC-deficient hepatocytes (data not shown; see FIGS. S6A and S6B in Li et al. 2021). RIP assay suggested that Pair (nt. 470-488) and HULC (nt. 183-200) are required for PAH-Pair and PAH-HULC interactions. Furthermore, Pair 479A>G and HULC 191A>G abolished PAH-Pair and PAH-HULC interactions, respectively.

SHAPE assays (10, 11) indicated that HULC U190 and A191 (capillary electrophoresis size #290 and 289) exhibited chemical labeling, suggesting the presence of a loop structure flanked by low chemical probing (data not shown; see FIGS. 2D and S6C in Li et al. 2021). Furthermore, HULC harbors 3 additional stem-loop structures, as revealed by SHAPE assays (data not shown; see FIGS. S7A-S7D in Li et al. 2021). These 3 stem-loop structures exhibited distinct structures compared to the stem-loop at nt. 184-216 (data not shown; see FIGS. S7E-S7H in Li et al. 2021). The chemical labeling of HULC^{A191} was verified by in vivo SHAPE (data not shown; see FIGS. S8A and S8B in Li et al. 2021). The secondary structure of Pair was also analyzed, and it was found that Pair nt. 467-484 also exhibits a stem-loop structure, with A479 showing robust chemical probing (data not shown; see FIGS. S9A-S9C in Li et al. 2021). Pair nt. 467-484 exhibited a similar 3-dimensional structure to HULC nt. 184-216 but not the above 3 stem-loops of HULC (data not shown; see FIG. S9D in Li et al. 2021).

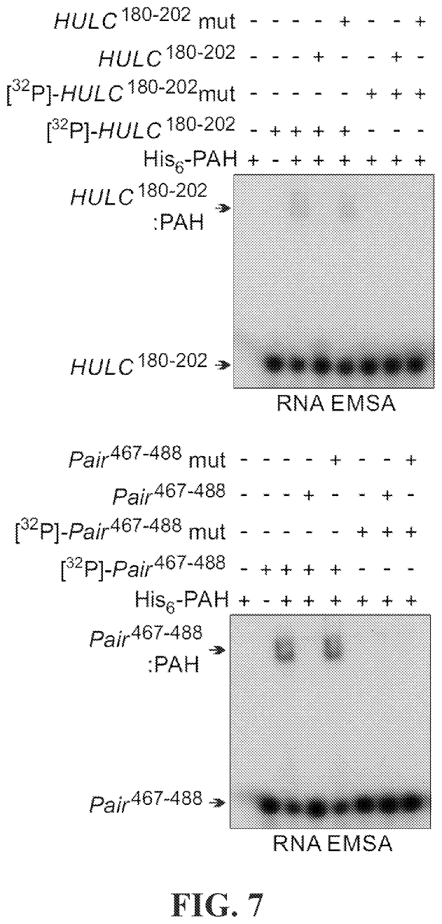

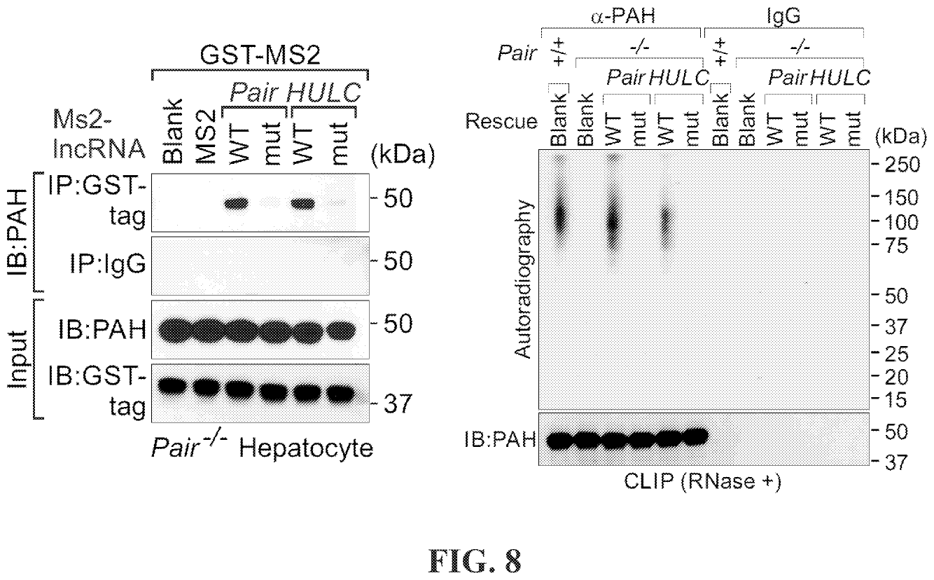

Wild-type Pair and wild-type HULC, but not the Pair 479A>G or HULC 191A>G mutants (referred to as Pair mut or HULC mut, respectively) associated with recombinant PAH, as revealed by RNA electrophoretic mobility shift assay (EMSA) assay (FIG. 7). Expression of MS2-tagged wild-type Pair HULC, but not the mutants, rescued the association with PAH protein in Pair^{-/-} hepatocytes (FIG. 8 and see FIG. S10A in Li et al. 2021). A rescue CLIP assay was performed by expressing exogenous wild-type Pair HULC or mutants in mouse Pair^{-/-} hepatocytes (FIG. 8 and see FIG. S10B in Li et al. 2021). Exogenous Pair or HULC WT, but not mutants, associated with PAH similarly to endogenous Pair or HULC (FIG. 8). Similar amounts of PAH protein were immuno-precipitated using anti-PAH antibody (FIG. 8, bottom). These findings suggested that the human lncRNA HULC and mouse Pair both associate with PAH.

### Example 3. HULC/Pair Modulate the Enzymatic Activity of PAH

To demonstrate the underlying molecular mechanisms of HULC/Pair in the enzymatic activity of PAH, it was first determined that there are roughly 800 HULC RNA, 700 Pair RNA, and 4,000 PAH protein molecules per human or mouse hepatocyte (data not shown; see FIGS. S10C-S10E in Li et al. 2021). In Pair^{-/-} or HULC-deficient hepatocytes, exogenous expression of Pair HULC in a dose-dependent manner led to the reduction of cellular Phe concentrations (data not shown; see FIGS. S10F and S10G in Li et al. 2021). The concentrations of amino acids other than Phe and Tyr were minimally affected upon Pair depletion (data not shown; see FIG. S10H in Li et al. 2021).

The enzymatic activity of PAH requires the presence of a cofactor, BH₄ (12). Mutations of PAH affecting PAH-Phe or PAH-BH₄ interactions impair the catalytic activity of PAH (13). Previous research indicated that the Phe molecule associates with the regulatory domain of PAH as an allosteric activator (14, 15, 16). Using the crystal structure information of PAH (PDB 6HYC), protein structural bioinformatics analysis suggested that HULC nt. 184-216 associates with the regulatory domain of PAH and allosteric Phe (see FIG. 3A in Li et al. 2021). HULC A¹⁹¹ forms hydrogen bonds with both Thr63 and His64 (see FIG. 3A in Li et al. 2021). The mutation of A¹⁹¹ to G¹⁹¹ causes a change at position 6 from an amino group (6-NH₂) to a carbonyl group (6-CO). This change leads to the loss of the hydrogen bond between A¹⁹¹ and His64, because carbonyl groups are hydrogen bond acceptors while amino groups are often hydrogen bond donors. The amino group (2-NH₂) of G¹⁹¹ may also cause steric hindrance with Thr63. Phe forms hydrogen bonds with PAH via Asn61 and Leu62, and it also forms stacking interactions with A¹⁹¹ (see FIG. 3B in Li et al. 2021). These interactions stabilize the whole structural complex in a conformation that makes the active site fully accessible to Phe as a substrate and BH₄ as a cofactor. These findings suggested that HULC serves as an important factor that stabilizes the interaction between allosteric Phe and PAH, as previously hypothesized (14). Aside from A¹⁹¹, HULC interacts with PAH via several other residues to achieve the binding specificity of the PAH-HULC interaction: A¹⁹⁵ forms a hydrogen bond with Tyr166; A²¹⁴ forms a hydrogen bond with Arg157; and G²⁰² also interacts with Tyr154 via a hydrogen bond (see FIG. 3C in Li et al. 2021). Pair adapts a binding mode to PAH that is similar to HULC: both Pair and HULC have two nucleotides that stick out to form a T-shape and exhibit stacking interactions with His64 of PAH, and these two nucleotides (A and U) stabilize each other through stacking interactions (see FIGS. S101 and 3D in Li et al. 2021). The only difference between Pair and HULC with regard to this behavior is that the order of the sequences of these two nucleotides in Pair is UA; in HULC, the order is AU (see FIG. 3D in Li et al. 2021).

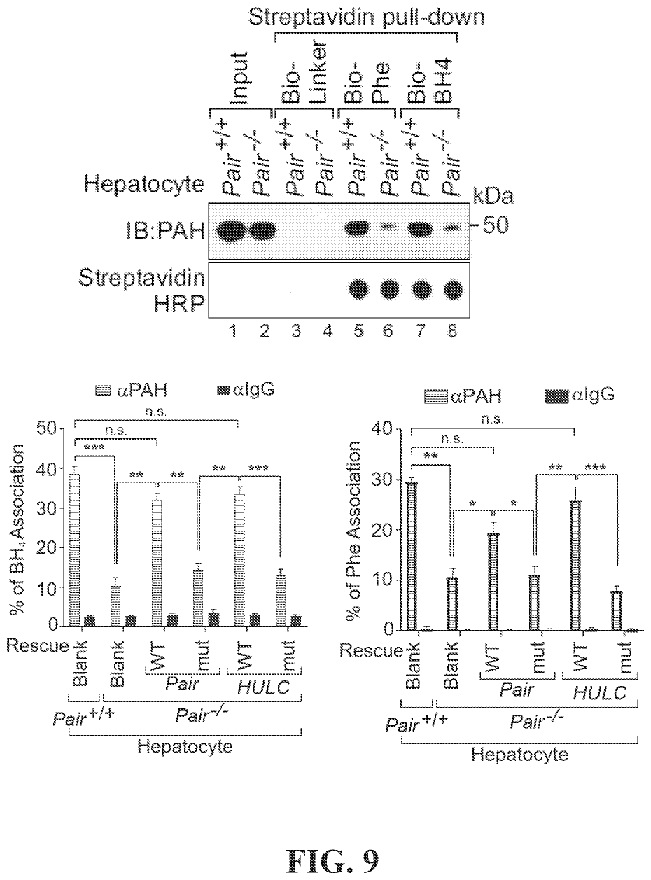

It was reasoned that the HULC-PAH interaction may facilitate the binding of PAH to Phe or BH₄. To address this hypothesis, biotinylated-Phe and -BH₄ (referred to as Bio-Phe and Bio-BH₄) were synthesized (data not shown; see FIG. S11A in Li et al. 2021). Compared to Pair^{+/+} livers, Pair^{-/-} livers exhibited a similar abundance of PAH protein; however, the PAH-Phe and PAH-BH₄ interactions were impaired following Pair depletion (FIG. 9). It was then determined that digoxin (DIG)-tagged HULC showed detectable interactions with bacterially-expressed PAH proteins but undetectable associations with Bio-Phe or Bio-BH₄ (data not shown; see FIG. S11B in Li et al. 2021). Wild-type PAH was included as a positive control. PAH G46S, F55L, and P281L have been suggested to impair interactions between Bio-Phe and/or Bio-BH₄ (17, 18), which was confirmed. LncRNA LINK-A and the interaction between LINK-A and PIP₃ (19) were included as negative controls.

The N-terminal regulatory domain of PAH can undergo a conformational change to switch between “open” and “closed” states (18). It was hypothesized that the binding of HULC may stabilize the PAH-Phe-HULC complex and stabilize the PAH protein in the “open” state (data not shown; see FIG. S11C in Li et al. 2021). Limited proteolysis (LiP) was applied, followed by liquid chromatography-mass spectrometry (LC-MS) analysis (LiP-LC-MS) (20), to address this hypothesis. Open loop regions (gray) and Lip-resistant regions (dark blue) were determined (data not shown; see FIG. 3F in Li et al. 2021). Region aa 57-66 showed resistance to LiP in the presence of HULC but not the HULC mut, suggesting that this region associates with HULC (see FIG. 3F in Li et al. 2021, top panel-magenta). Notably, aa 57-66 of PAH exhibited recovery in the presence of Phe (see FIG. 3F in Li et al. 2021, bottom panel-magenta), which was consistent with the structural modeling, demonstrating that Thr63 and His64 associate with HULC and Phe as a complex. Furthermore, LiP-MS suggested a few regions that were dynamically regulated upon HULC binding (see FIG. 3F in Li et al. 2021, green).

Hence, the data suggested that HULC associates with PAH in vitro and facilitates the potential conformational change of PAH. Consistent with this notion, expression of wild-type Pair or HULC rescued PAH-Phe and PAH-BH₄ interactions and reversed cellular Phe accumulation, while the Pair and HULC mutants failed to do so (FIG. 9 and see FIGS. S11D and S11E in Li et al. 2021).

To determine the functional role of the HULC-PAH interaction in vivo, HULC-deficient hiPSC were further differentiated into hepatocytes expressing wild-type HULC or the A191G mutant (data not shown; see FIG. S12A in Li et al. 2021). Expression of wild-type HULC or the A191G mutant showed no detectable effects on the abundance of PAH protein (data not shown; see FIG. S12B in Li et al. 2021). Upon depletion of HULC, the conversion of ¹⁴C-Phe to ¹⁴C-Tyr was reduced with concurrent elevation of cellular Phe concentrations (data not shown; see FIGS. S12C and S12D in Li et al. 2021). Expression of WT HULC, but not the A191G mutant, restored the enzymatic activity of PAH and reversed the cellular accumulation of Phe. Similarly, wild-type PAH, but not the TH63-64PN mutant, expressed in PAH-deficient hiPSC-hepatocytes rescued the enzymatic deficiency of PAH (data not shown; see FIGS. S12E-512H in Li et al. 2021). Taken together, these findings suggested that Pair-PAH and HULC-PAH interactions facilitate the PAH-driven catalysis of Phe to Tyr.

### Example 4. HULC Mimics Restore PAH Enzymatic Activity

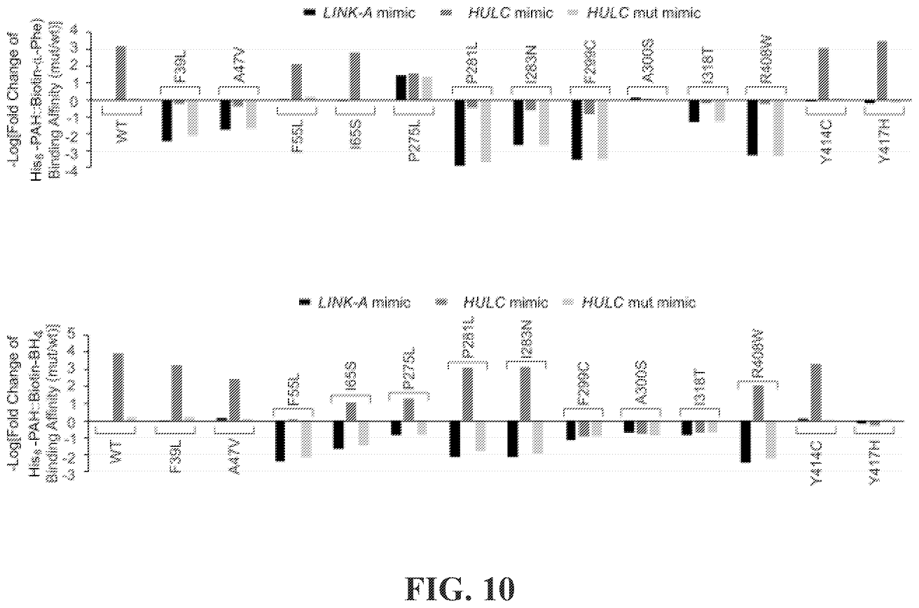

It was reasoned that supplementing lncRNAs might improve the catalytic activities of the PAH mutants, leading to reduced serum Phe concentrations and improved symptoms in patients with PKU. Scramble (Scr) and HULC mimics representing wild-type HULC nt. 181-201 and HULC A191G mutated sequences were designed for the following studies. 17 PAH mutants that were identified from patients with PKU and are known to affect the enzymatic activity of PAH (21, 22) were selected, and bacterially-expressed wild-type and mutant PAH (FIG. 10 and see FIG. S13A in Li et al. 2021) were collected. EMSA assay indicated that 13 of the 17 PAH mutants, as well as wild-type PAH, associated with the HULC mimics; on the contrary, PAH TH63-64PN, R157N, N207S, and S349L failed to associate with the HULC mimics (data not shown; see FIG. S13B in Li et al. 2021). The denatured wild-type PAH proteins were included as a negative control. The interactions between PAH proteins (WT/mutants) and HULC mimics (WT) were further quantified, showing that PAH protein interacted with the HULC mimics with a K_{d} value of 131.6 nM (data not shown; see FIG. S13C in Li et al. 2021). PAH TH63-64PN, R157N, N207S, and S349L, but not the other PAH mutants, exhibited decreased binding affinities.

The binding affinities between PAH (WT/mutants) and Bio-Phe or Bio-BH₄ in the presence of HULC mimics were measured (data not shown; see FIGS. S14 and S15 in Li et al. 2021) and the change in binding affinities compared with PAH WT is summarized in FIG. 10. LncRNA mimics representing LINK-A 1100-1117 (19) were included as a negative control (FIG. 10). For wild-type PAH protein, the presence of HULC mimics enhanced PAH-Phe (FIG. 10, top panel) and PAH-BH₄ interactions (FIG. 10, bottom panel), while HULC mut mimics failed to do so (FIG. 10 and see FIGS. S14 and S15 in Li et al. 2021). The PAH mutants tested all exhibited impaired binding affinities toward Phe and/or BH₄ compared to wild-type PAH protein (FIG. 10 and see FIGS. S14 and S15 in Li et al. 2021). The presence of HULC mimics, but not HULC mut, enhanced the affinity of Phe and/or BH₄ to the PAH mutants. A PAH catalytic pocket deletion mutant (A245-379) was included as a negative control (data not shown; see FIGS. S140 and S150 in Li et al. 2021).

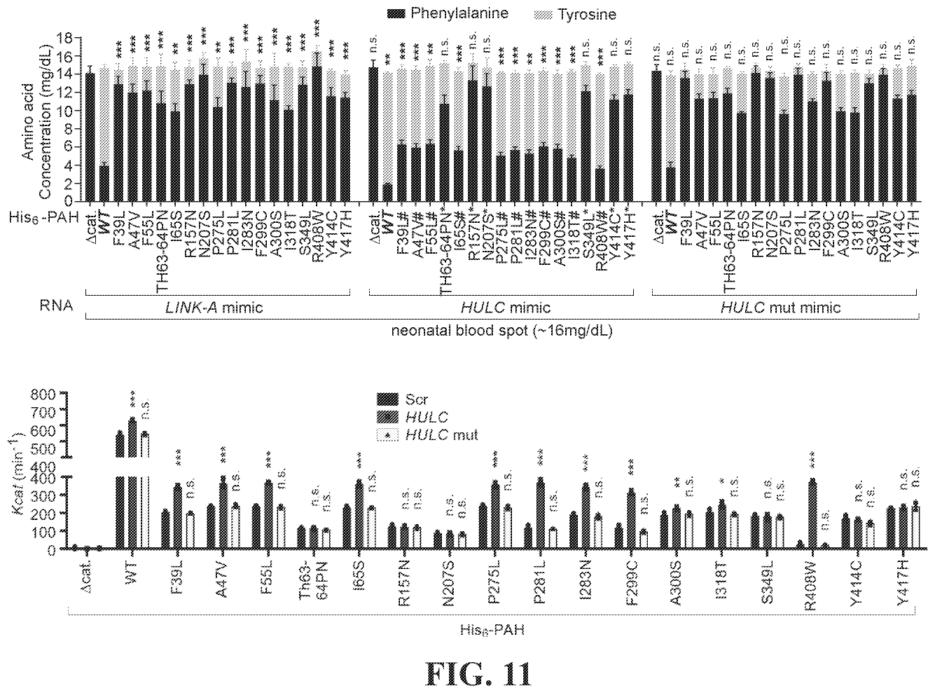

It was further demonstrated that wild-type PAH effectively catalyzed Phe to Tyr, which was abolished in all 17 PAH mutants tested (FIG. 11, top panel, left section, bar #2). In the presence of HULC mimics, but not LINK-A mimics or HULC mut, wild-type PAH showed enhanced enzymatic activity in converting Phe to Tyr (FIG. 11, top panel, middle section, bar #2). Furthermore, 11 of the 17 PAH mutants also showed improved enzymatic activities in converting Phe to Tyr (FIG. 11, top panel, middle section, as indicated with #symbols). The enzymatic activities of the rest of the PAH mutants were not significantly affected by HULC mimics (FIG. 11, top panel, middle section, as indicated with * symbols).

The k_{cat} of bacterially-expressed human wild-type and mutant PAH in the presence of Scramble (Scr), wild-type HULC, or mutant HULC mimics was determined (FIG. 11, bottom panel and see FIG. S16 in Li et al. 2021). Recombinant PAH proteins with deleted catalytic domains (Δcat) were included as a negative control. In the presence of the wild-type HULC mimics, the k_{cat} of wild-type PAH was increased (FIG. 11, bottom panel and see FIG. S16B in Li et al. 2021). All PAH mutants tested exhibited impaired k_{cat} under identical conditions (FIG. 11, bottom panel and see FIGS. S16C-216S in Li et al. 2021). The presence of wild-type HULC mimics, but not mutant HULC mimics, enhanced the k_{cat} of PAH F39L, A47V, F55L, I65S, P275L, P281L, I283N, F299C, A300S, I318T, and R408W, but not the enzymatic activities of PAH TH63-64PN, R157N, N207S, and S349L (FIG. 11, bottom panel and see FIGS. S16C-216S in Li et al. 2021). Therefore, the data suggested that the presence of HULC facilitates the enzymatic activity of wild-type PAH and a cohort of mutants observed in patients with PKU.

### Example 5. HULC/Pair Enhances the Enzymatic Activity of the PAH R408W Mutant

19.2-73% of patients with PKU harbor a R408W mutant (23, 24), and patients with this mutation respond poorly to currently available treatment options. It was hypothesized that overexpression of HULC/Pair might improve the enzymatic activity of PAH R408W. To address this, a Pah^{R408W/R408W} mouse strain was generated using CRISPR/Cas9 (data not shown; see FIGS. S17A and S17B in Li et al. 2021). Similar to Pair^{-/-} mice, Pah^{R408W/R408W} mice exhibited hyperpigmentation, growth retardation, seizures, and elevated blood Phe concentrations, with no detectable alterations in the protein stability of PAH or the expression of Pair (data not shown; see FIGS. S17C-S17H in Li et al. 2021). These observations provided genetic evidence confirming that Pair HULC and PAH act in a linear pathway. In primary cultured mouse hepatocytes isolated from Pair^{-/-} or Pah^{R408W/R408W} mice, cellular Phe, Tyr, and tryptophan (Trp) concentrations were determined, revealing that expression of wild-type Pair HULC, but not the mutant (data not shown; see FIG. S171 in Li et al. 2021), restored PAH-Phe and PAH-BH4 interactions and cellular Phe and Tyr concentrations, with no effect on Trp status (data not shown; see FIGS. S171-S17L in Li et al. 2021).

Skin fibroblasts were collected from healthy donors who harbored wild-type PAH genes and from a patient with PKU (ID: GM02406) who harbored F299C and R408W mutations. These fibroblasts were reprogrammed into hiPSCs. These hiPSCs were further induced into hepatocytes (data not shown; see FIGS. S18A and S18B in Li et al. 2021). Two hiPSC clones were used, referred to as PKU #7 and PKU #17 (data not shown; see FIGS. S18A-S18C in Li et al. 2021). These hiPSC-derived hepatocytes exhibited similar expression of HULC, PAH, and hepatic markers. The hiPSC-derived hepatocytes derived from both PKU #7 and PKU #17 clones exhibited increased Phe concentrations compared to the control (healthy donor), confirming PAH enzymatic activity deficiency (data not shown; see FIG. S18D in Li et al. 2021). Expression of full-length HULC in these hiPSC-derived hepatocytes reduced Phe concentrations in PKU #7 and PKU #17 hepatocytes (data not shown; see FIGS. S18D and S18E in Li et al. 2021). This suggested that a supply of HULC might enhance the enzymatic activity of PAH in F299C and R408W mutants.

### Example 6. GalNAc-HULC Mimics Improve Phenylalanine Metabolism in Mice

To design lncRNA mimics that could provide therapeutic value in vivo, scramble (Scr), wild-type, and mutant HULC mimics were synthesized using 2′-Fluoro (2′-F) modified RNA monomers to provide nuclease resistance in vivo. The potential secondary structure of wild-type or mutant HULC mimics remained identical (data not shown; see FIG. S19A in Li et al. 2021). Both full-length HULC and the mimics exhibited similar binding affinities for PAH (data not shown; see FIG. S19B in Li et al. 2021). Furthermore, the HULC A191G mutant and HULC mut mimics both abolished these interactions.

To facilitate the liver-enrichment of HULC mimics, three types of HULC mimics were applied: HULC mimics alone, peptides representing ApoE1 (Apo)-tagged HULC mimics, and N-Acetylgalactosamine (GalNAc)-tagged HULC mimics via intravenous (i.v.) or subcutaneous (SubQ) injection. GalNac-conjugated oligonucleotides have recently been suggested to assist with liver-targeted siRNA delivery (25). The 3′-triantennary GalNAc was conjugated to the HULC mimic.

A three-day treatment trial (see FIG. 5A in Li et al. 2021) was used, as it was considered that this would serve as the most convenient method to determine the efficacy of these mimics. Since PAH R408W-harboring patients respond to current BH₄ supplementation treatments poorly, female and male Pah^{R408W/R408W} mice were treated with the indicated mimics (data not shown; see FIGS. S19C and S19D in Li et al. 2021). GalNac-HULC administered via i.v. injection exhibited the highest efficacy in reducing excessive Phe concentrations in both female and male Pah^{R408W/R408W} mice.

The concentrations of biotin-labeled HULC or GalNAc-HULC mimics in the major organs were then determined, showing that a substantial portion of biotin-labeled GalNAc-tagged HULC mimics was detected in the liver between 3 and 72 hours after dosing, while the lungs and spleen exhibited no detectable accumulation of GalNAc-HULC (data not shown; see FIGS. S19E-519G in Li et al. 2021). Biotin was undetectable in the liver 12 hours post-injection (data not shown; see FIG. S19H in Li et al. 2021).

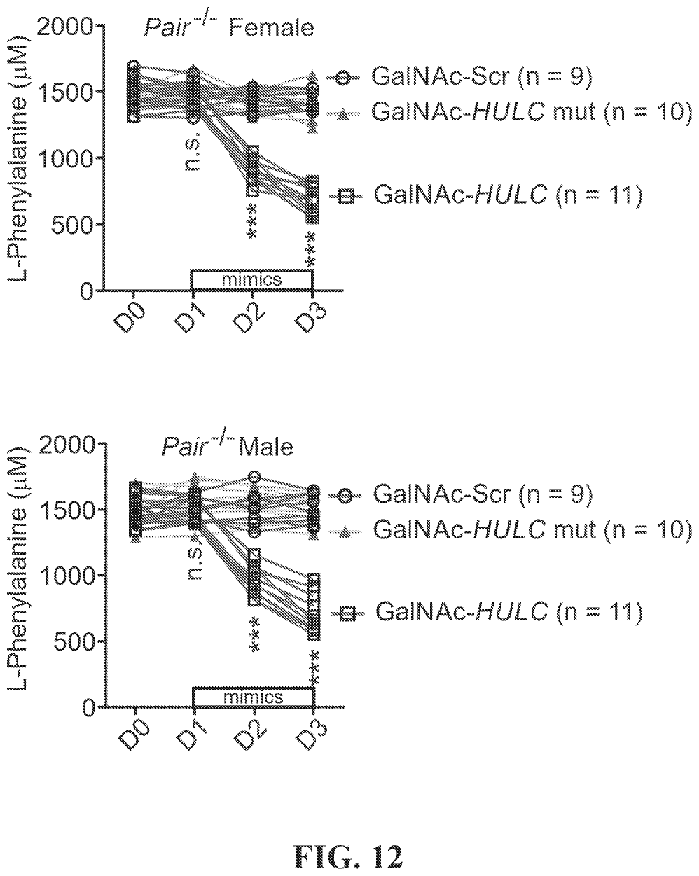

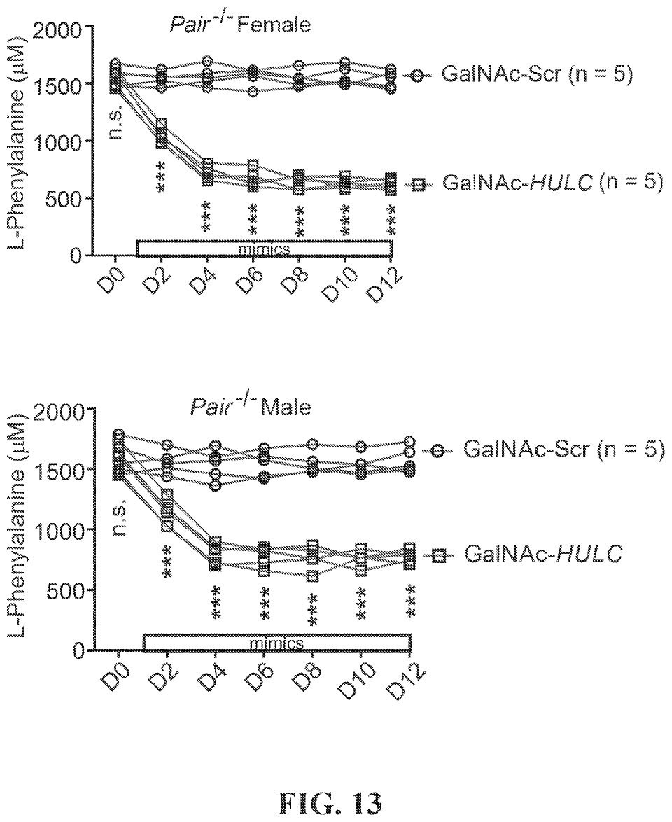

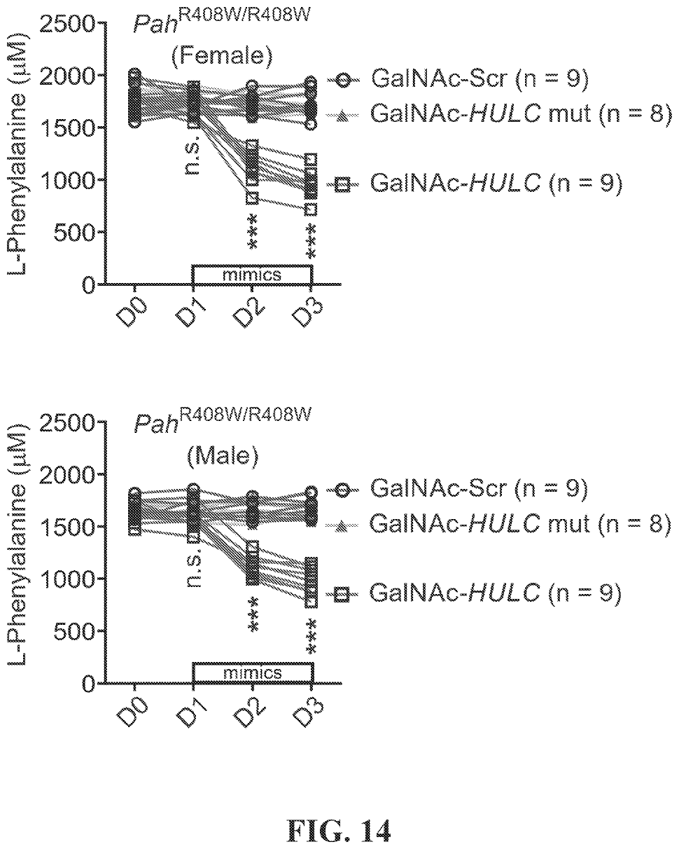

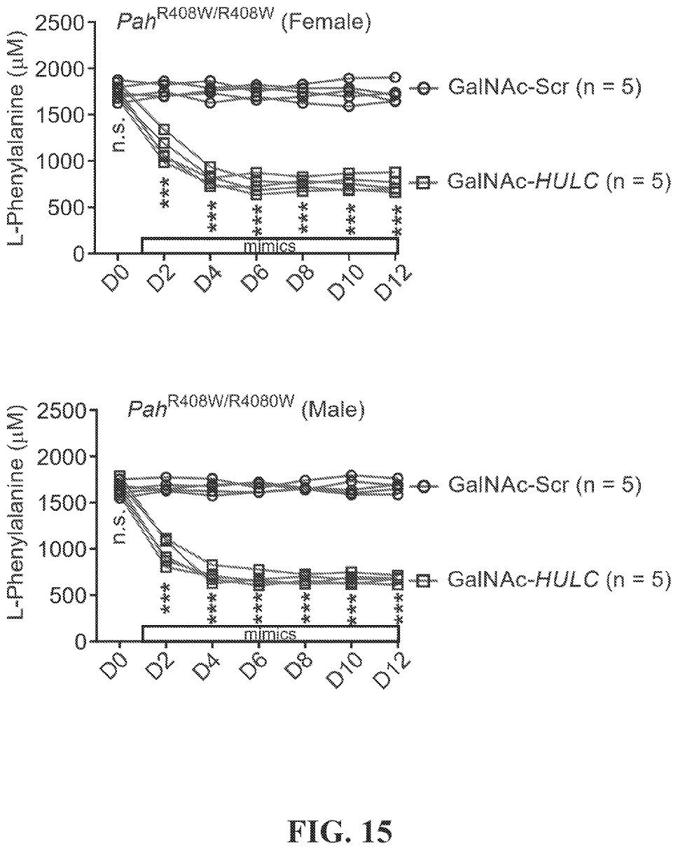

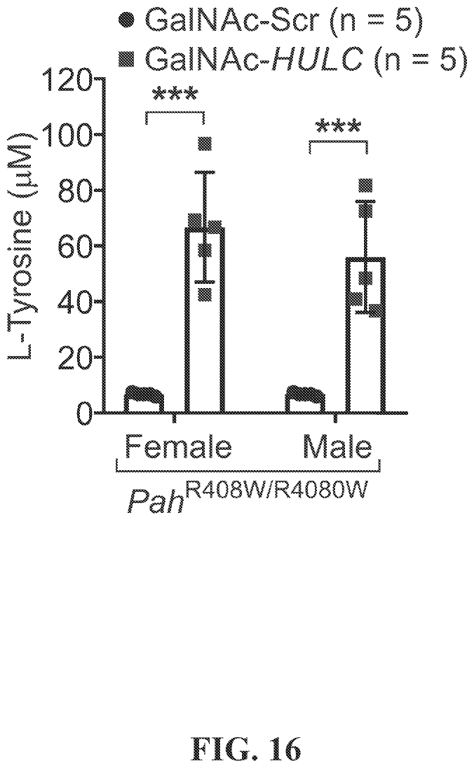

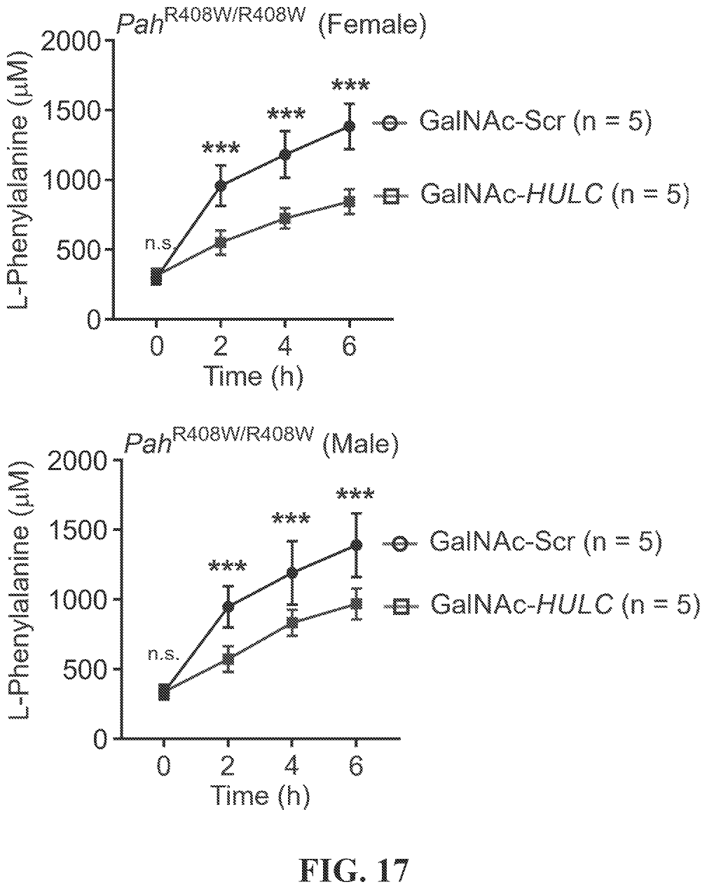

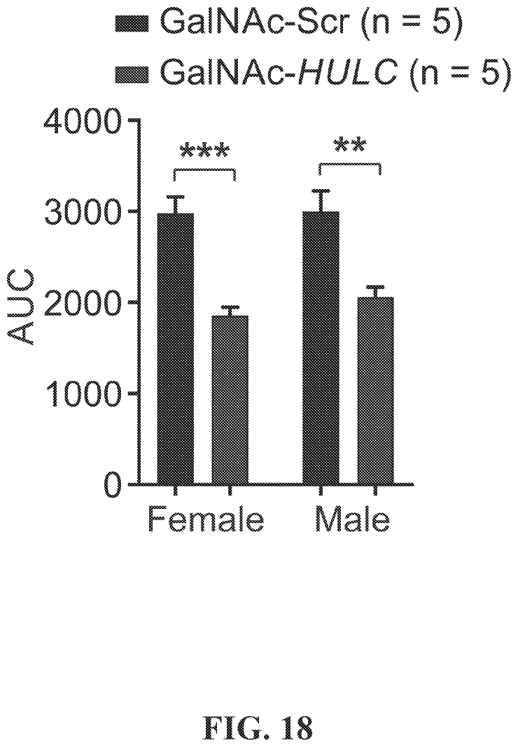

As a proof of concept, GalNAc-tagged Scr or HULC mimics were applied using short-term (3-day) and medium-term (12-day) treatment regimens (see FIG. 5A in Li et al. 2021). To rule out the indirect effects of the GalNAc tag, GalNAc-HULC mut mimics were included (FIG. 12). Both male and female Pair^{-/-} mice exhibited reduced serum Phe concentrations 24 hours after injection of GalNAc-HULC mimics, but not the Scr or HULC mut mimics (FIG. 12). During the medium-term treatment, administration of GalNAc-HULC mimics reduced blood Phe concentrations in Pair^{-/-} mice throughout the treatment term (FIG. 13). In the Pah^{R408W/R408W} mice, administration of GalNAc-HULC mimics similarly reduced blood Phe concentrations during the short-term and medium-term treatments (FIG. 14 and FIG. 15). The Pah^{R408W/R408W} mice subjected to GalNAc-HULC mimics showed increased blood Tyr concentrations compared to animals given scramble mimics (FIG. 16). To evaluate Phe clearance capacity, Pah^{R408W/R408W} mice were subjected to pre-treatment with Scr or HULC mimics followed by a Phe challenge (FIG. 17). The area under the curve (AUC) showed a reduction in Phe concentrations following GalNAc-HULC mimic treatment compared to GalNAc-Scr (FIG. 18).

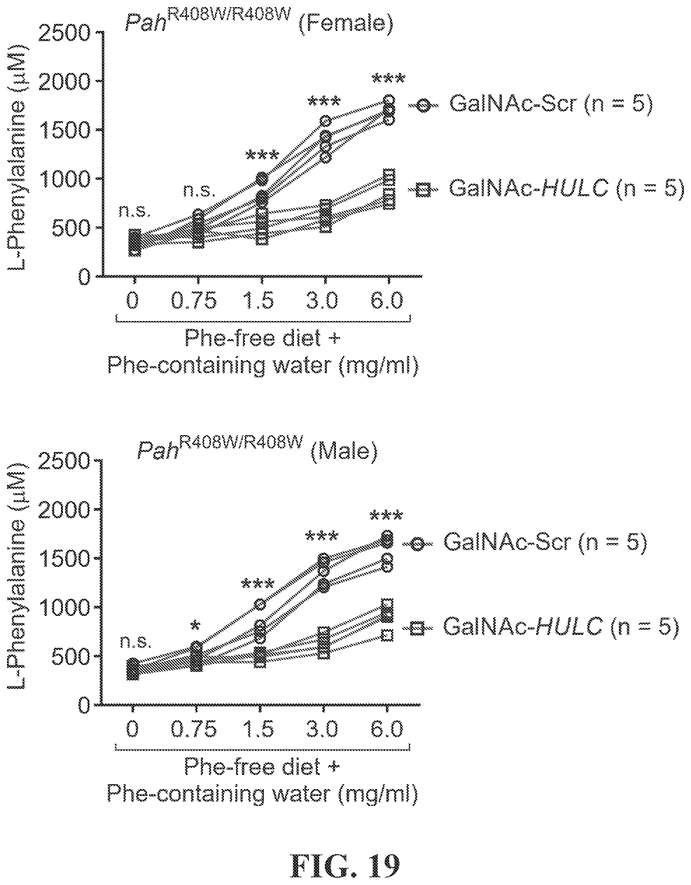

To determine whether the administration of GalNAc-HULC facilitates higher tolerance to dietary Phe intake in PKU animals, Pah^{R408W/R408W} were fed a Phe-free diet for three days followed by water containing increasing doses of Phe. Administration of GalNAc-HULC mimics allowed Pah^{R408W/R408W} animals to maintain relatively low blood Phe concentrations (<600 μM) upon Phe challenge up to 3.0 mg/ml (FIG. 19), suggesting that GalNAc-HULC mimics may also be able to improve tolerance to dietary Phe.

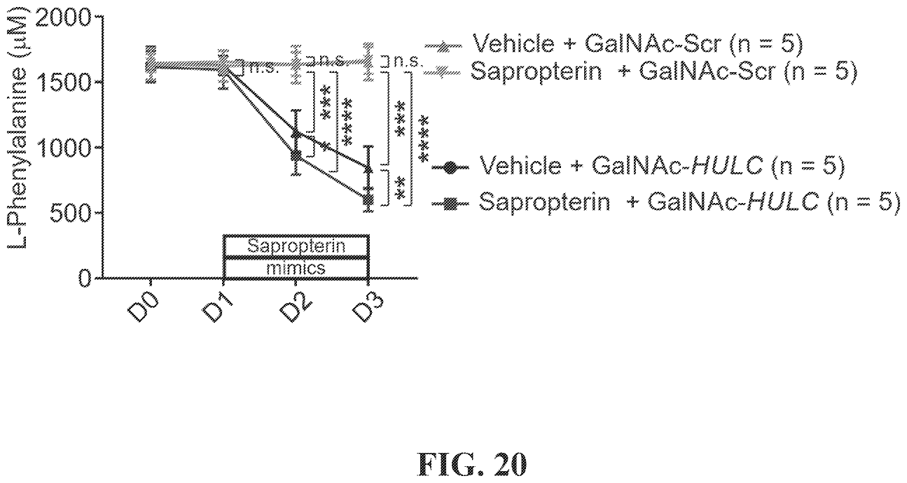

Sapropterin, a synthetic formulation of BH4, has been used as enzymatic enhancer for patients with HPA (26). Patients with PKU harboring R408W are resistant to sapropterin treatment (5). The efficacy of sapropterin in Pair^{-/-} and Pah^{R408W/R408W} mice was determined, revealing that supplementation of sapropterin showed no effect on the blood Phe of Pair^{-/-} and Pah^{R408W/R408W} animals (data not shown; see FIGS. S20A and S20B in Li et al. 2021). A combinational treatment of GalNAc-HULC and sapropterin showed a cooperative effect in reducing the blood Phe concentrations of Pah^{R408W/R408W} animals (FIG. 20). These findings suggested that a supply of GalNAc-HULC mimics may enhance the association between PAH and BH4, thereby improving the therapeutic effect of sapropterin. The HULC mimics showed no detectable effects on the body weight, liver function, and kidney function of Pair and Pah^{R408W/R408W} mice (data not shown; see FIGS. S20C-S20G in Li et al. 2021). Taken together, these data suggested that application of HULC mimics could enhance the enzymatic activities of certain mutated PAH proteins, offering a potential intervention for patients with PKU.

### Example 7. Additional Evaluation of HULC Mimics in PKU Model Mice

For toxicity evaluation, HULC mimics will be injected i.v in a single dose (0.15, 0.3, 0.6, 0.8, 1.2 mg/kg/i.v.; 10 female mice per group for six groups). Blood samples at 72 hour time points will also be analyzed for complete blood count (CBC) and blood chemistry. Complete blood cell counts (WBC, RBC, thrombocytes, lymphocytes, neutrophils, etc.), chemistry panels to assess for metabolic disorders, liver enzymes (AST, ALT, LDH) and renal markers (creatinine, BUN) will be analyzed to assess toxicity and immune response. Urinalysis for potential proteinuria, cast or cell will be determined for potential kidney damage. Erythrocyte sedimentation rate and C-reactive protein will be evaluated to determine the nonspecific elevation in acute-phase reactants for the potential immune response. Pathology will also be analyzed using H&E staining to look for evidence of preliminary toxicity to the brain, liver, kidneys, spleen, heart, lungs, intestines, and bone marrow.

In the previous Examples, short-term (3-day) and medium-term (12-day) efficacy was assessed in Pair^{-/-} and Pah^{R408W/R408W} mice by measuring serum phenylalanine levels before and after scheduled injections of mimics. LncRNA mimics were administered by intravenous injection at a dose of 1 mg/kg at 9:00 am on a set schedule. Blood samples were collected from tail veins before initial dosing and on subsequent scheduled days in the afternoon (at 2:00 pm). Increasing the dosage of HULC mimic may further reduce the blood phenylalanine concentration in PKU animals. lncRNA mimics will be applied daily at dosages of 1, 5, 10, and 20 mg/kg in Pair^{-/-} and Pah^{R408W/R408W} mice. Additionally, lncRNA mimics will be applied twice per day (9 am and 2 pm respectively) and the short-term (3-day) and medium-term (12-day) efficacy in Pair^{-/-} and Pah^{R408W/R408W} mice will be compared to determine the optimal condition of lncRNA mimics in reducing the blood phenylalanine concentration in mice modeling PKU disease. It is expected that increasing the dosage of lncRNAs mimics will further reduce the blood phenylalanine concentration and bring it close to normal level.

To further demonstrate the effect of lncRNA mimics in overcoming the neuronal damage of PKU disease, GalNAc-HULC mimics will be applied to both female/male Pair^{-/-} and PAH R408W mice using a long-term trial of 24 weeks. The mimics will be administered via subcutaneous injection every day at a dose of 1 mg/kg at 9:00 am on a set schedule. Body weight and coat color will be recorded every week and liver enzymes and kidney function markers will be assayed every two weeks. Blood concentrations of IL-1, IFN-γ, and TNF-α will be determined every four weeks to monitor immune response. The liver PAH enzymatic activities of these mice will be evaluated by phenylalanine clearance test and phenylalanine tolerance test. Brain weights and concentrations of monoaminergic neurotransmitters (catecholamine, norepinephrine, and serotonin) in brain will be determined. IHC detection of TH (tyrosine hydroxylase), NMDA (N-methyl-D-aspartate) receptor, and AMPA (α-amino-3-hydroxy-5-methyl-4-isoxazolepropionic acid) receptor will be determined as markers for neuronal damage recovery. General health condition will be monitored daily, and injection sites will be checked for signs of redness or edema. Whole blood count and histological analysis of major organs will be performed as primary indication of toxicity. It is expected that the long-term administration of GalNAc-HULC mimics could further reduce the blood phenylalanine concentration to normal level, leading to increased levels of monoaminergic neurotransmitters, expression of neuronal markers, and alleviated behavior defects.

The experiments described above used GalNAc-HULC mimics composed of 21 RNA nucleotides. Longer RNA mimics will also be tested. Further, Selective 2′ Hydroxyl Acylation analyzed by Primer Extension (SHAPE) assays and protein structural studies suggested that a 34 nt RNA fragment of HULC (SEQ ID NO:5) harbors stem-loop structures that associate with PAH through multiple hydrogen-bonds. As such, lncRNA mimics harboring 34 nt may improve the enzymatic activities of PAH mutants. Thus, lncRNA mimics harboring 34 nt wild type (SEQ ID NO:5) or mutant RNA sequence will be synthesized, with RNA nucleotide modifications as listed in Table 1, and tested. Pharmaceutical kinetics will be determined, as well as the in vivo toxicity and immunity of these lncRNA mimics. The efficacy of these lncRNA mimics in reducing blood Phe concentration in Pah^{R408W/R408W} mice will then be determined using short-term (3-day), medium-term (12-day), or long-term (24 weeks) treatment. It is expected that the Pah^{R408W/R408W} mice subjected to long lncRNA mimics will exhibit effectively reduced blood Phe concentration and improved neuronal function.

The data described above in the previous examples suggested that PAH R408W mice showed resistance to the treatment of Sapropterin. A phenylalanine (Phe)-low diet (glycomacropeptide diet) has been shown to reduce PKU patient plasma Phe concentrations and alleviate mental retardation (51). GalNAc-HULC or ApoE-HULC mimics will be applied (1 mg/kg, SubQ injection, every three days) to PAH R408W mice for a long-term trial of 24 weeks alone or in combination with each the following treatment strategies: a Phe-low diet, PAH mRNA (mRNA-3283), PAH protein, Pegvaliase, CDX-6114 (Codexia), SYNB-1618, Oxitriptan, Sepiapterin, or Sapropterin. BH₄ (Sapropterin dihydrochloride, MedChem Express, 20 μg/g bw b.i.d. by intraperitoneal injection for 3 days) or placebo (NaCl 0.9%, ascorbic acid 0.2%) will be used. The RNA mimics and combinatorial treatments will be administered at 9:00 am on a set schedule. Blood samples will be collected from the tail vein before initial dosing and on subsequent scheduled days in the afternoon (at 2:00 pm). Body weight and coat color will be recorded every week and liver enzymes and kidney function markers will be assayed every two weeks. Blood concentrations of IL-1, IFN-γ, and TNF-α will be determined every four weeks to monitor immune response. Both male and female animals aged 2-4 months will be used for the treatment experiment. General health condition will be monitored daily, and injection sites will be checked for signs of redness or edema. Whole blood count and histological analysis of major organs will be performed as primary indication of toxicity. At end of the experiment, animal blood chemistry, liver PAH enzymatic activities, expression of neuronal markers, and status of monoaminergic neurotransmitters will be determined.

For longer term studies, six groups of PAH R408 mice (PBS+Scr mimics, PBS+HULC mimics, Sapropterin+Scr mimics, Sapropterin+HULC mimics, Phe-low diet+Scr mimics, Phe-low diet+HULC mimics) will be compared. Identical experimental settings will be applied to GalNAc-HULC or ApoE-HULC mimics. The effect of two HULC mimic sequences will be determined in Year 2 and the effect of 2 other HULC mimic sequences will be determined in Year 3. The primary endpoint is the blood Phe concentration. Preliminary data indicated that the blood Phe concentrations of 6-8-week-old PAH R408 mice is, on average, 1600 μM. A t-test with two-sided 5% alpha, with 20 animals per group, will provide 99% power to observe a 50% reduction in the mean concentration of blood Phe.

Statistical analysis will be performed. For an effect size (ratio of fixed effect and residual standard deviation) of 0.65, the sample sizes described above will be sufficient to provide 80% power for a test at a significance level of 0.05. For analyzing associations among categorical variables, a Fisher-exact test will be used. For continuous and normally distributed variables, depending on the number of groups, a t-test or analysis of variance (ANOVA) (together with post-hoc tests) will be used to detect significant associations. Nonparametric tests (Mann-Whitney test, or Kruskal-Wallis, together with post-hoc tests, depending on the number of groups under consideration) will be used to assess statistically significant differences among groups for continuous variables that are not normally distributed. A Shapiro-Wilk test will be used to statistically test whether the data comes from a normal distribution. A p-value<0.05 will be considered statistically significant.

### Example 8. Materials and Methods

In vivo murine models and treatment procedures. All animal-based research was conducted according to the guidelines and requirements set forth by the Public Health Service (PHS) Policy on Humane Care and Use of Laboratory Animals, the U.S. Department of Health and Human Services Guide for the Care and Use of Laboratory Animals, and the Animal Welfare Act of 1966 as amended by the Institutional Animal Care and Use Committee (IACUC) of the University of Texas M.D. Anderson Cancer Center (MDACC). Pair^{-/-} mice were generated by CRISPR/cas9 Extreme Genome Editing System (EGE™, Biocytogen) to interrupt the splicing site after the first exon. Pah^{R408W/R408W} mice were also generated using Biocytogen's CRISPR/cas9 EGE system. The targeting vector and sgRNAs were injected to blastocysts and the high-percentage male chimeric mice were crossed with female mice to obtain transmitted founders. Single nucleotide polymorphism (SNP) genotyping of Pair^{-/-} and Pah^{R408W/R408W} mice was determined by TaqMan SNP genotyping technology (Applied Biosystems) from purified genomic DNA of mouse tails (Applied StemCell & Zymo Research). All mice were on a C57BL/6N genetic background. One founder Pair^{-/-} mouse and three PahR408W/R408W founder mice were achieved. All founder mice have been crossed with wild type C57BL/6N mice for at least three generations to dilute potential off-target effects. All founder animals passed the target sites: Chr6: 31,220,520G>A/31,220,522A>T (Pair^{-/-}) or chr10:87,581,870 (PAH R408W) to their offspring according to Mendel's laws. All the data reported are based on the observations of the progeny of all founder animals. Animals were housed in a controlled temperature room maintained under alternating 12 h light and dark cycles and, in between experiments, had free access to food and water. At least five mice were used in each group. Sample size was indicated in each figure.

Short-term (3-day) and medium-term (12-day) efficacy was assessed in Pair^{-/-} and Pah^{R408W/R408W} mice by measuring serum phenylalanine concentrations before and after scheduled injections of mimics. Both male and female animals at 2-4 months of age were used for the treatment experiment. lncRNA mimics were administered by intravenous injection at a dose of 1 mg/kg at 9:00 am on a set schedule. Blood samples were collected from tail veins before initial dosing and on subsequent scheduled days in the afternoon (at 2:00 pm). Serum phenylalanine concentrations were measured by EnzyChrom Phenylalanine Assay Kit (BioAssay Systems). For Phe-deficient diet, animals were fed with Phe-deficient diet (Envigo Teklad Diets, TD.01642), and supplied with Phe-containing water, with indicated concentrations. The effects of BH4 (sapropterin dihydrochloride, MedChemExpress, 20 μg/g body weight twice a day by intraperitoneal injection for 3 days) or placebo (NaCl 0.9%, ascorbic acid 0.2%) were assessed by blood phenylalanine concentrations. Animal weights were recorded to monitor animal well-being. General health condition, grooming, and behavior for all animals were monitored daily, and injection sites were checked for signs of redness or edema. Routine lab blood tests were measured using Cobas Integra 400 Plus (Roche Diagnostics). All mortalities were recorded and cause of death examined.

Phenylalanine clearance test. Phenylalanine clearance tests were performed in Pah^{R408W/R408W} mice at 8-12 weeks old, as previously described (31). Briefly, the mice received a Phe-free diet (Envigo Teklad Diets, TD.01642), GalNAc-Scr, or GalNAc-HULC mimics (1 mg/kg, i.v. daily) three days prior to Phe challenge. On the morning of hour 0, 5 mg/ml L-phenylalanine in Hank's buffered saline solution was administered via intraperitoneal injection (0.025 mg/g body weight). Serum Phe was measured prior to and 2, 4, and 6 h after injection and was plotted against time. The area under the curve (AUC) is plotted as inversely proportional to the rate of Phe clearance.

Phenylalanine tolerance test. Phe tolerance was determined using female and male Pah^{R408W/R408W} mice using age and gender matched littermates. Animals were fed a Phe-free diet (Envigo Teklad Diets, TD.01642) for three days, with administration of the indicated mimics (1 mg/kg, i.v. daily). Animals were then provided with Phe-containing water of increasing dosages (0.75, 1.5, 3.0 and 6.0 mg/ml every two days) for a period of 10 days. lncRNA mimics were administered by intravenous injection at a dose of 1 mg/kg at 9:00 am on a set schedule. Blood samples were collected from tail veins before initial dosing and on subsequent scheduled days in the afternoon (at 2:00 pm). Serum phenylalanine concentrations were measured as described above.

Synthesis of HULC mimics, GalNAc-HULC mimics, and Pharmacokinetics (PK) studies. The sequence of lncRNA mimics was synthesized by Bio-synthesis Inc. (sequences listed in Table 4). To characterize the pharmacokinetics of the HULC mimics and GalNAc-HULC, the biotinylated HULC or GalNAc-HULC mimics (10 mg/kg) were subcutaneously or intravenously injected into wild type C57BL/6 female mice. The liver, kidneys, lungs, and spleen of animals were collected 3, 12, 24, 48, or 72 h post-injection (n=5 animals per time point). Tissues were subjected to immunohistochemistry using streptavidin alkaline phosphatase conjugates and small RNA isolation as per the manufacturer's instructions using miRCURY RNA Isolation Kits (QIAGEN). Using a dot-blot, the biotin-labeled HULC mimics or GalNAc-HULC mimics were titrated using 2-fold dilutions ranging from 1 μg to 1 pg. The RNAs extracted from the liver, kidneys, lungs, and spleen were plotted at the indicated time points. The blotted biotin-labeled HULC mimics and GalNAc-HULC mimics were detected using streptavidin-HRP. By comparing the blot densities, the concentration of HULC mimics and GalNAc-HULC was calculated as ng/g tissue. Pharmacokinetic parameters were determined by nonlinear regression analysis.

SHAPE analysis by capillary electrophoresis. Selective 2′-hydroxyl acylation analyzed by primer extension (SHAPE) was performed as previously described with some modifications (32). Briefly, HULC RNA was synthesized with a MEGAscript T7 transcription kit (Invitrogen) and modified with N-methylisatoic anhydride (NMIA, 2.5 mM final, Invitrogen). Fluorescently-labeled cDNA products were reverse transcribed using 6-FAM and VIC labeled primers, priming the (+) and (−) reactions, respectively. Sequencing ladders were generated using the transcription template plasmid by performing primer extension using primers labeled with NED and PET in the presence of dideoxy NTPs (Thermo Sequenase Cycle Sequencing Kit, Applied Biosystems). The resulting cDNA products from both (+) and (−) reactions plus ddA and ddT sequencing reactions were combined and resolved in one multicolor run by capillary electrophoresis. GeneScan 600LIZ dye size standard was included for intercapillary alignment. Samples were analyzed using an Applied Biosystems 3730xl DNA analyzer. Primer sequences for capillary electrophoresis are listed in Table 4. For in vivo SHAPE analysis of HULC and Pair, hiPSC-derived hepatocytes or mouse hepatocytes FL83B overexpressing HULC or Pair were used. Cells were washed with cold PBS twice, scraped into PBS, pelleted and resuspended in PBS prior to the addition of 2-methylnicotinic acid imidazolide (NAI, sigma, 20 mM) or DMSO (33). Samples were placed at 37° C. for 20 min. Total RNA was purified using Trizol following the manufacturer's instruction. Fluorescently-labeled cDNA products were generated as described and were resolved with sequence ladders by capillary electrophoresis.

Cell lines and primary hepatocyte isolation. Mouse hepatocyte FL83B cells were purchased from American Type Culture Collection (ATCC, cat #ATCC® CRL-2390™) and maintained in F-12K Medium supplemented with 10% fetal bovine serum (FBS) at 37° C. in 5% CO2 (v/v). Immortalized human hepatocytes were obtained from ThermoFisher Scientific (cat #HMCPIS). Primary hepatocytes were isolated from Pair^{+/+} and Pair^{-/-} or PAH^{WT/WT} and Pah^{R408W/R408W} mice based on the methods of Li et al. with slight modifications (34). Perfusion (10 mM HEPES,0.5 mM EDTA) and digestion (0.5 mM CaCl2), 0.1% type I collagenase) solutions were freshly made in Ca²⁺- and Mg²⁺-free Hank's balanced salt solution (HBSS) and pre-warmed before isolation. Age-matched mice were anesthetized with ketamine/xylazine and a midline laparotomy was performed. The portal vein was visualized and a 23 gauge needle was inserted. The inferior vena cava was cut and the liver was perfused with perfusion solution at a flow rate of 5 ml/min for 10 min, followed by perfusion with digestion solution at a flow rate of 5 ml/min for 10 min. The gall bladder was removed and the liver was transferred into a 100-mm dish containing 10 ml of digestion buffer with antibiotics. The liver was gently minced with scissors and the cells were released. The cell suspension was filtered through a Falcon 100 pm Cell Strainer (Corning), and the cells were washed twice in Williams E medium (50 g for 5 min). Cell viability was determined by trypan blue exclusion and cells were seeded in collagen pre-coated plates and cultured with Williams E medium containing 1% Penicillin-Streptomycin at 37° C. under 5% CO2.

Human tissues, human induced pluripotent stem cells (hiPSC). and hiPSC-derived hepatocytes. De-identified fresh frozen human liver tissues were purchased from ProteoGenex Inc. The study protocol PA13-0330 was approved by the Institutional Review Board of MD Anderson Cancer Center, University of Texas. The commercially obtained human tissue samples are de-identified and informed consent has been waived. Clinical information is summarized in Table 2.

All hiPSC studies were approved by the HEIP Stem Cell committee of the University of Texas, MD Anderson Cancer Center, under protocol number SC00000021-RN00. The skin fibroblasts of healthy or PKU donors were obtained from Coriell Institute and reprogramed into hiPSC using Sendai virus as previously described (35). hiPSC characterization by karyotyping was performed at the Human Embryonic Stem Cell Core at Baylor College of Medicine. The hiPSC were further differentiated to hepatocytes using a StemXVivo Hepatocyte Differentiation Kit (R&D), in accordance with the manufacturer's instructions. Induced hepatocytes were further characterized by oil red o staining, AFP and Albumin immunofluorescence staining.

CRISPR/Cas9-mediated gene editing. HULC knockout cell lines were generated using the CRISPR/Cas9 genome editing system. Briefly, HULC or PAH specific guide RNA (gRNA) expression vectors were generated as described (36). LentiCas9-Blast (plasmid 52962; Addgene) and gRNA cloning vector lentiGuide-Puro (plasmid 52963; Addgene) were obtained from Addgene. The sequences of the sgRNAs are listed in Table 4. Hepatocytes were co-transfected with lentiCas9-Blast, gRNA1, and gRNA2 expression vectors. The transfected cells were selected using puromycin (0.5 μg/ml) and blasticidin (1 μg/ml). Isolated single colonies were subjected to detection of genomic deletions by PCR. Sequencing validation of the genomic deletions was performed after cloning the corresponding amplicon into pGEM-T-Easy (Promega).

Plasmid construction, recombinant protein expression, and transfection. The expression vector encoding full-length human PAH was purchased from the shRNA and ORF core of MD Anderson Cancer Center, and the coding regions were subcloned into Gateway™ pET-DEST40 vector for mammalian expression and Gateway™ pET-DEST42 vector for prokaryotic expression (Invitrogen). The Pair sequence was synthesized by GenScript and cloned into pGEM-3Z vector (Promega) for in vitro transcription. HULC sequence was PCR-amplified from human liver total RNA (Clontech) using LA Taq DNA polymerase (Clontech) and subcloned into pGEM-3Z vector (Promega). The mammalian expression vector for wild-type Pair and HULC was constructed by subcloning the gene sequences into pcDNA3.1 (+) backbone (Life technologies) and pMS2 vector. All mutants were generated by using QuikChange Lightning Site-Directed Mutagenesis Kit (Agilent Technologies).

Recombinant wild-type PAH and PAH mutants including F39L, A47V, F55L, I65S, P275L, P281L, 1283N, F299C, A300S, 1318T, R408W, Y414C, Y417H, and TH63-64PN were expressed in E. coli strain BL21-CodonPlus (DE3)-RIPL (Agilent Technologies) and purified using HisPur Cobalt Resin Kit (Thermo Scientific).

Plasmid transfections were performed using Lipofectamine3000 (Life Technologies) or electroporation using the 4D-Nucleofector™ System (Lonza) according to the manufacturer's instructions. Primary hepatocytes or hiPSC-derived hepatocytes were transfected using magnetic nanoparticles (Magnetofection, OZ Biosciences).

RNA pull-down and Mass spectrometry analysis. To identify Pair-binding proteins, Pair pulldowns were performed, as previously described (37). Briefly, biotin-labeled Pair RNAs were in vitro transcribed with Biotin RNA Labeling Mix (Roche) and MEGAscript Transcription Kit (Ambion) and further purified using RNA Clean & Concentrator-5 (Zymo Research). Mouse liver tissue lysates were freshly prepared using the RIPA buffer with an Anti-RNase, Protease/Phosphatase Inhibitor Cocktail supplemented in the lysis buffer. The eluted RNA-protein complexes were denatured, reduced, alkylated, and digested with immobilized trypsin (Promega) for mass spectrometry analysis at MD Anderson Cancer Center Proteomics Facility.

Limited proteolysis (LiP)-coupled liquid chromatography-mass spectrometry (LC-MS). LiP followed by LC-MS was modified based on LiP-SRM analysis (20). Briefly, bacterially expressed PAH (2 mg/ml), alone or in the presence of wild type HULC or the mutant transcript (2 mM) and/or phenylalanine (2 mM) or BH₄ (2 mM) were incubated in a buffer (20 mM HEPES, pH7.5, 150 mM KCl and 10 mM MgCl₂) and Protease K at room temperature for 5 min as limited proteolysis (20). The digestion was stopped by transferring the reaction mixture to a tube containing guanidine hydrochloride crystals to a final concentration of 7.4 M and boiling for 3 mins. The digestion mixtures were then subjected to complete Staphylococcal peptidase I and Arg-C proteinase digestion. The peptides were subjected to LC-MS analysis at the Proteomic and Metabolic core facility of MD Anderson Cancer Center. The number of peptides recovered from LiP-MS for each sample were normalized using only wild type PAH. Fold changes of peptides recovered from LiP-MS are shown.

Cross-linking immunoprecipitation (CLIP) and RIP assay. Liver tissues from Pair^{+/+} and Pair^{-/-} and human liver tissues were prepared and homogenized with a Dounce homogenizer, and the resulting suspension was crosslinked on ice with three irradiations of 254 nm UV-light at 400 mJ/cm² in a Stratagene crosslinker. CLIP was performed using a monoclonal PAH antibody (Santa Cruz), as described (38, 39). RNA-protein complexes of interest were then partially purified by immunoprecipitation, and non-covalently associated RNAs were removed by sodium dodecyl sulfate polyacrylamide gel electrophoresis (SDS-PAGE). These purified RNA-protein complexes were isolated and treated with proteinase K. The recovered RNAs were subjected to RNA linker ligation, RT-PCR amplification, cloning and Sanger sequencing. 32 individual clones for each sample were subjected to Sanger sequencing. RNA linkers for ligation: RL5: 5′-AGG GAG GAC GAU GCG G-3′ (SEQ ID NO:10); RL3: 5′-GUG UCA GUC ACU UCC AGC GG-3′ (SEQ ID NO:11). DNA primers for reverse transcription are DP5: 5-AGG GAG GAC GAT GCG G-3′ (SEQ ID NO:12); and DP3: 5′-CCG CTG GAA GTG ACT GAC AC-3′ (SEQ ID NO:13). RIP was performed using Magna RIP™ RNA-Binding Protein Immunoprecipitation Kit (Millipore). Primer sequences are listed in Table 4.

Dot blot assay. HULC binding selectivity to PAH, Phe, BH₄, or PIP₃ was assayed by a dot blot assay. Biotinylated PAH, Phe, BH₄ or PIP₃ were blotted on to SAM2 Biotin Capture Membrane (Promega). The membranes were then incubated with different binding probes such as digoxin (DIG)-tagged wild-type or mutant PAH G46S, F55L or P281L, DIG-tagged HULC and LINK-A, followed by detection using anti-digoxin-HRP.

In vitro translation and rapid amplification of cDNA ends (RACE) assay. In vitro translation was performed using TNT Quick Coupled Transcription/Translation Systems in the presence of Transcend Biotin-Lysyl-tRNA according to manufacturer's instructions (Promega). Reactants were visualized using SDS-PAGE followed by Streptavidin-HRP. 5′ and 3′ RACE were performed according to the manufacturer's protocol (Takara Bio) using an anti-sense primer (5′RACE) and a sense primer (3′ RACE) specific to the Mus Musculus RIKEN cDNA 2210408F21 gene. Mouse liver total RNA was used for cDNA synthesis. 5′/3′ RACE PCR products were ligated into a pRACE vector by In-Fusion cloning. Multiple isolated colonies were picked and sequenced by Sanger sequencing.

Measurement of RNA copy number and protein molecule number. The RNA copy number per cell measurements were performed as previously described (40). Briefly, total RNA was extracted and detected by qRT-PCR. A standard curve was generated by a serial dilution of in vitro transcribed Pair HULC RNA using the corresponding molecular weights, and the total RNA per cell was estimated to be 20 pg. The absolute molecule number of PAH and CD147 were determined using recombinant proteins with a serial-dilution as standard curves and cell lysates from 10,000 cells using PAH ELISA kit (Aviva Systems Biology) and CD147 ELISA kit (R&D systems).

Metabolomics Measurements. Livers were harvested from age-matched male adult mice and snap-frozen in liquid nitrogen. Blood was collected by cardiac puncture, processed for serum isolation 30 min after blood collection by centrifugation, and then snap-frozen in liquid nitrogen. All liver, serum samples and isolated Pair^{+/+} or Pair^{-/-} hepatocytes were stored at −80° C. until further analysis. Metabolomics analysis was carried out by Baylor College of Medicine Metabolomics Core Facility.

RNA in vitro transcription, LncRNA array and Northern blot. LncRNA sequences were in vitro transcribed using Biotin or Digoxin (DIG) RNA Labeling Mix (Roche). In vitro transcribed RNAs were purified using RNA Clean & Concentrator-5 (Zymo Research). Mouse lncRNA array hybridization and data analysis were performed as previously described (41). The expression of Pair, β-actin, and HULC in different types of tissues was analyzed by NorthemMax Kit (Invitrogen) using biotin-labeled LNA Probes (Exiqon). Probe sequences are provided in Table 4.

Immunohistochemistry, image analysis, and quantification. Mice were anesthetized and perfused intracardially with fresh 4% paraformaldehyde/PBS buffer. Brains were collected and post-fixed for 24 h at 4° C. Paraffin embedded tissues were deparaffinized and rehydrated, followed by antigen retrieval. After primary and secondary antibody incubation, the slide was dehydrated and stabilized with a mounting medium. All immunostained slides were scanned on the APERIO Scan Scope XT (Leica Biosystems) for quantification by digital image analysis. The quantification of IHC staining density was performed by Image-pro plus 6.0 software (Media Cybernetics) and calculated based on the average staining intensity and the percentage of positively stained cells.

RNA Fluorescence In Situ Hybridization. RNA FISH was performed using LNA FISH technology according to manufacturer's instructions (Exiqon) with minor modifications. Briefly, cells with the indicated treatments were fixed in 4% formaldehyde/5% acetic acid for 15 min followed by washes with PBS. The fixed cells were further treated with pepsin (1% in 10 mM HCl) and subsequent dehydration through 70%-, 90%- and 100% ethanol. The air-dried cells were subjected to incubation with a 40 nM LNA FISH probe in hybridization buffer (100 mg/ml dextran sulfate, 10% formamide in 2×SSC) at 80° C. for 2 min. The hybridization was performed at 55° C. for 2 h, and the slide was washed with 0.1×SSC at 65° C. followed by dehydration through 70%-, 90%-, and 100% ethanol. The air-dried slide was mounted with Prolong Gold Antifade Reagent with DAPI for detection. An LNA FISH probe targeting HULC and Pair was designed using Exiqon's online design software, and the control probe targeting PAH was purchased from Exiqon (sequences are listed in Table 4). The images were visualized with a Zeiss Axioskop2 plus Microscope. FUJI Coloc 2 analysis was used to determine the Pearson's R value (above threshold).

RNA Electrophoretic Mobility Shift Assay (EMSA). The gel mobility shift assay was performed as previously described (42). Briefly, 500 ng recombinant His-tagged wild-type PAH or mutants were incubated with 0.035 μmol labeled probe in a RNA-protein binding buffer [(50 mM Tris-HCl 7.9, 10% glycerol, 100 mM KCl, 5 mM MgCl₂, 10 mM (3-ME, 0.1% NP40] for 30 min at 30° C. For cold RNA competition, 0.035 μmol radiolabeled RNA probes were first mixed with 7 μmol cold RNA competitors and then subjected to the gel shift assay.

In vitro phenylalanine hydroxylase assay. The assays were conducted as previously described (43). Briefly, reactions were in a 0.02 M potassium phosphate buffer, pH 6.8, 25° C., containing 0.1 M KCl and 10-15 pg catalase/ml. To reduce phenylalanine hydroxylase, wild-type enzyme or indicated mutants was incubated with 1.52.5 molar equivalents of 6 M BH4 for 2 min (25° C.). The activated enzymes were further incubated with the Newborn's Heel Blood Spot provided with the Phenylalanine (PKU) neonatal assay kit (DRG International, Inc.) in the presence or absence of the indicated RNA. The commercially obtained human tissue samples are de-identified and informed consent has been waived. After incubation, the concentrations of phenylalanine and tyrosine were measured by ELISA (enzyme-linked immunosorbent assay) according to vendor's instructions (DRG International, Inc. and BioVision, respectively).

Cellular amino acid concentrations (phenylalanine, tyrosine, tryptophan) and BH4 measurements. Cellular and PAH associated amino acid concentrations were determined using phenylalanine (DRG International, Inc.), tyrosine (BioVision), and tryptophan (Rocky Mountain Diagnostics) ELISA kits. Cellular and PAH-associated BH₄ concentrations were determined using a Tetrahydrobiopterin ELISA Kit according to vendor's instructions (Aviva Systems Biology).

PAH enzymatic activity measurements. Wild-type and mutated PAH enzyme activity were assessed by detection of an increase in fluorimetric intensity of L-Tyr with reaction mixtures containing 1 mg/ml catalase, 10 μM ferrous ammonium sulfate, and the indicated concentrations of L-Phe and 75 μM BH₄. Fluorescence intensity was converted to enzyme activity units (nmol Tyr/min*mg protein) using the standard curve obtained by L-Tyr titration (44).

Measurement of Tyrosine formation in liver slices and hepatocytes. Tyrosine formation in Pair^{+/-} and Pair^{-/-} liver slices was determined as previously reported (45). Briefly, liver slices from the desired animals were incubated in a 25 ml conical flask in a total volume of 2 ml containing 0.1 M potassium phosphate buffer, pH 6.8, and 1 μCi of L-[¹⁴C]phenylalanine plus added cold L-Phenylalanine, as indicated. After incubation, the reactions were stopped by the addition of 0.3 ml of 3 M HClO₄. The medium and the slices were homogenized and centrifuged to remove debris. For measurements of PAH enzyme activity in hepatocytes, cell pellets were disrupted in a buffer containing 0.2 M KCl, 0.05 M K₂HPO₄ (pH 7.0), 1 M DTT, 0.7% Triton X100, and protease inhibitors (PMSF, leupeptin, aprotenin, and lysozyme) by vortexing repeatedly for 40 min on ice, then centrifuging at 4° C. for 25 min at 20,800 g. PAH activity was assayed in vitro by measuring the production of ¹⁴C-tyrosine from ¹⁴C-phenylalanine in the presence of a crude cellular extract and the natural cofactor BH₄. The assay was performed with a 50 ml volume containing 0.2 M KCl, 0.04 M K₂HPO₄ (pH 7.0), 0.2 mM phenylalanine, 0.1 mM Iron (II) ammonium sulphate, 0.1 mCi [¹⁴C(U)]-phenylalanine (PerkinElmer), 10 mg catalase (Sigma), and 450 mg crude extract. The reaction was started with the addition of BH4 (Sigma-Aldrich) and DTT (final concentrations 100 mM and 4 mM, respectively). After incubation for 60 min at room temperature, the phenylalanine hydroxylation reaction was stopped by adding excess unlabeled phenylalanine and tyrosine. Proteins were precipitated with TCA and pelleted by centrifugation for 5 min. ¹⁴C-phenylalanine and ¹⁴C-tyrosine were separated on a TLC-plate precoated with cellulose (Cat #105730, Millipore) in a solution of chloroform:methanol:ammonia (55:35:10) and visualized on a phosphorimager screen. Quantification was done using Image J.

Chemical pull down. The biotin-linker-phenylalanine (Bio-Phe), biotin-linker-BH₄ (Bio-BH₄), and control compound biotin-linker (Bio-Linker) were custom synthesized by KareBay Biochem Inc. The cell lysates from Pair^{+/+} and Pair^{-/-} hepatocytes were freshly prepared using the ProteoPrep Zwitterionic Cell Lysis Kit, Mass Spec Grade (Protea) with Anti-RNase, Protease/Phosphatase Inhibitor Cocktail supplemented in the lysis buffer. The Bio-Linker, Bio-Phe, and Bio-BH₄ were coupled with streptavidin-coupled Dynabeads (Thermo Fisher Scientific) according to the manufacturer's instructions. The biotinylated compound-protein complexes were washed sequentially with NT2 buffer (50 mM Tris-HCl pH 7.4, 150 mM NaCl, 1 mM MgCl₂, 0.05% NP-40, twice), NT2-high-salt buffer containing 500 mM NaCl (twice), and PBS (once) for 5 min at 4° C. and eluted by NuPAGE LDS Sample Buffer (Thermal Fisher Scientific) boiled for 10 min. The eluted protein was analyzed by western blot.

RNA isolation, qRT-PCR, Cell lysis, and immunoblotting. Total RNA was isolated from cells using RNeasy Mini Kit (QIAGEN) following the manufacturer's protocol. First-strand cDNA synthesis from total RNA was carried out using iScript Reverse Transcription Supermix for RT-qPCR (Bio-Rad). The primer sequences are listed in Table 4. To determine the expression status of 2210408F21Rik isoforms, isoform-specific primers were used for NR 040257, NR 040259, NR_040260, NR 040261, and NR_040262. Primers targeting NR_040258 recognize both NR_040258 and NR_040257. The expression status of each isoform was presented as a percentage of GAPDH. The percentage of NR 040258 was determined by subtracting the percentage of NR_040257. Cells were homogenized in a 1×RIPA buffer (EMD Millipore) supplemented with Protease/Phosphatase Inhibitor Cocktail (Pierce, Thermo Scientific), panobinostat (Selleck chemicals), and methylstat (Sigma-Aldrich). Lysates were cleared by centrifugation at 13,000 rpm for 15 min at 4° C. Supernatants were subjected to immunoblotting. The antibodies used in the study are listed in Table 3.

TABLE 3
Antibodies used in the experiments described herein.

| Primary | | Catalogue | | | |
|---|---|---|---|---|---|
| Antibodies | Company | Number | Clone | Dilution | Application |
| PAH | Santa Cruz | sc-271258 | H2 | IHC, 1:200; | IHC, IF, |
| | Biotechnology | | | IF, 1:200; | IB, IP |
| | | | | IB, 1:2000; | |
| GAPDH | Santa Cruz | sc-32233 | 6C5 | 1:3000 | IB |
| | Biotechnology | | | | |
| GST-Tag | Cell Signaling | 2624 | 26H1 | 1:2000 | IB, IP |
| | Technology | | | | |
| His-Tag | Cell Signaling | 12698 | D3I1O | 1:2000 | IB, IP |
| | Technology | | | | |
| Tyrosine | Santa Cruz | sc-25269 | F11 | 1:200 | IHC |
| hydroxylase | Biotechnology | | | | |
| FLAG-Tag | Sigma-Aldrich | F1804 | M2 | 1:3000 | IB |
| IgG | Santa Cruz | sc-515946 | D1 | 1:200 | IB, IP |
| | Biotechnology | | | | |
| PODXL | LSBio | LS-C295987 | Polyclonal | 1:200 | IB |
| MKLN1 | LSBio | LS-C747296 | Polyclonal | 1:500 | IB |
| KLF14 | LSBio | LS-C778933 | Polyclonal | 1:500 | IB |
| PCBD1 | Abclonal | A6392 | Polyclonal | 1:500 | IB |
| PTS | Abclonal | A6306 | Polyclonal | 1:500 | IB |
| QDPR | Abclonal | A5733 | Polyclonal | 1:500 | IB |
| AFP | Santa Cruz | sc-8399 | C3 | 1:100 | IF |
| | Biotechnology | | | | |
| Albumin | R&D | MAB1455 | 188835 | 1:100 | IF |
| HuR | Santa Cruz | sc-5621 | 3A2 | 1:200 | IB |
| | Biotechnology | | | | |
| snRNP70 | Sigma-Aldrich | 03-103 | Polyclonal | 1:200 | RIP |

| | | Catalogue | | |
|---|---|---|---|---|
| Secondary Antibodies | Company | Number | Dilution | Application |
| Goat anti-Mouse IgG1 | ThermoFisher | A-21121 | 1:300 | IF |
| Alexa Fluor 488 | | | | |
| Goat anti-Mouse IgG1 | ThermoFisher | A-21125 | 1:300 | IF |
| Alexa Fluor 594 | | | | |
| Goat anti-Rabbit IgG | ThermoFisher | A-11034 | 1:300 | IF |
| Alexa Fluor 488 | | | | |
| Goat anti-Rabbit IgG | ThermoFisher | A32740 | 1:300 | IF |
| Alexa Fluor 594 | | | | |
| Goat anti Rabbit IgG | Bio-Rad | STAR208P | 1:5000 | IB |
| (H/L): HRP | | | | |
| Goat anti Mouse IgG | Bio-Rad | STAR207P | 1:5000 | IB |
| (H/L): HRP | | | | |
| Streptavidin-HRP | Cell Signaling | 3999S | 1:2000 | IB |
| | Technology | | | |

Determination of K_{d} value using alpha assay. Alpha binding assay was used to determine K_{d} for HULC mimics, Bio-Phe, Bio-BH₄, and His-PAH WT, as well as the mutants' interactions, performed as previously described (19). The K_{d} was determined by a competition experiment in which unlabeled competitors as indicated in the figure legend were titrated (2-fold dilution) from 10 μM to 0.1 nM. Streptavidin donor beads and anti-His6 AlphaLISA acceptor beads were used in these assays (PerkinElmer). The plate was read on an EnSpire Multimode Plate Reader (PerkinElmer). The competitive inhibition curves were calculated based on alpha signal readings by fitting to a “log (inhibitor) vs. response-variable slope (four parameters)” model (GraphPad Prism 8 software).

To determine the K_{d} value of the PAH-Phe or PAH-BH₄ interaction with lncRNA mimics, Alpha assays were performed as described above. Unlabeled Phe or BH₄ was titrated (2-fold dilution) from 10 μM to 0.1 nM. The K_{d} values between PAH-Phe or PAH-BH₄ were determined using GraphPad Prism 8 software.

Model PAH 3D Structures. As the goal was to elucidate the effect of HULC binding on the phenylalanine-bound (open) state of PAH, the activated PAH structure with loop optimization was built using Modeller v9.24 (46). Since no full-length human activated PAH structure was available, the 3D models were constructed based on two crystal structures: one using full-length PAH (PDB: 6HYC) and the other using the phenylalanine-bound regulatory domain of PAH (PDB: 5FII). All default parameters were used unless otherwise stated. The derived structure was further prepared and refined using MOE v2019.01 (47).

Prediction of PAH-RNA Interactions. To model the PAH-RNA interactions, the 3D structure of the stem loops of HULC/Pair was initially built using MC-fold/MC-Sym. These RNA structures and the above derived PAH protein model were used as the input ligand and receptor, respectively, for the docking studies. Then, protein-RNA docking was performed using multiple web servers including 3dRPC (48), HDOCK (49), and NPDock (50). Energy minimizations of the docked complex structures were then conducted in MOE. For each docking experiment, the top 100 poses with the best scores were selected and examined for interactions using Pymol v2.4, along with the final image/figure generation.

Statistics and reproducibility. The experiment was set up to use 3-8 samples/repeats per experiment/group/condition to detect a 2-fold difference with a power of 80% and at the significance level of 0.05 by a two-sided test for significant studies. No samples were excluded from the data analysis. Each of these experiments was independently repeated 3-8 times. Results are reported as mean±standard error of the mean (S.E.M.) or standard deviation (SD) of at least three independent experiments, as indicated by figure legends. Each exact n value is indicated in the corresponding figure legend. Statistical analysis was performed using GraphPad Prism 8 software. Comparisons were analyzed by Student's t-test (paired or unpaired), one-way ANOVA test, two-way ANOVA test (n.s., p>0.05, *p<0.05, **p<0.01, and ***p<0.001), as indicated in individual figures. Kaplan-Meier survival curves were compared using the log rank test.

TABLE 4
Selected sequences.

| | | SEQ |
|---|---|---|
| lncRNA | | ID |
| sequences | Sequence (5′→3′) | NO |
| Pair | GUGUCAUGCAAGAAGGAGGCGGCGGCGGCGGCCGGAGCUGGAGGGGGAG | 1 |
| (NR_040259.1) | GAGGGGAGGAACCUGAUCCUCGGUAGCGAAGGGCUGGAGGCAAACACCG | |
| | AGCUCCCCCUGAGCCGCCUGGGAGCAGGGAGGGUUCUGCUACCCUGAGG | |
| | GGCCCUCCCCGCGGGGCAGAGGGGUUUCCUCAGCAGUGCCCCGGAGCAG | |
| | GGACCGCGCCAGCCAGCAGAAACAGCUAGUUGUUGUCUGCAGUGAAGAA | |
| | UAAUUGGAAGCAUGGUGCCUGUUUCUUUUCCUGUGGGAGGAAGUCACA | |
| | GCAUCUACUUCUGAAUCUGUGCUUGCCUUGAGGAAAAACGGCAUGGCAC | |
| | AGGAAUGUGGACGCUUUCAACCAGAGGAUUCUGUACGACGGUGUCGGCA | |
| | CACAAGGAGUGUUCCACUGACUGACCUCUUUUUGUUUUCAAAUGAAGAG | |
| | GAGCCAAGAAUAGGACAGCUUGGGAAGGUGAGCUGCGUAUAGCUCGUCA | |
| | GGCUGAGCUCAUCCUCGUCUACGGCCAAACGUUGGUUUCUCAUUUCUAG | |
| | AAGAUGAAAGAUGUGAUGACUCAGGAAAAACUUGCUGCUCGAGAUGUG | |
| | AGUCUUCAGAUGUUCCUGCCGCCAUAUCUGACAGCUGCUGCCAAGAUUC | |
| | CCUGGUGGGAUGGACUCUUAUACCUCAACAACCAGAAGCCCAAGUAAAC | |
| | UCUUCUAUAAAUUGAAUGACUCAUGCUUAAAAAAAAAAACAAAAAACA | |
| | AA | |
| HULC | AUGGGGGUGGAACUCAUGAUGGAAUUGGAGCCUUUACAAGGGAAUGAA | 2 |
| (NR_004855.2) | GAGACAAGAGCUCUCUUUAUGCCACGUGAGGAUACAGCAAGGCCCCAAU | |
| | CUGCAAGCCAGGAAGAGUCGUCACGAGAACCAGACCAUGCAGGAACUCU | |
| | GAUCGUGGACAUUUCAACCUCCAGAACUGUGAUCCAAAAUGCAUAUGUA | |
| | UCUUUGGAAGAAACUCUGAAGUAAAGGCCGGAAUAUUCUUUGUUUAAA | |
| | ACAUUAAAAACAAAACAGACCAAAGCAUCAAGCAAGAAGUUUCCUGGCA | |
| | AUAAACUAAGCACAGCAUUAUUUUUUAAGGAACACAAAUUAAGUGUUC | |
| | AACCUGUGGCAAAUUUGUACUUUCUCCCUGAAUUAUGUUGUUAUCAAAG | |
| | AAAAAAAUUGGGAAGCAUGGCAAAAUAUCAUCAAAACUGAAACUAGAA | |
| | UUAAACAAAACUAAAUUAAAAUGAAAUAAAAUGAUGUCCAUUCUUAAA | |
| | AAAAAAAAAAAAAAA | |
| Pair 460-496 | UUGGGAAGGUGAGCUGCGUAUAGCUCGUCAGGCUGAG | 3 |
| Pair 470-488 | GAGCUGCGUAUAGCUCGUC | 4 |
| HULC 183-216 | AAAUGCAUAUGUAUCUUUGGAAGAAACUCUGAAG | 5 |
| HULC 183-200 | AAAUGCAUAUGUAUCUUU | 6 |
| HULC 181-201 | CAAAAUGCAUAUGUAUCUUUG | 7 |
| HULC 181-201 | CAAAAUGCAUGUGUAUCUUUG | 8 |
| mutant | | |
| Peptide tag | Sequence (N→C) | |
| ApoE peptide | LRKLRKRLLLRKLRKRLL | 9 |
| CLIP and RIP | | |
| sequences | Sequence (5′→3′) | |
| RNA linker | AGGGAGGACGAUGCGG | 10 |
| RL5 | | |
| RNA linker | GUGUCAGUCACUUCCAGCGG | 11 |
| RL3 | | |
| DNA primer | AGGGAGGACGATGCGG | 12 |
| DP5 | | |
| DNA primer | CCGCTGGAAGTGACTGACAC | 13 |
| DP3 | | |
| Northern blot | | |
| probes | Sequence (5′→3′) | |
| Pair | AGGCTATCTGGACAAATGCACT | 14 |
| HULC | TGCCACAGGTTGAACACTTAAT | 15 |
| b-Actin | CTCATTGTAGAAGGTGTGGTGCCA | 16 |
| RNA FISH | | |
| probe | Sequence (5′→3′) | |
| Scramble | /56-FAM/GTGTAACACGTCTATACGCCCA | 17 |
| b-Actin | /56-FAM/CTCATTGTAGAAGGTGTGGTGCCA | 18 |
| Pair | /56-FAM/AGAGGTCAGTCAGTGGAACACT | 19 |
| Tug1 | /56-FAM/ATAGCTGGCAGTGTAGCTTAT | 20 |
| HULC | /56-FAM/TGCCACAGGTTGAACACTTAAT | 21 |
| TUG1 | /56-FAM/AGTGAGCTGAATAGTCATAAT | 22 |
| RT-qPCR | | |
| primers | Sequence (5′→3′) | |
| NR_040259 | TGGACGCTTTCAACCAGAGGAT | 23 |
| (Pair)-F | | |
| NR_040259 | CTATACGCAGCTCACCTTCCCA | 24 |
| (Pair)-R | | |
| HULC-F | TCGTCACGAGAACCAGACCATG | 25 |
| HULC-R | GCTGTGCTTAGTTTATTGCCAGG | 26 |
| U1snRNA-F | TTTTCCCAGGGCGAGGCTTA | 27 |
| U1snRNA-R | CCCCACTACCACAAATTATGCAG | 28 |
| mLinc-Pint-F | AGAGAGCAAAGCGGTGTAGTGT | 29 |
| mLinc-Pint-R | ATCAGCAAGGCAGAGAGGTGG | 30 |
| mLinc-YY1-F | AGTTACAGGGAAGTTTGGGCTAC | 31 |
| mLinc-YY1-R | AGGCAAAGGACGGCTGTGAG | 32 |
| hPAH-F | GCCACTGTCCATGAGCTTTCAC | 33 |
| hPAH-R | AGCATCCAGTTCCGCTCCATAG | 34 |
| Cd147-F | TGGATGAGAGGTGGCAAGGTAC | 35 |
| Cd147-R | GCTCAGGAAGGAAGATGCAGGA | 36 |
| mPts-F | CACCGGCTGCACAGATTGATC | 37 |
| mPts-R | GCACATCCAGGTCCAGGTTCTT | 38 |
| mGCH1-F | TGGTCTCAGTAAACTTGCCAGGA | 39 |
| mGCH1-R | CAATCACTACTCCAACGCCAGC | 40 |
| mQDPR-F | GCAATTCTCTGTGTGGCTGGAG | 41 |
| mQDPR-R | GAGTCCCATCCAAGGCAGCTTT | 42 |
| mPCBD1-F | GGTGGAATGAAGTAGAAGGCCG | 43 |
| mPCBD1-R | TGGGTGCTCAAGGTGATATGGA | 44 |
| mSPR-F | CTCATCAACAACGCAGCCACTC | 45 |
| mSPR-R | CCGGAAGTCAAACAGAGCATGG | 46 |
| GAPDH-F | AGGTCGGTGTGAACGGATTTG | 47 |
| GAPDH-R | TGTAGACCATGTAGTTGAGGTCA | 48 |
| HULCseq-F | GCCAGAGGATAAAGCAAGCTG | 49 |
| HULCseq-R | TCACCTTCCAAATTACCTCCTTT | 50 |
| HULCseq F1 | TTCACTATCTGGTGCTGAAGC | 51 |
| mKlf14-F | TACCGAAGGAGGCAGATTACGC | 52 |
| mKlf14-R | GTCGAGCCAATCACAGGAGAAG | 53 |
| mMkln1-F | GTGTGAAGGAGAACCAGTGGAC | 54 |
| mMkln1-R | CACAGAGGAATCCAAGTAACGGC | 55 |
| mPodx1-F | ATAACCAGGCGGTGGCAGTGAA | 56 |
| mPodx1-R | CCAGCTTCATGTCACTGACTCC | 57 |
| NR040257-F | AGAAAGGAGGGTGATGCTGTGT | 58 |
| NR040257-R | CCCTGTGAAGGTTGAAATGGCT | 59 |
| NR040258-F | CTTTTCCTGTGGGAGGAAGTCA | 60 |
| NR040258-R | AGGATGAGCTTCCCAAGCTGTC | 61 |
| NR040260-F | CAGAGGGTGGGAGGAAGTCAC | 62 |
| NR040260-R | ATCCTCTGGTTGAAAGCGTCCA | 63 |
| NR040261-F | GGCACACACTCATCCTCGTCTA | 64 |
| NR040261-R | CTTGGGCTTCTGGTTGTTGAGG | 65 |
| NR040262-F | GCCTGTTTCTTTTCCTAGGAGCC | 66 |
| NR040262-R | CTCAATGCCCACCACAGTTACT | 67 |
| EMSA probes | Sequence (5′→3′) | |
| HULC180-202 | CCAAAAUGCAUAUGUAUCUUUGG | 68 |
| WT | | |
| HULC180-202 | CCAAAAUGCAUGUGUAUCUUUGG | 69 |
| mutant | | |
| Pair 467-488 | GGUGAGCUGCGUAUAGCUCGUC | 70 |
| WT | | |
| Pair 467-488 | GGUGAGCUGCGUGUAGCUCGUC | 71 |
| mutant | | |
| lncRNA | | |
| mimics | Sequence (5′→3′) | |
| Scramble | 5′-[2′-F-U][2′-F-U][2′-F-C][2′-F-U][2′-F-C][2′-F-C][2′-F-G][2′-F-A] | 72 |
| | [2′-F-A][2′-F-C][2′-F-G][2′-F-U][2′-F-G][2′-F-U][2′-F-C][2′-F-A] | |
| | [2′-F-C][2′-F-G][2′-F-U][2′-F-U][2′-F-U]-3′ | |
| HULC | 5′-[2′-F-C][2′-F-A][2′-F-A][2′-F-A][2′-F-A][2′-F-U][2′-F-G][2′-F-C] | 73 |
| | [2′-F-A][2′-F-U][2′-F-A][2′-F-U][2′-F-G][2′-F-U][2′-F-A][2′-F-U] | |
| | [2′-F-C][2′-F-U][2′-F-U][2′-F-U][2′-F-G]-3 | |
| HULC mutant | 5′-[2′-F-C][2′-F-A][2′-F-A][2′-F-A][2′-F-A][2′-F-U][2′-F-G][2′-F-C] | 74 |
| | [2′-F-A][2′-F-U][2′-F-G][2′-F-U][2′-F-G][2′-F-U][2′-F-A][2′-F-U] | |
| | [2′-F-C][2′-F-U][2′-F-U][2′-F-U][2′-F-G]-3′ | |
| GalNAc-Scr | N-terminal-GalNAc-Linker-5′-[2′-F-U][2′-F-U][2′-F-C][2′-F-U][2′-F-C] | 75 |
| | [2′-F-C][2′-F-G][2′-F-A][2′-F-A][2′-F-C][2′-F-G][2′-F-U][2′-F-G] | |
| | [2′-F-U][2′-F-C][2′-F-A][2′-F-C][2′-F-G][2′-F-U][2′-F-U][2′-F-U]-3′ | |
| GalNAc-HULC | N-terminal-GalNAc-Linker-5′-[2′-F-C][2′-F-A][2′-F-A][2′-F-A][2′-F-A] | 76 |
| | [2′-F-U][2′-F-G][2′-F-C][2′-F-A][2′-F-U][2′-F-A][2′-F-U][2′-F-G] | |
| | [2′-F-U][2′-F-A][2′-F-U][2′-F-C][2′-F-U][2′-F-U][2′-F-U][2′-F-G]-3′ | |
| GalNAc-HULC | N-terminal-GalNAc-Linker-5′-[2′-F-C][2′-F-A][2′-F-A][2′-F-A][2′-F-A] | 77 |
| mutant | [2′-F-U][2′-F-G][2′-F-C][2′-F-A][2′-F-U][2′-F-G][2′-F-U][2′-F-G] | |
| | [2′-F-U][2′-F-A][2′-F-U][2′-F-C][2′-F-U][2′-F-U][2′-F-U][2′-F-G]-3′ | |
| Apo-Scr | N-terminal-LRKLRKRLLLRKLRKRLL-Linker-5′-[2′-F-U][2′-F-U][2′-F-C] | 78 |
| | [2′-F-U][2′-F-C][2′-F-C][2′-F-G][2′-F-A][2′-F-A][2′-F-C][2′-F-G] | |
| | [2′-F-U][2′-F-G][2′-F-U][2′-F-C][2′-F-A][2′-F-C][2′-F-G][2′-F-U] | |
| | [2′-F-U][2′-F-U]-3′ | |
| Apo-HULC | N-terminal-LRKLRKRLLLRKLRKRLL-Linker-5′-[2′-F-C][2′-F-A][2′-F-A] | 79 |
| | [2′-F-A][2′-F-A][2′-F-U][2′-F-G][2′-F-C][2′-F-A][2′-F-U][2′-F-A] | |
| | [2′-F-U][2′-F-G][2′-F-U][2′-F-A][2′-F-U][2′-F-C][2′-F-U][2′-F-U] | |
| | [2′-F-U][2′-F-G]-3′ | |
| Apo-HULC | N-terminal-LRKLRKRLLLRKLRKRLL-Linker-5′-[2′-F-C][2′-F-A][2′-F-A] | 80 |
| mutant | [2′-F-A][2′-F-A][2′-F-U][2′-F-G][2′-F-C][2′-F-A][2′-F-U][2′-F-G] | |
| | [2′-F-U][2′-F-G][2′-F-U][2′-F-A][2′-F-U][2′-F-C][2′-F-U][2′-F-U] | |
| | [2′-F-U][2′-F-G]-3′ | |
| sgRNA | Sequence (5′→3′) | |
| HULC sgRNA | GGTCCACTACAAGTGGGAGC | 81 |
| #1 forward | | |
| HULC sgRNA | TAGGGCATAAATTATGCATC | 82 |
| #1 reverse | | |
| HULC sgRNA | GTGGCTTAAACAACACTTAC | 83 |
| #2 forward | | |
| HULC sgRNA | GATCATTGCTCTAGACTTAC | 84 |
| #2 reverse | | |
| PAH sgRNA #1 | CAAAGTATTGCGCTTATTTG | 85 |
| PAH sgRNA #2 | GCCAATCAGATTCTCAGCTA | 86 |
| RACE PCR | Sequence (5′→3′) | |
| Pair 5′ RACE | GATTACGCCAAGCTTCCATCCCACCAGGGAATCTTGGCAGCAGC | 87 |
| Pair 3′ RACE | GATTACGCCAAGCTTCCGCCTGGGAGCAGGGAGGGTTCTGCTACC | 88 |
| SHAPE | | |
| plus SHAPE | 6-FAM/TAAGAATGGACATCATTTTA | 89 |
| primer for | | |
| capillary | | |
| electrophoresis | | |
| minus SHAPE | VIC/TAAGAATGGACATCATTTTA | 90 |
| primer for | | |
| capillary | | |
| electrophoresis | | |
| ddT reaction | PET/TAAGAATGGACATCATTTTA | 91 |
| primer for | | |
| capillary | | |
| electrophoresis | | |
| ddA reaction | NED/TAAGAATGGACATCATTTTA | 92 |
| primer for | | |
| capillary | | |
| electrophoresis | | |

### References Cited in this Disclosure

1. E. Kayaalp et al., Human phenylalanine hydroxylase mutations and hyperphenylalaninemia phenotypes: a metanalysis of genotype-phenotype correlations. Am J Hum Genet 61, 1309-1317 (1997).
2. H. Murad, A. Dabboul, F. Moassas, D. Alasmar, W. Al-Achkar, Mutation spectrum of phenylketonuria in Syrian population: genotype-phenotype correlation. Gene 528, 241-247 (2013).
3. C. R. Scriver, P. J. Waters, Monogenic traits are not simple: lessons from phenylketonuria. Trends in genetics: TIG 15, 267-272 (1999).
4. Y. Anikster et al., Biallelic Mutations in DNAJC12 Cause Hyperphenylalaninemia, Dystonia, and Intellectual Disability. Am J Hum Genet 100, 257-266 (2017).
5.1. Karacic et al., Genotype-predicted tetrahydrobiopterin (BH4)-responsiveness and molecular genetics in Croatian patients with phenylalanine hydroxylase (PAH) deficiency. Mol Genet Metab 97, 165-171 (2009).
6. J. C. Guo et al., CNIT: a fast and accurate web tool for identifying protein-coding and long non-coding transcripts based on intrinsic sequence composition. Nucleic acids research 47, W516-W522 (2019).
7. J. Wang et al., Multiple functions of the RNA-binding protein HuR in cancer progression, treatment responses and prognosis. Int J Mol Sci 14, 10015-10041 (2013).
8. K. Panzitt et al., Characterization of HULC, a novel gene with striking up-regulation in hepatocellular carcinoma, as noncoding RNA. Gastroenterology 132, 330-342 (2007).
9. D. W. Collins, T. H. Jukes, Rates of transition and transversion in coding sequences since the human-rodent divergence. Genomics 20, 386-396 (1994).
10. K. E. Walters, A. M. Yu, E. J. Strobel, A. H. Settle, J. B. Lucks, Characterizing RNA structures in vitro and in vivo with selective 2′-hydroxyl acylation analyzed by primer extension sequencing (SHAPE-Seq). Methods 103, 34-48 (2016).
11. M. Kubota, C. Tran, R. C. Spitale, Progress and challenges for chemical probing of RNA structure inside living cells. Nat Chem Biol 11, 933-941 (2015).
12. B. Thony, Z. Ding, A. Martinez, Tetrahydrobiopterin protects phenylalanine hydroxylase activity in vivo: implications for tetrahydrobiopterin-responsive hyperphenylalaninemia. FEBS letters 577, 507-511 (2004).
13. H. Erlandsen, R. C. Stevens, A structural hypothesis for BH4 responsiveness in patients with mild forms of hyperphenylalaninaemia and phenylketonuria. J Inherit Metab Dis 24, 213-230 (2001).
14. E. K. Jaffe, New protein structures provide an updated understanding of phenylketonuria. Mol Genet Metab 121, 289-296 (2017).
15. S. Zhang, P. F. Fitzpatrick, Identification of the Allosteric Site for Phenylalanine in Rat Phenylalanine Hydroxylase. The Journal of biological chemistry 291, 7418-7425 (2016).
16. C. B. Frederick, K. L. Dooley, R. L. Kodell, W. G. Sheldon, F. F. Kadlubar, The effect of lifetime sodium saccharin dosing on mice initiated with the carcinogen 2-acetylaminofluorene. Fundam Appl Toxicol 12, 346-357 (1989).
17. T. Gjetting, M. Petersen, P. Guldberg, F. Guttler, Missense mutations in the N-terminal domain of human phenylalanine hydroxylase interfere with binding of regulatory phenylalanine. Am J Hum Genet 68, 1353-1360 (2001).
18. C. Carluccio, F. Fraternali, F. Salvatore, A. Fornili, A. Zagari, Structural features of the regulatory ACT domain of phenylalanine hydroxylase. PloS one 8, e79482 (2013).
19. A. Lin et al., The LINK-A lncRNA interacts with PtdIns(3,4,5)P3 to hyperactivate AKT and confer resistance to AKT inhibitors. Nature cell biology 19, 238-251 (2017).
20. Y. Feng et al., Global analysis of protein structural changes in complex proteomes. Nature biotechnology 32, 1036-1044 (2014).
21. P. J. Waters, How PAH gene mutations cause hyper-phenylalaninemia and why mechanism matters: insights from in vitro expression. Hum Mutat 21, 357-369 (2003).
22. A. Gamez, B. Perez, M. Ugarte, L. R. Desviat, Expression analysis of phenylketonuria mutations. Effect on folding and stability of the phenylalanine hydroxylase protein. The Journal of biological chemistry 275, 29737-29742 (2000).
23. J. Zschocke, C. A. Graham, D. J. Carson, N. C. Nevin, Phenylketonuria mutation analysis in Northern Ireland: a rapid stepwise approach. Am J Hum Genet 57, 1311-1317 (1995).
24. C. A. O'Neill et al., Molecular analysis of PKU in Ireland. Acta Paediatr Suppl 407, 43-44 (1994).
25. Y. Huang, Preclinical and Clinical Advances of GalNAc-Decorated Nucleic Acid Therapeutics. Mol Ther Nucleic Acids 6, 116-132 (2017).
26. M. Sanford, G. M. Keating, Sapropterin: a review of its use in the treatment of primary hyperphenylalaninaemia. Drugs 69, 461-476 (2009).
27. L. L. Santos et al., Variations in genotype-phenotype correlations in phenylketonuria patients. Genet Mol Res 9, 1-8 (2010).
28. D. Bercovich et al., Genotype-phenotype correlations analysis of mutations in the phenylalanine hydroxylase (PAH) gene. J Hum Genet 53, 407-418 (2008).
29. S. F. Garbade et al., Allelic phenotype values: a model for genotype-based phenotype prediction in phenylketonuria. Genet Med 21, 580-590 (2019).
30. N. Blau, A. Martinez, G. F. Hoffmann, B. Thony, DNAJC12 deficiency: Anew strategy in the diagnosis of hyperphenylalaninemias. Mol Genet Metab 123, 1-5 (2018).
31. C. O. Harding et al., Complete correction of hyperphenylalaninemia following liver-directed, recombinant AAV2/8 vector-mediated gene therapy in murine phenylketonuria. Gene Ther 13, 457-462 (2006).
32. M. J. Smola et al., SHAPE reveals transcript-wide interactions, complex structural domains, and protein interactions across the Xist lncRNA in living cells. Proceedings of the National Academy of Sciences of the United States of America 113, 10322-10327 (2016).
33. C. K. Kwok, Y. Ding, Y. Tang, S. M. Assmann, P. C. Bevilacqua, Determination of in vivo RNA structure in low-abundance transcripts. Nat Commun 4, 2971 (2013).
34. H. H. Qi et al., Histone H4K20/H3K9 demethylase PHF8 regulates zebrafish brain and craniofacial development. Nature 466, 503-507 (2010).
35. N. Fusaki, H. Ban, A. Nishiyama, K. Saeki, M. Hasegawa, Efficient induction of transgene-free human pluripotent stem cells using a vector based on Sendai virus, an RNA virus that does not integrate into the host genome. Proc Jpn Acad Ser B Phys Biol Sci 85, 348-362 (2009).
36. N. E. Sanjana, O. Shalem, F. Zhang, Improved vectors and genome-wide libraries for CRISPR screening. Nature methods 11, 783-784 (2014).
37. A. Lin et al., The LINK-A lncRNA activates normoxic HIFlalpha signalling in triple-negative breast cancer. Nature cell biology 18, 213-224 (2016).
38. J. Ule, K. Jensen, A. Mele, R. B. Darnell, CLIP: a method for identifying protein-RNA interaction sites in living cells. Methods 37, 376-386 (2005).
39. L. Yang et al., ncRNA- and Pc2 methylation-dependent gene relocation between nuclear structures mediates gene activation programs. Cell 147, 773-788 (2011).
40. A. Fey et al., Establishment of a real-time PCR-based approach for accurate quantification of bacterial RNA targets in water, using Salmonella as a model organism. Appl Environ Microbiol 70, 3618-3623 (2004).
41. Z. Xing et al., lncRNA Directs Cooperative Epigenetic Regulation Downstream of Chemokine Signals. Cell 159, 1110-1125 (2014).
42. L. Yang et al., lncRNA-dependent mechanisms of androgen-receptor-regulated gene activation programs. Nature 500, 598-602 (2013).
43. R. Shiman, T. Xia, M. A. Hill, D. W. Gray, Regulation of rat liver phenylalanine hydroxylase. II. Substrate binding and the role of activation in the control of enzymatic activity. The Journal of biological chemistry 269, 24647-24656 (1994).
44. S. W. Gersting et al., Activation of phenylalanine hydroxylase induces positive cooperativity toward the natural cofactor. The Journal of biological chemistry 285, 30686-30697 (2010).
45. S. Milstien, S. Kaufman, Studies on the phenylalanine hydroxylase system in liver slices. The Journal of biological chemistry 250, 4777-4781 (1975).
46. A. Sali, T. L. Blundell, Comparative protein modelling by satisfaction of spatial restraints. J Mol Biol 234, 779-815 (1993).
47. M. Parisien, F. Major, The MC-Fold and MC-Sym pipeline infers RNA structure from sequence data. Nature 452, 51-55 (2008).
48. Y. Huang, S. Liu, D. Guo, L. Li, Y. Xiao, A novel protocol for three-dimensional structure prediction of RNA-protein complexes. Sci Rep 3, 1887 (2013).
49. Y. Yan, D. Zhang, P. Zhou, B. Li, S. Y. Huang, HDOCK: a web server for protein-protein and protein-DNA/RNA docking based on a hybrid strategy. Nucleic acids research 45, W365-W373 (2017).
50. I. Tuszynska, M. Magnus, K. Jonak, W. Dawson, J. M. Bujnicki, NPDock: a web server for protein-nucleic acid docking. Nucleic acids research 43, W425-430 (2015).
51. Sawin, E. A., Murali, S. G. & Ney, D. M. Differential effects of low-phenylalanine protein sources on brain neurotransmitters and behavior in C57Bl/6-Pah(enu2) mice. Mol Genet Metab 111, 452-461, doi:10.1016/j.ymgme.2014.01.015 (2014).

What is claimed is:

1. A modified nucleic acid comprising:
a) a nucleotide sequence having at least 80% identity to any one of SEQ ID NOs: 3-7; and
b) at least one nucleotide comprising a 2′-fluoro base modification.

2. The modified nucleic acid of claim 1, wherein the nucleotide sequence has at least 80% identity to any one of SEQ ID NOs: 5-7.

3. The modified nucleic acid of claim 1, wherein the nucleotide sequence has at least 80% identity to SEQ ID NO:6.

4. The modified nucleic acid of claim 1, wherein the modified nucleic acid has a length of 15 to 40 nucleotides.

5. The modified nucleic acid of claim 1, wherein at least one of the nucleotides of the modified nucleic acid is a ribonucleotide.

6. The modified nucleic acid of claim 5, wherein a majority of the nucleotides of the modified nucleic acid are ribonucleotides.

7. The modified nucleic acid of claim 1, wherein at least 25% of the nucleotides comprise a 2′-fluoro base modification.

8. The modified nucleic acid of claim 7, wherein all of the internal nucleotides comprise a 2′-fluoro base modification.

9. The modified nucleic acid of claim 1, wherein the modified nucleic acid comprises at least one phosphorothioate bond.

10. The modified nucleic acid of claim 1, wherein the 5′ terminal nucleotide and/or the 3′ terminal nucleotide is attached via phosphorothioate bond.

11. The modified nucleic acid of claim 1, wherein the modified nucleic acid comprises a tag attached to the 3′ terminal nucleotide and/or the 5′ terminal nucleotide.

12. The modified nucleic acid of claim 11, wherein the tag comprises an apolipoprotein E peptide.

13. The modified nucleic acid of claim 12, wherein the apolipoprotein E peptide comprises an amino acid sequence having at least 90% identity to SEQ ID NO:9.

14. The modified nucleic acid of claim 11, wherein the tag comprises N-acetylgalactosamine.

15. The modified nucleic acid of claim 1, wherein the modified nucleic acid increases the affinity of a phenylalanine hydroxylase (PAH) for a PAH substrate and/or a PAH cofactor.

16. A nanoparticle comprising the modified nucleic acid of claim 1.

17. A pharmaceutical preparation comprising:
(a) the modified nucleic acid of any one of claims 1 to 15 or the nanoparticle of claim 16; and
(b) a pharmaceutically acceptable carrier.

18. A method for treating a subject diagnosed with or suspected to have phenylketonuria or hyperphenylalaninaemia, the method comprising administering to the subject a therapeutically effective amount of the pharmaceutical preparation of claim 17.

19. The method of claim 18, wherein the subject has been diagnosed with phenylketonuria.

20. The method of claim 18, wherein the subject has been diagnosed with hyperphenylalaninaemia.

21. The method of claim 18, wherein the subject has symptoms suggestive of phenylketonuria or hyperphenylalaninaemia.

22. The method of claim 18, wherein the subject has a mutation in a phenylalanine hydroxylase gene.

23. The method of claim 22, wherein the mutation is R408W.

24. The method of claim 18, wherein the pharmaceutical preparation is administered via intravenous injection, subcutaneous injection, and/or intraperitoneal injection.

25. The method of claim 18, wherein administration of the pharmaceutical preparation results in increased enzymatic conversion of phenylalanine to tyrosine by a phenylalanine hydroxylase (PAH) in one or more cells of the subject.

26. The method of claim 18, wherein administration of the pharmaceutical preparation results in increased affinity of phenylalanine hydroxylase (PAH) for a PAH substrate and/or a PAH cofactor.

27. The method of claim 26, wherein the PAH substrate is phenylalanine.

28. The method of claim 26, wherein the PAH cofactor is tetrahydrobiopterin (BH₄).


SEQUENCE LISTING (92 sequences)

SEQ ID NO: 1 - RNA, 734 nt, Mus musculus
  source 1..734: mol_type = transcribed RNA
gtgtcatgcaagaaggaggcggcggcggcggccggagctggagggggaggaggggaggaacctgatcctcggtagcgaagggctggaggcaaacaccgagctccccctgagccgcctgggagcagggagggttctgctaccctgaggggccctccccgcggggcagaggggtttcctcagcagtgccccggagcagggaccgcgccagccagcagaaacagctagttgttgtctgcagtgaagaataattggaagcatggtgcctgtttcttttcctgtgggaggaagtcacagcatctacttctgaatctgtgcttgccttgaggaaaaacggcatggcacaggaatgtggacgctttcaaccagaggattctgtacgacggtgtcggcacacaaggagtgttccactgactgacctctttttgttttcaaatgaagaggagccaagaataggacagcttgggaaggtgagctgcgtatagctcgtcaggctgagctcatcctcgtctacggccaaacgttggtttctcatttctagaagatgaaagatgtgatgactcaggaaaaacttgctgctcgagatgtgagtcttcagatgttcctgccgccatatctgacagctgctgccaagattccctggtgggatggactcttatacctcaacaaccagaagcccaagtaaactcttctataaattgaatgactcatgcttaaaaaaaaaaacaaaaaacaaa

SEQ ID NO: 2 - RNA, 500 nt, Homo sapiens
  source 1..500: mol_type = transcribed RNA
atgggggtggaactcatgatggaattggagcctttacaagggaatgaagagacaagagctctctttatgccacgtgaggatacagcaaggccccaatctgcaagccaggaagagtcgtcacgagaaccagaccatgcaggaactctgatcgtggacatttcaacctccagaactgtgatccaaaatgcatatgtatctttggaagaaactctgaagtaaaggccggaatattctttgtttaaaacattaaaaacaaaacagaccaaagcatcaagcaagaagtttcctggcaataaactaagcacagcattattttttaaggaacacaaattaagtgttcaacctgtggcaaatttgtactttctccctgaattatgttgttatcaaagaaaaaaattgggaagcatggcaaaatatcatcaaaactgaaactagaattaaacaaaactaaattaaaatgaaataaaatgatgtccattcttaaaaaaaaaaaaaaaaaa

SEQ ID NO: 3 - RNA, 37 nt, Mus musculus
  source 1..37: mol_type = transcribed RNA
ttgggaaggtgagctgcgtatagctcgtcaggctgag

SEQ ID NO: 4 - RNA, 19 nt, Mus musculus
  source 1..19: mol_type = transcribed RNA
gagctgcgtatagctcgtc

SEQ ID NO: 5 - RNA, 34 nt, Homo sapiens
  source 1..34: mol_type = transcribed RNA
aaatgcatatgtatctttggaagaaactctgaag

SEQ ID NO: 6 - RNA, 18 nt, Homo sapiens
  source 1..18: mol_type = transcribed RNA
aaatgcatatgtatcttt

SEQ ID NO: 7 - RNA, 21 nt, Homo sapiens
  source 1..21: mol_type = transcribed RNA
caaaatgcatatgtatctttg

SEQ ID NO: 8 - RNA, 21 nt, synthetic construct
  source 1..21: mol_type = other RNA
caaaatgcatgtgtatctttg

SEQ ID NO: 9 - protein, 18 aa, synthetic construct
LRKLRKRLLLRKLRKRLL

SEQ ID NO: 10 - RNA, 16 nt, synthetic construct
  source 1..16: mol_type = other RNA
agggaggacgatgcgg

SEQ ID NO: 11 - RNA, 20 nt, synthetic construct
  source 1..20: mol_type = other RNA
gtgtcagtcacttccagcgg

SEQ ID NO: 12 - DNA, 16 bp, synthetic construct
  source 1..16: mol_type = other DNA
agggaggacgatgcgg

SEQ ID NO: 13 - DNA, 20 bp, synthetic construct
  source 1..20: mol_type = other DNA
ccgctggaagtgactgacac

SEQ ID NO: 14 - DNA, 22 bp, synthetic construct
  source 1..22: mol_type = other DNA
aggctatctggacaaatgcact

SEQ ID NO: 15 - DNA, 22 bp, synthetic construct
  source 1..22: mol_type = other DNA
tgccacaggttgaacacttaat

SEQ ID NO: 16 - DNA, 24 bp, synthetic construct
  source 1..24: mol_type = other DNA
ctcattgtagaaggtgtggtgcca

SEQ ID NO: 17 - DNA, 22 bp, synthetic construct
  source 1..22: mol_type = other DNA
  misc_feature 1: note = 5' 6-FAM
gtgtaacacgtctatacgccca

SEQ ID NO: 18 - DNA, 24 bp, synthetic construct
  source 1..24: mol_type = other DNA
  misc_feature 1: note = 5' 6-FAM
ctcattgtagaaggtgtggtgcca

SEQ ID NO: 19 - DNA, 22 bp, synthetic construct
  source 1..22: mol_type = other DNA
  misc_feature 1: note = 5' 6-FAM
agaggtcagtcagtggaacact

SEQ ID NO: 20 - DNA, 21 bp, synthetic construct
  source 1..21: mol_type = other DNA
  misc_feature 1: note = 5' 6-FAM
atagctggcagtgtagcttat

SEQ ID NO: 21 - DNA, 22 bp, synthetic construct
  source 1..22: mol_type = other DNA
  misc_feature 1: note = 5' 6-FAM
tgccacaggttgaacacttaat

SEQ ID NO: 22 - DNA, 21 bp, synthetic construct
  source 1..21: mol_type = other DNA
  misc_feature 1: note = 5' 6-FAM
agtgagctgaatagtcataat

SEQ ID NO: 23 - DNA, 22 bp, synthetic construct
  source 1..22: mol_type = other DNA
tggacgctttcaaccagaggat

SEQ ID NO: 24 - DNA, 22 bp, synthetic construct
  source 1..22: mol_type = other DNA
ctatacgcagctcaccttccca

SEQ ID NO: 25 - DNA, 22 bp, synthetic construct
  source 1..22: mol_type = other DNA
tcgtcacgagaaccagaccatg

SEQ ID NO: 26 - DNA, 23 bp, synthetic construct
  source 1..23: mol_type = other DNA
gctgtgcttagtttattgccagg

SEQ ID NO: 27 - DNA, 20 bp, synthetic construct
  source 1..20: mol_type = other DNA
ttttcccagggcgaggctta

SEQ ID NO: 28 - DNA, 23 bp, synthetic construct
  source 1..23: mol_type = other DNA
ccccactaccacaaattatgcag

SEQ ID NO: 29 - DNA, 22 bp, synthetic construct
  source 1..22: mol_type = other DNA
agagagcaaagcggtgtagtgt

SEQ ID NO: 30 - DNA, 21 bp, synthetic construct
  source 1..21: mol_type = other DNA
atcagcaaggcagagaggtgg

SEQ ID NO: 31 - DNA, 23 bp, synthetic construct
  source 1..23: mol_type = other DNA
agttacagggaagtttgggctac

SEQ ID NO: 32 - DNA, 20 bp, synthetic construct
  source 1..20: mol_type = other DNA
aggcaaaggacggctgtgag

SEQ ID NO: 33 - DNA, 22 bp, synthetic construct
  source 1..22: mol_type = other DNA
gccactgtccatgagctttcac

SEQ ID NO: 34 - DNA, 22 bp, synthetic construct
  source 1..22: mol_type = other DNA
agcatccagttccgctccatag

SEQ ID NO: 35 - DNA, 22 bp, synthetic construct
  source 1..22: mol_type = other DNA
tggatgagaggtggcaaggtac

SEQ ID NO: 36 - DNA, 22 bp, synthetic construct
  source 1..22: mol_type = other DNA
gctcaggaaggaagatgcagga

SEQ ID NO: 37 - DNA, 21 bp, synthetic construct
  source 1..21: mol_type = other DNA
caccggctgcacagattgatc

SEQ ID NO: 38 - DNA, 22 bp, synthetic construct
  source 1..22: mol_type = other DNA
gcacatccaggtccaggttctt

SEQ ID NO: 39 - DNA, 23 bp, synthetic construct
  source 1..23: mol_type = other DNA
tggtctcagtaaacttgccagga

SEQ ID NO: 40 - DNA, 22 bp, synthetic construct
  source 1..22: mol_type = other DNA
caatcactactccaacgccagc

SEQ ID NO: 41 - DNA, 22 bp, synthetic construct
  source 1..22: mol_type = other DNA
gcaattctctgtgtggctggag

SEQ ID NO: 42 - DNA, 22 bp, synthetic construct
  source 1..22: mol_type = other DNA
gagtcccatccaaggcagcttt

SEQ ID NO: 43 - DNA, 22 bp, synthetic construct
  source 1..22: mol_type = other DNA
ggtggaatgaagtagaaggccg

SEQ ID NO: 44 - DNA, 22 bp, synthetic construct
  source 1..22: mol_type = other DNA
tgggtgctcaaggtgatatgga

SEQ ID NO: 45 - DNA, 22 bp, synthetic construct
  source 1..22: mol_type = other DNA
ctcatcaacaacgcagccactc

SEQ ID NO: 46 - DNA, 22 bp, synthetic construct
  source 1..22: mol_type = other DNA
ccggaagtcaaacagagcatgg

SEQ ID NO: 47 - DNA, 21 bp, synthetic construct
  source 1..21: mol_type = other DNA
aggtcggtgtgaacggatttg

SEQ ID NO: 48 - DNA, 23 bp, synthetic construct
  source 1..23: mol_type = other DNA
tgtagaccatgtagttgaggtca

SEQ ID NO: 49 - DNA, 21 bp, synthetic construct
  source 1..21: mol_type = other DNA
gccagaggataaagcaagctg

SEQ ID NO: 50 - DNA, 23 bp, synthetic construct
  source 1..23: mol_type = other DNA
tcaccttccaaattacctccttt

SEQ ID NO: 51 - DNA, 21 bp, synthetic construct
  source 1..21: mol_type = other DNA
ttcactatctggtgctgaagc

SEQ ID NO: 52 - DNA, 22 bp, synthetic construct
  source 1..22: mol_type = other DNA
taccgaaggaggcagattacgc

SEQ ID NO: 53 - DNA, 22 bp, synthetic construct
  source 1..22: mol_type = other DNA
gtcgagccaatcacaggagaag

SEQ ID NO: 54 - DNA, 22 bp, synthetic construct
  source 1..22: mol_type = other DNA
gtgtgaaggagaaccagtggac

SEQ ID NO: 55 - DNA, 23 bp, synthetic construct
  source 1..23: mol_type = other DNA
cacagaggaatccaagtaacggc

SEQ ID NO: 56 - DNA, 22 bp, synthetic construct
  source 1..22: mol_type = other DNA
ataaccaggcggtggcagtgaa

SEQ ID NO: 57 - DNA, 22 bp, synthetic construct
  source 1..22: mol_type = other DNA
ccagcttcatgtcactgactcc

SEQ ID NO: 58 - DNA, 22 bp, synthetic construct
  source 1..22: mol_type = other DNA
agaaaggagggtgatgctgtgt

SEQ ID NO: 59 - DNA, 22 bp, synthetic construct
  source 1..22: mol_type = other DNA
ccctgtgaaggttgaaatggct

SEQ ID NO: 60 - DNA, 22 bp, synthetic construct
  source 1..22: mol_type = other DNA
cttttcctgtgggaggaagtca

SEQ ID NO: 61 - DNA, 22 bp, synthetic construct
  source 1..22: mol_type = other DNA
aggatgagcttcccaagctgtc

SEQ ID NO: 62 - DNA, 21 bp, synthetic construct
  source 1..21: mol_type = other DNA
cagagggtgggaggaagtcac

SEQ ID NO: 63 - DNA, 22 bp, synthetic construct
  source 1..22: mol_type = other DNA
atcctctggttgaaagcgtcca

SEQ ID NO: 64 - DNA, 22 bp, synthetic construct
  source 1..22: mol_type = other DNA
ggcacacactcatcctcgtcta

SEQ ID NO: 65 - DNA, 22 bp, synthetic construct
  source 1..22: mol_type = other DNA
cttgggcttctggttgttgagg

SEQ ID NO: 66 - DNA, 23 bp, synthetic construct
  source 1..23: mol_type = other DNA
gcctgtttcttttcctaggagcc

SEQ ID NO: 67 - DNA, 22 bp, synthetic construct
  source 1..22: mol_type = other DNA
ctcaatgcccaccacagttact

SEQ ID NO: 68 - RNA, 23 nt, synthetic construct
  source 1..23: mol_type = other RNA
ccaaaatgcatatgtatctttgg

SEQ ID NO: 69 - RNA, 23 nt, synthetic construct
  source 1..23: mol_type = other RNA
ccaaaatgcatgtgtatctttgg

SEQ ID NO: 70 - RNA, 22 nt, synthetic construct
  source 1..22: mol_type = other RNA
ggtgagctgcgtatagctcgtc

SEQ ID NO: 71 - RNA, 22 nt, synthetic construct
  source 1..22: mol_type = other RNA
ggtgagctgcgtgtagctcgtc

SEQ ID NO: 72 - RNA, 21 nt, synthetic construct
  source 1..21: mol_type = other RNA
  modified_base 1..21: mod_base = OTHER; note = 2'-fluoro modification
ttctccgaacgtgtcacgttt

SEQ ID NO: 73 - RNA, 21 nt, synthetic construct
  source 1..21: mol_type = other RNA
  modified_base 1..21: mod_base = OTHER; note = 2'-fluoro modification
caaaatgcatatgtatctttg

SEQ ID NO: 74 - RNA, 21 nt, synthetic construct
  source 1..21: mol_type = other RNA
  modified_base 1..21: mod_base = OTHER; note = 2'-fluoro modification
caaaatgcatgtgtatctttg

SEQ ID NO: 75 - RNA, 21 nt, synthetic construct
  source 1..21: mol_type = other RNA
  modified_base 1..21: mod_base = OTHER; note = 2'-fluoro modification
  misc_feature 1: note = A GalNAc tag is attached through a linker
ttctccgaacgtgtcacgttt

SEQ ID NO: 76 - RNA, 21 nt, synthetic construct
  source 1..21: mol_type = other RNA
  modified_base 1..21: mod_base = OTHER; note = 2'-fluoro modification
  misc_feature 1: note = A GalNAc tag is attached through a linker
caaaatgcatatgtatctttg

SEQ ID NO: 77 - RNA, 21 nt, synthetic construct
  source 1..21: mol_type = other RNA
  modified_base 1..21: mod_base = OTHER; note = 2'-fluoro modification
  misc_feature 1: note = A GalNAc tag is attached through a linker
caaaatgcatgtgtatctttg

SEQ ID NO: 78 - RNA, 21 nt, synthetic construct
  source 1..21: mol_type = other RNA
  modified_base 1..21: mod_base = OTHER; note = 2'-fluoro modification
  misc_feature 1: note = An ApoE tag is attached through a linker
ttctccgaacgtgtcacgttt

SEQ ID NO: 79 - RNA, 21 nt, synthetic construct
  source 1..21: mol_type = other RNA
  modified_base 1..21: mod_base = OTHER; note = 2'-fluoro modification
  misc_feature 1: note = An ApoE tag is attached through a linker
caaaatgcatatgtatctttg

SEQ ID NO: 80 - RNA, 21 nt, synthetic construct
  source 1..21: mol_type = other RNA
  modified_base 1..21: mod_base = OTHER; note = 2'-fluoro modification
  misc_feature 1: note = An ApoE tag is attached through a linker
caaaatgcatgtgtatctttg

SEQ ID NO: 81 - DNA, 20 bp, synthetic construct
  source 1..20: mol_type = other DNA
ggtccactacaagtgggagc

SEQ ID NO: 82 - DNA, 20 bp, synthetic construct
  source 1..20: mol_type = other DNA
tagggcataaattatgcatc

SEQ ID NO: 83 - DNA, 20 bp, synthetic construct
  source 1..20: mol_type = other DNA
gtggcttaaacaacacttac

SEQ ID NO: 84 - DNA, 20 bp, synthetic construct
  source 1..20: mol_type = other DNA
gatcattgctctagacttac

SEQ ID NO: 85 - DNA, 20 bp, synthetic construct
  source 1..20: mol_type = other DNA
caaagtattgcgcttatttg

SEQ ID NO: 86 - DNA, 20 bp, synthetic construct
  source 1..20: mol_type = other DNA
gccaatcagattctcagcta

SEQ ID NO: 87 - DNA, 44 bp, synthetic construct
  source 1..44: mol_type = other DNA
gattacgccaagcttccatcccaccagggaatcttggcagcagc

SEQ ID NO: 88 - DNA, 45 bp, synthetic construct
  source 1..45: mol_type = other DNA
gattacgccaagcttccgcctgggagcagggagggttctgctacc

SEQ ID NO: 89 - DNA, 20 bp, synthetic construct
  source 1..20: mol_type = other DNA
  misc_feature 1: note = 6-FAM label
taagaatggacatcatttta

SEQ ID NO: 90 - DNA, 20 bp, synthetic construct
  source 1..20: mol_type = other DNA
  misc_feature 1: note = VIC label
taagaatggacatcatttta

SEQ ID NO: 91 - DNA, 20 bp, synthetic construct
  source 1..20: mol_type = other DNA
  misc_feature 1: note = PET label
taagaatggacatcatttta

SEQ ID NO: 92 - DNA, 20 bp, synthetic construct
  source 1..20: mol_type = other DNA
  misc_feature 1: note = NED label
taagaatggacatcatttta